(19) United States
(12) Patent Application Publication
(10) Pub. No.: US 2024/0238429 A1
(43) Pub. Date: Jul. 18, 2024

(54) DELIVERY CONSTRUCTS FOR TRANSCYTOSIS AND RELATED METHODS

(71) Applicant: Thornhill Therapeutics, Inc, San Francisco, CA (US)

(72) Inventors: Randall J. Mrsny, Los Altos Hills, CA (US); Tahir Mahmood, Burlingame, CA (US); Charles Olson, South San Francisco, CA (US); Weijun Feng, Danville, CA (US)

(21) Appl. No.: 18/241,744

(22) Filed: Sep. 1, 2023

Related U.S. Application Data
(63) Continuation of application No. 17/235,567, filed on Apr. 20, 2021, now abandoned.
Application No. 17/235,567 is a continuation of application No. 16/902,256, filed on Jun. 15, 2020, now Pat. No. 11,027,020.
Application No. 16/902,256 is a continuation of application No. PCT/US2019/060356, filed on Nov. 7, 2019.
(60) Provisional application No. 62/756,889, filed on Nov. 7, 2018.
(60) Provisional application No. 62/888,133, filed on Aug. 16, 2019.
(60) Provisional application No. 62/888,238, filed on Aug. 16, 2019.

(51) Int. Cl. A61K 47/64; A61K 38/20; A61P 1/00; C07K 1/18

(52) U.S. Cl. CPC A61K 47/642; A61K 38/20; A61P 1/00; C07K 1/18

(57) ABSTRACT

The present disclosure provides non-naturally occurring fusion molecules comprising therapeutic cargo moieties, such as IL-22 with a carrier. The disclosure also provides methods and compositions for the production, purification, refolding, formulation, and administration of fusion molecules. Methods and for using the purified molecules to treat and prevent diseases or disorders are also provided herein.

### CROSS-REFERENCE

This application is a continuation application of U.S. patent application Ser. No. 17/235,567, filed Apr. 20, 2021, which is a continuation application of U.S. patent application Ser. No. 16/902,256, filed Jun. 15, 2020, which is a continuation application of International Application No. PCT/US2019/060356, filed Nov. 7, 2019, which claims the benefit of U.S. Provisional Application No. 62/756,889, filed Nov. 7, 2018, U.S. Provisional Application No. 62/888,133, filed Aug. 16, 2019, and U.S. Provisional Application No. 62/888,238 filed Aug. 16, 2019, which applications are incorporated herein by reference in their entireties.

### SEQUENCE LISTING

The instant application contains a Sequence Listing which has been submitted electronically in XML format and is hereby incorporated by reference in its entirety. Said XML copy, created on Mar. 26, 2024, is named 67482-716_304_SL.xml and is 138,831 bytes in size.

### BACKGROUND

The gut epithelium has thwarted efforts to orally administer large therapeutic molecules such as proteins because proteins cannot diffuse across the intact epithelial barrier or cross the barrier through the tight junctions. Once taken up by an epithelial cell, a therapeutic protein can enter the destructive lysosomal trafficking pathway, or can be released back into the intestinal lumen. This inability to be readily transported across the intestinal epithelium can be a limiting factor in developing commercially viable oral formulations, particularly for polypeptide-based therapeutics.

Parenteral administration such as intravenous or subcutaneous administration can be a solution, but these administration routes can often create considerable side effects, lower the therapeutic efficacy, and reduce patient convenience that can negatively affect compliance. There is a need for improved compositions and methods for transporting therapeutics across an epithelium, e.g., a gut epithelium.

Additionally, purification and refolding of biologically active polypeptides in order to obtain correctly folded, biologically active, and stable polypeptides in high yields and with low endotoxin levels is still considered one of the most challenging aspects for a cost- and resource-effective production of biological therapeutics (e.g., polypeptides). Thus, there is also a need for improved methods for the production (fermentation, refolding, purification, and formulation) of such biologically active molecules.

### SUMMARY

In the various aspects, the present disclosure provides a delivery construct comprising an amino acid sequence set forth in SEQ ID NO: 15 or SEQ ID NO: 17, or an amino acid sequence having at least 90%, 95% or 99% sequence identity thereto.

In the various aspects, the present disclosure provides a delivery construct comprising a carrier consisting of an amino acid sequence set forth in SEQ ID NO: 7 or SEQ ID NO: 9. In some instances, the carrier is coupled to a heterologous payload. In some instances, the heterologous payload is a human IL-22. In some instances, the human IL-22 consists of an amino acid sequence set forth in SEQ ID NO: 11. In some instances, the carrier is coupled covalently or non-covalently to the IL-22. In some instances, the carrier is coupled covalently to the IL-22 via a spacer. In some instances, the spacer consists of an amino acid sequence set forth in SEQ ID NO: 13. In some instances, the delivery construct consists of an amino acid sequence set forth in SEQ ID NO: 15 or SEQ ID NO: 17.

Provided herein is a method of treating an inflammatory disease in a subject, the method comprising administering to the subject an effective amount of a delivery construct described herein (e.g., SEQ ID NO: 15 or SEQ ID NO: 17). In some instances, the inflammatory disease is hepatitis, obesity, fatty liver disease, liver inflammation, or pancreatitis, Crohn's disease, ulcerative colitis, pouchitis, proctitis, multiple sclerosis, systemic lupus erythematosus, graft versus host disease, rheumatoid arthritis, or psoriasis. In some instances, the disease is Crohn's disease or ulcerative colitis.

Described herein, in certain embodiments, are methods for obtaining a purified non-naturally occurring fusion protein, the method comprising: performing anion exchange chromatography on a mixture comprising the non-naturally occurring fusion protein to obtain a first fraction comprising the non-naturally occurring fusion protein; wherein the non-naturally occurring fusion protein comprises IL-22 and a carrier. In some embodiments, wherein performing anion exchange chromatography comprises binding the non-naturally occurring fusion protein to anionic exchange resin and providing an increasing salt gradient for subsequent elution of the non-naturally occurring fusion protein to obtain the first fraction. In some embodiments, performing anion exchange chromatography comprises contacting the mixture with a resin comprising amine-functionalized polymethacrylate beads. In some embodiments, the resin is an NH₂-750F resin.

In some embodiments, the method further comprises refolding the non-naturally occurring fusion protein prior to performing anion exchange chromatography. In some embodiments, refolding the non-naturally occurring fusion protein comprises contacting chaotrope-solubilized protein from inclusion bodies with a refolding solution, wherein the refolding solution comprises: arginine (0.75 M to 1.25 M); glycerol (2% to 20% v/v); cysteine (0.5 mM to 10 mm); and cystamine (0.2 mM to 10 mM); wherein the refolding solution has a pH of between 7.5 and 8.5. In some embodiments, the arginine is present in the refolding solution at a concentration of between 0.9 M and 1.1 M; glycerol is present in the refolding solution at a concentration of between 7% and 13% (w/w); cysteine is present in the refolding solution at a concentration of between 1.5 mM and 6 mM; cystamine is present in the refolding solution at a concentration of between 0.6 mM and 3 mM; and the refolding solution has a pH of between 7.8 and 8.2.

In some embodiments, the method further comprises subjecting a sample comprising the first fraction to a hydroxyapatite resin to obtain a second fraction comprising the non-naturally occurring fusion protein. In some embodiments, the hydroxyapatite resin is a CaPure-hydroxyapatite resin. In some embodiments, the method further comprises performing cation exchange chromatography on a sample comprising the first fraction. In some embodiments, performing cation exchange chromatography comprises contacting the sample comprising the first fraction with a resin comprising sulfate-functionalized polymethacrylate beads. In some embodiments, the resin is a TOYOPEARL Sulfate-650F resin.

In some embodiments, upon contact with a cell, the carrier promotes endocytosis or transcytosis of the non-naturally occurring fusion protein. In some embodiments, upon contact with the cell, the carrier promotes transcytosis of the non-naturally occurring fusion protein. In some embodiments, the cell is a gut epithelial cell. In some embodiments, the gut epithelial cell is a polarized gut epithelial cell. In some embodiments, the carrier is a truncated variant of a naturally occurring or non-naturally occurring cholix polypeptide. In some embodiments, the carrier has at least 70%, 80%, 85%, 90%, 95%, 98%, 99%, or 100% sequence identity to any one of SEQ ID NOS: 1, 2, 3, 4, 5, 6, 7, 8, 9, 22, or 23. In some embodiments, the non-naturally occurring fusion protein has at least 70%, 80%, 85%, 90%, 95%, 98%, 99%, or 100% sequence identity to the sequence of any one of SEQ ID NOS: 14-21. In some embodiments, IL-22 has at least 85%, 90%, 95%, 98%, 99% or 100% sequence identity to any one of SEQ ID NOS: 10, 11, or 12.

In some embodiments, the carrier, by itself, has a first isoelectric point (pI) and the IL-22, by itself, has a second isoelectric point, wherein the first isoelectric point is at least 1 pH unit, at least 1.5 pH units, at least 1.7 pH units, or at least 2 pH units lower than the second isoelectric point. For instance, in some embodiments, the carrier has a pI of between 4.8 and 5.4, between 4.9 and 5.3, between 5.0 and 5.2, such as a pI of about 5.1. In some embodiments, the pI of the IL-22 is between about 6.8 and 7.4, such as between about 6.9 and 7.3, between about 7.0 ant 7.2, such as about 7.1. In some embodiments, the non-naturally occurring fusion protein is obtained by any of the methods described herein.

Described herein, in certain embodiments, are methods of refolding a non-naturally occurring fusion protein comprising a carrier and IL-22, the method comprising: (i) contacting inclusion bodies comprising the non-naturally occurring fusion protein with a solubilization solution comprising a chaotropic agent to produce a soluble non-naturally occurring fusion protein; (iii) contacting the non-naturally occurring fusion protein with a refolding solution, wherein the refolding solution comprises: arginine (0.75 M to 1.25 M); glycerol (2% to 20% v/v); cysteine (0.5 mM to 10 mm); and cystamine (0.2 mM to 10 mM); wherein the refolding solution has a pH of between 7.5 and 8.5.

In some embodiments, arginine is present in the refolding solution at a concentration of between 0.9 M and 1.1 M; glycerol is present in the refolding solution at a concentration of between 7% and 13% (w/w); cysteine is present in the refolding solution at a concentration of between 1.5 mM and 6 mM; cystamine is present in the refolding solution at a concentration of between 0.6 mM and 3 mM; and the refolding solution has a pH of between 7.8 and 8.2.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 schematically shows a setup comprising an apical chamber above an epithelial cell monolayer and a basal chamber below such epithelial cell monolayer. For apical to basolateral permeability, test articles (e.g., delivery constructs, payloads, etc.) were applied to the apical (A) side and the amount of permeated (e.g., transcytosed) material was determined on the basolateral (B) side.

FIG. 2 shows that a delivery construct (SEQ ID NO: 15), which includes a carrier (SEQ ID NO: 7) coupled to an IL-22 (SEQ ID NO: 11) via a spacer (SEQ ID NO: 13), transported the IL-22 payload across intact, polarized, Caco-2 gut epithelial cell monolayers in a time-dependent manner (the amount of protein on the basolateral site was measured 15, 30, and 45 minutes after the delivery construct was applied to the basal membrane of the monolayer as described above in FIG. 1). The data further shows that when the delivery construct with a carrier of SEQ ID NO: 7 and IL-22 (SEQ ID NO: 11) is applied to the to the Caco-2 epithelial cells, about 2-3 fold more IL-22 crossed the Caco-2 epithelial cell monolayer as compared to when an IL-22 (SEQ ID NO: 12) was not coupled to a carrier.

FIG. 3 shows that a delivery construct (SEQ ID NO: 15) resulted in IL-22 (SEQ ID NO: 11) being transported across intact and polarized SMI-100 gut epithelial cell monolayers in a time-dependent manner (the amount of protein in the basolateral chamber was measured at 15, 30, and 45 minutes after the delivery construct was applied to the basal membrane of the monolayer). The data further shows that when the delivery construct including the carrier with SEQ ID NO: 7 coupled to IL-22 (SEQ ID NO: 11) is applied to the SMI-100 epithelial cells, about 2-3 fold more IL-22 crossed the SMI-100 epithelial cell monolayer as compared to when an IL-22 (SEQ ID NO: 12) was not coupled to a carrier.

FIG. 4 demonstrates that a delivery construct (SEQ ID NO: 15) including a carrier (SEQ ID NO: 7) coupled to an IL-22 (SEQ ID NO: 11) via a spacer (SEQ ID NO: 13) results in IL-22 being transported in significant amounts across an intact and polarized gut epithelium in vivo. The apical site of the gut epithelium is highlighted by white arrow #1. The lamina propria is abbreviated as “l.p.” The outer basal membrane of the polarized epithelium is highlighted by white arrow #2. IL-22 localization is indicated white arrow #3).

FIG. 5A demonstrates STAT3 phosphorylation in murine FL83 B cells as a function of agonist concentration (in pM), wherein the agonist is recombinant human IL-22 (rhIL-22, SEQ ID NO: 12) or recombinant murine IL-22 (rmIL-22; MAVLQKSMSFSLMGTLAASCLLLIALWAQE ANALPVNTRCKLEVSNFQQPYIVNRTFMLAKEASLADNNTDVRLIGEKLFRGVSAKDQCYL MKQVLNFTLEDVLLPQSDRFQPYMQEVVPFLTKLSNQLSSCHISGDDQNIQKNVRRLKETV KKLGESGEIKAIGELDLLFMSLRNACV (SEQ ID NO: 30)). The data show that rhIL-22 and rmIL-22 induced STAT3 phosphorylation in a concentration-dependent manner, demonstrating that the human IL-22 protein can bind and induce signal transduction through mouse IL-22 receptors as effectively as mouse IL-22.

FIG. 5B demonstrates that rhIL-22 (SEQ ID NO: 12) and rmIL-22 also induced STAT3 phosphorylation in murine Hepa1-6 cells.

FIG. 6A demonstrates that rhIL-22 (SEQ ID NO: 12) and rmIL-22 induced STAT5 phosphorylation in murine FL83 B cells in a dose-dependent manner, demonstrating that the human IL-22 protein can bind and induce signal transduction through mouse IL-22 receptors as effectively as mouse IL-22.

FIG. 6B demonstrates that rhIL-22 (SEQ ID NO: 12) and rmIL-22 also induced STAT5 phosphorylation in murine Hepa1-6 cells.

FIG. 7A shows an overview of an in vivo study design and timeline for comparing peroral (p.o.) administration of the delivery construct with SEQ ID NO: 15 at two once daily dose levels, 1 mg/kg and 30 mg/kg, and one once daily administration (intraperitoneal (i.p.)) of rhIL-22 at 4 mg/kg. The in vivo study was performed in Normal chow-fed, 8-10 week old, ˜23 g, female C57BL/6 mice. Colitis was chemically induced with 2.5% dextran sulfate sodium (DSS) in drinking water, ad libitum. Study endpoints included percent change in body weight, disease activity index, colon length and weight, and histopathology.

FIG. 7B shows characteristics of the experimental and control groups used in the study described in FIG. 7A.

FIG. 8A shows the percent (%) change in body weight of animals at study day 10 relative to day 0 of the various experimental and control groups. Baseline (day 0 vs. 10) body weight change by rhIL-22 with SEQ ID NO: 12 or the delivery construct with SEQ ID NO: 15 relative to vehicle was not significant as assessed by 1-way ANOVA. Positive model control (cyclosporine) CsA was significantly different relative to Vehicle as assessed by a 1-way ANOVA.

FIG. 8B shows the change in body weight of experimental and control animals over the first 10 study days. CsA group mean body weight over the study period was significantly improved relative to vehicle control day 6 through day 10 as assessed by 2-way ANOVA(*, ** , ***) rhIL-22 (i.p.) body weight was significantly improved relative to vehicle control at day 10 as assessed by 2-way ANOVA (*), *p<0.05, **p<0.01, ***p<0.001.

FIG. 9 shows results of plasma ELISA experiments to determine IL-22 plasma levels. The results show that plasma rhIL-22 concentrations trended towards increased levels in plasma after oral gavage of both 1 and 30 mg/kg doses of the delivery construct with SEQ ID NO: 15 in a dose-dependent manner (*p<0.05, **p<0.01, ***p<0.001. ****p<0.0001; Mean+SEM; n=2 Naïve, 5 Vehicle, 5 CsA, 10 others)

FIG. 10A shows an overview of a single dose in vivo study designed to identify biomarker(s) of target engagement after single (acute dosing) of rhIL-22 in healthy CD-1 mice.

FIG. 10B shows an overview of a multiple dose (sub-chronic dosing) in vivo study designed to identify biomarker(s) of target engagement after single and multiple doses of rhIL-22 in healthy CD-1 mice.

FIG. 11A shows that acute and sub-chronic administration of rhIL-22 increases IL-22 concentration consistently.

FIG. 11B shows some pharmacokinetic parameters measured during the acute and sub-chronic dosing experiments described in FIG. 11A.

FIG. 12A shows the induced circulating murine C-reactive protein (mCRP) concentration in response to 1 day after acute dosing of rhIL-22 (SEQ ID NO: 12) and after 5 days of sub-chronic dosing of rhIL-22 (SEQ ID NO: 12). The results show a time-dependent increase in circulating mCRP in both study groups.

FIG. 12B shows fold change of plasma mCRP concentration 1 day after acute dosing of rhIL-22 (SEQ ID NO: 12).

FIG. 12C shows fold change of plasma mCRP concentration after 5 days of sub-chronic dosing of rhIL-22 (SEQ ID NO: 12).

FIG. 13A shows the induced circulating murine serum amyloid protein A (mSAA) concentration in response to 1 day after acute dosing of rhIL-22 (SEQ ID NO: 12) and after 5 days of sub-chronic dosing of rhIL-22 (SEQ ID NO: 12). The results show a time-dependent increase in circulating mSAA in both study groups, with approximate plasma peak concentrations about 8 hours after administration

FIG. 13B shows fold change of plasma mSAA concentration 1 day after acute dosing of rhIL-22 (SEQ ID NO: 12).

FIG. 13C shows fold change of plasma mSAA concentration after 5 days of sub-chronic dosing of rhIL-22 (SEQ ID NO: 12).

FIG. 14A shows the induced circulating regenerating islet-derived protein 3β (Reg3β) in response to 1 day after acute dosing of rhIL-22 (SEQ ID NO: 12) and after 5 days of sub-chronic dosing rhIL-22 (SEQ ID NO: 12).

FIG. 14B shows fold change of plasma Reg3β concentration 1 day after acute dosing of rhIL-22 (SEQ ID NO: 12).

FIG. 14C shows fold change of plasma Reg3β concentration after 5 days of sub-chronic dosing of rhIL-22 (SEQ ID NO: 12).

FIG. 15 illustrates a flow chart showing how improved manufacturability may enable clinical translation.

FIG. 16 illustrates that SEQ ID NO: 15 is a heterologous fusion protein comprising a carrier domain (SEQ ID NO: 7) linked via a spacer (SEQ ID NO: 13) to interleukin-22 (IL-22, SEQ ID NO: 11). The heterologous fusion proteins may be sequestered in E. coli inclusion bodies (IB) and thus require refolding and/or purification.

FIG. 17 illustrates a flow chart summarizing the refolding, purification, and formulation of heterologous fusion proteins described herein, such as, for example SEQ ID NOS: 14-21, and fusion proteins comprising a carrier of SEQ ID NO: 1-9, 22, or 23.

FIG. 18 illustrates iterative optimization for the improved refolding efficiency of the construct of SEQ ID NO: 15.

FIG. 19 is a size-exclusion chromatograph (SEC) showing the signal of the optimized refolded protein (SEQ ID NO: 15) and protein aggregates having higher molecular masses and thus lower retention times.

FIG. 20 illustrates an exemplary SDS-PAGE of the initial and optimized refolded protein (SEQ ID NO: 15).

FIGS. 21A-21B illustrates a size-exclusion chromatograph following a first column purification of SEQ ID NO: 15 using anion exchange resin NH₂-750F. FIG. 21A illustrates a size-exclusion chromatograph following a first column purification of SEQ ID NO: 15 using anion exchange resin NH₂-750F with a column volume of 10 mL and a bed height of 20 cm. FIG. 21B illustrates a size-exclusion chromatograph following a first column purification of SEQ ID NO: 15 using anion exchange resin NH₂-750F with column volume of 4.6 L and a bed height of 30 cm.

FIGS. 22A-22B illustrate a size exclusion chromatograph following a second column purification of SEQ ID NO: 15 using hydroxyapatite resin CaPure®. FIG. 22A illustrates a size exclusion chromatograph following a second column purification of SEQ ID NO: 15 using hydroxyapatite resin Ca⁺⁺Pure with a column volume of 5 mL, a bed height of 10 cm, and a gradient of 0-25% B, 25C. FIG. 22B illustrates a size exclusion chromatograph following a second column purification of SEQ ID NO: 15 using hydroxyapatite resin CaPure® with a column volume of 800 mL, a bed height of 21 cm, and a gradient of 0-25% B, 25CV. For each figure a corresponding SDS-PAGE of samples from the different fractions eluted from the CaPure® resin.

FIGS. 23A-23B illustrate a liquid chromatography-mass spectrophotometry (LC-MS) chromatograph following purification of heterologous fusion proteins. FIG. 23A illustrates an LC-MS chromatograph of SEQ ID NO: 17. Peak 1 illustrates correctly refolded SEQ ID NO: 17. FIG. 23B illustrates an LC-MS chromatograph of SEQ ID NO: 15. Peak 1 illustrates corrected refolded SEQ ID NO: 15. Peak 2 illustrates SEQ ID NO: 15 with the terminal methionine cleaved (illustrated by SEQ ID NO: 20). Peak 3 illustrates SEQ ID NO: 15 with the terminal methionine cleaved (illustrates by SEQ ID NO: 20) and acetylation of the resulting N-terminal amino acid.

FIG. 24 illustrates the study overview of EXAMPLE 16 for assessing transport of the delivery construct having the sequence of SEQ ID NO: 15 across the gut epithelium and into circulation after oral (abbreviated herein as P.O.) delivery of the construct.

FIGS. 25A-25B illustrate graphs showing IL-22 plasma concentration as a function of time after administration of the delivery construct of SEQ ID NO: 15. FIG. 25A illustrates total IL-22 plasma concentration as a function of time after administration of the delivery construct of SEQ ID NO: 15. FIG. 25B illustrates IL-22BP plasma concentration as a function of time after administration of the delivery construct of SEQ ID NO: 15.

FIG. 26A (vehicle) and FIG. 26B (SEQ ID NO: 15) are graphs showing IL-22 plasma concentration as a function of time for individual mice.

FIGS. 27A-27D illustrate that spacer length and coupling of payload to the N- or C-terminus of a carrier does not significantly affect a payload's biological activity. FIG. 27A illustrates the length of various amino acid spacers did not affect the induction of IL-22 receptor dimerization.

FIG. 27B illustrates that coupling the IL-22 payload to the N- or C-terminus of a carrier did not affect induction of IL-22 dimerization. FIG. 27C illustrates the length of various amino acid spacers did not affect induction of pSTAT3 activation. FIG. 27D illustrates that coupling the IL-22 payload to the N- or C-terminus of a carrier did not affect induction of pSTAT3 activation.

### DETAILED DESCRIPTION

The present disclosure describes non-naturally occurring fusion proteins (e.g. delivery constructs capable of transporting one or more heterologous payload molecules) and methods for the refolding and purification of non-naturally occurring fusion proteins. For example, the non-naturally occurring fusion protein can be an IL-22 delivery construct described herein.

The below terms are discussed to illustrate meanings of the terms as used in this specification, in addition to the understanding of these terms by those of skill in the art. As used herein and in the appended claims, the singular forms “a,” “an,” and, “the” include plural referents unless the context clearly dictates otherwise. It is further noted that the claims can be drafted to exclude any optional element. As such, this statement is intended to serve as antecedent basis for use of such exclusive terminology as “solely,” “only,” and the like in connection with the recitation of claim elements, or use of a “negative” limitation.

Certain ranges or numbers are presented herein with numerical values being preceded by the term “about.” The term “about” is used herein shall mean plus or minus 1%, 2%, 3%, 4%, or 5% of the number that the term refers to. As used herein, the terms “subject” and “individual,” are used interchangeably and can be any animal, including mammals (e.g., a human or non-human animal).

As used herein, the terms “treat,” “treating,” or “treatment,” and other grammatical equivalents, include alleviating, abating or ameliorating one or more symptoms of a disease or condition, ameliorating, preventing or reducing the appearance, severity or frequency of one or more additional symptoms of a disease or condition, ameliorating or preventing the underlying causes of one or more symptoms of a disease or condition, inhibiting the disease or condition, such as, for example, arresting the development of the disease or condition, relieving the disease or condition, causing regression of the disease or condition, relieving a condition caused by the disease or condition, or inhibiting the symptoms of the disease or condition either prophylactically and/or therapeutically.

As described herein, the term “percent (%) sequence identity,” and terms related thereto, in the context of amino acid sequences, is the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as Clustal Omega, BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared.

The terms “polypeptide” and “protein” are used interchangeably herein to refer to a polymer of amino acid residues. Peptides also include essentially any polyamino acid including, but not limited to, peptide mimetics such as amino acids joined by an ether as opposed to an amide bond.

### Payloads and Delivery Constructs

Provided herein are delivery constructs capable of transporting one or more heterologous payload molecules (e.g., one or more therapeutic payloads) across epithelial cells (e.g., polarized gut epithelial cells) and into the lamina propria via transcytosis. The delivery constructs can comprise a carrier coupled to a heterologous payload. The delivery construct can be a non-naturally occurring fusion protein. The carrier can be capable of transporting the heterologous payload across polarized epithelial cells (e.g., polarized gut epithelial cells) using endogenous trafficking pathways. Utilization of endogenous trafficking pathways, as opposed to use of passive diffusion, can allow the carrier to shuttle the heterologous payload rapidly and efficiently across epithelial cells without impairing the barrier function of these cells or the biological activity of the heterologous payload. Further provided herein are methods for purification and refolding of the delivery constructs described herein.

A carrier herein may be derived from a polypeptide secreted by a bacterium. Such carrier may be derived from a polypeptide secreted from Vibrio cholerae or Pseudomonas aeruginosa. The polypeptide secreted by Vibrio cholerae can be a Cholix polypeptide. A carrier derived from a Cholix polypeptide can be naturally occurring or non-naturally occurring. For example, a non-naturally occurring Cholix polypeptide can consist of the amino acid sequence set forth in SEQ ID NO: 1 (an example of a Cholix¹⁻⁶³⁴) (TABLE 1). A carrier derived from a Cholix polypeptide can be a truncated and/or mutated variant of a polypeptide derived from Cholix. For example, the carrier can comprise or consist of an amino acid sequence of those with amino acid residues 1-206, 1-245, 1-251, 1-266, and 1-386 of SEQ ID NO: 1 or SEQ ID NO: 26. In some instances, such carriers have an amino acid sequence of those with amino acid residues 1-206, 1-245, 1-251, 1-266, and 1-386 of SEQ ID NO: 4. Mutation(s) can include one or more substitution(s), deletion(s), and/or addition(s). For example, a carrier herein can comprise a V1L substitution. Stated differently, in some embodiments, the cholix-related carrier has a leucine amino acid at position “1.” (Position 1 refers to the first amino acid of variants that do not have an N-terminal methionine or the second position in variants that include an N-terminal methionine. In other words, in determining the length of a carrier, an N-terminal methionine, if present, is ignored.) In some embodiments, carriers comprising the V1L substitution experience reduced or eliminated cleavage of the N-terminal amino acid. In some embodiments, carriers comprising the V1L substitution experience reduced or eliminated acetylation of the N-terminal amino acid. A carrier provided herein can have a reduced (e.g., at least 50% reduced) or ablated ADP ribosylation activity (e.g., ribosylation of elongation factor 2) relative to a naturally-occurring Cholix variant. In some embodiments, the carrier can comprise an N-terminal methionine. In other embodiments, no N-terminal methionine is present.

A truncated Cholix carrier can consist of, consist essentially of, or comprise amino acid residues 1-386 of a sequence set forth in SEQ ID NO: 26 (FORMULA I). A truncated Cholix carrier can consist of, consist essentially of, or comprise amino acid residues 1-266 of a sequence set forth in SEQ ID NO: 26 (FORMULA I). In such instances, a carrier can consist of, consist essentially of, or comprise amino acid residues 1-266 of a sequence set forth in SEQ ID NO: 1. Thus, in some instances, the carrier consists of the amino acid sequence set forth in SEQ ID NO: 2 (an example of Cholix¹⁻³⁸⁶) or SEQ ID NO: 3 (an example of Cholix¹⁻²⁶⁶). In some instances, a carrier has the amino acid sequence represented by SEQ ID NO: 2 with a V1L substitution. Thus, in some instances, the carrier consists of the amino acid sequence set forth in SEQ ID NO: 4 (an example of V1L-Cholix¹⁻³⁸⁶) or SEQ ID NO: 5 (an example of V1L Cholix¹⁻²⁶⁶). Any of these carriers can include one or more amino acids at its N-terminus for expression in various microorganisms (e.g., bacteria), e.g., an N-terminal methionine. Such carrier can have an amino acid sequence set forth in SEQ ID NOS: 6-9.

The Cholix polypeptide can be a protein comprising, consisting essentially of, or consisting of an amino acid sequence having at least 80%, 85%. 90%, 95%, 98%, or 99% sequence identity, or having 100% sequence identity, to an amino acid sequence set forth in any one of SEQ ID NOS: 1-9 or SEQ ID NOS: 22-23. An example of a Cholix polypeptide is provided herein as SEQ ID NO: 1. or SEQ ID NO: 22. Also contemplated herein are truncated Cholix polypeptide variants that are able to transport a payload across polarized epithelia cells (e.g., polarized gut epithelial cells). Such Cholix polypeptides can be truncated at any one of the amino acid positions from 206 to 633 as compared to a reference sequence, e.g. SEQ ID NO: 1, SEQ ID NO: 22, or SEQ ID NO: 26, or an amino acid sequence having at least 80%, 85%, 90%, 95%, 98% or 99% sequence identity thereto.

Also contemplated herein are delivery constructs comprising carriers having high sequence identity to the sequences above. Such high sequence identity can include, at least 90%, 95%, 96%, 97%, 98% or 99% sequence identity. Thus, in some instances, the carrier comprises a sequence identify of at least 90%, 95%, 96%, 97%, 98% or 99% sequence identity to an amino acid sequence set forth in SEQ ID NO: 6-9.

A carrier contemplated herein can be coupled to a payload, such as a heterologous payload. Such payload can be a therapeutic payload. A therapeutic payload can be a cytokine, a hormone, a growth factor, a therapeutic antibody, an antigen, a functional fragment of any of the above, or any other protein that has biological, therapeutic activity, or a protein that may be deficient in a subject (e.g., a genetic/inherited deficiency of a certain protein). Cytokines contemplated herein include monomeric chemokines and interleukins (also abbreviated herein as “ILs”). The interleukin can be IL-22. The interleukin may be from any species (e.g., from a human or a rodent). The interleukin may be a human interleukin. Human IL-22 can have the amino acid sequence set forth in SEQ ID NO: 10 (IL-22¹⁻¹⁷⁹) or SEQ ID NO: 11 (IL-22³⁴⁻¹⁷⁹). An IL-22 herein can further include a methionine at its N-terminus, e.g., when such IL-22 protein is bacterially expressed. In one instance, an IL-22 has an amino acid sequence set forth in SEQ ID NO: 12 (M+IL-22³⁴⁻¹⁷⁹). An IL-22 herein can further include a methionine at its N-terminus, e.g., when such IL-22 protein is bacterially expressed. In one instance, an IL-22 has an amino acid sequence set forth in SEQ ID NO: 12 (M+IL-22³⁴⁻¹⁷⁹). The IL-22 can comprise, consist essentially of, or consist of SEQ ID NO: 10, or an amino acid sequence having at least 80%, 85%, 90%, 95%, 98% or 99%% sequence identity thereto or fragment thereof. The IL-22 can comprise, consists essentially of, or consist of SEQ ID NO: 11 (IL-22³⁴⁻¹⁷⁹), which is a secreted form of IL-22, or an amino acid sequence having at least 80%, 85%, 90%, 95%, 98% or 99%% sequence identity thereto or fragment thereof. An IL-22 can include a methionine at its N-terminus, e.g., when such IL-22 protein is bacterially expressed. Such IL-22 can consist of, consist essentially of, or comprise an amino acid sequence set forth in SEQ ID NO: 12 (M+IL-22³⁴⁻¹⁷⁹).

In some instances, a carrier used in any of the delivery constructs herein can be a protein or another type of molecule capable of transporting the therapeutic payload across an epithelium (e.g., a polarized gut epithelium of a subject). Such transport can include transcytosis. As referred to herein, “transcytosis” refers to the trafficking of the fusion molecule through a polarized epithelial cell. Such trafficking permits the release of the biologically active cargo from the basolateral membrane of the polarized epithelial cell. The transcytosis process may involve interaction(s) of the carrier with one or more receptor(s) and/or protein(s) on the apical and/or basal surface(s) as well as inside a cell of the epithelium (e.g., a polarized gut epithelial cell). The carrier can be capable of transporting the therapeutic payload across an epithelium without impairing the epithelium, the carrier, and/or the biological and/or therapeutic function of the payload.

A carrier can be coupled to a therapeutic payload covalently or non-covalently and directly or indirectly. The therapeutic payload can be directly coupled to the N-terminus or C-terminus of the carrier. In instances where the carrier is covalently coupled to the payload, the carrier can be coupled to such payload via a spacer (also referred to herein as a linker). The spacer can comprise at least 1, 2, 3, 4, 5, 6, 7, 8, 9, 10, 15, 20, or more amino acid residues. Spacer amino acid residues can be e.g., glycine, serine, and/or tryptophan. A spacer may be a cleavable or a non-cleavable spacer. A cleavable spacer may be cleavable by an enzyme or in a pH-dependent manner.

Examples of spacers contemplated herein include oligopeptide sequences such as S, (GS)x, (GGS)x, (GGGS)x (SEQ ID NO: 27), (GGGGS)x (SEQ ID NO: 28), or (GGGGGS)x (SEQ ID NO: 29), wherein x=1, 2, 3, 4, 5, 6, 7, 8, 9, 10, 11, 12, 13, 14, or 15. In some cases, a spacer consists of, consists essentially of, or comprises a sequence set forth in SEQ ID NO: 13 ((G₄S)₃). (GGGGSGGGGSGGGGS). In some cases, a spacer consists of, consists essentially of, or comprises a sequence set forth in SEQ ID NO: 31 ((G₄S)₅). (GGGGSGGGGSGGGGSGGGGSGGGGS). In some instances, a delivery construct comprises a therapeutic payload and a carrier that are non-covalently linked (e.g., via ionic interactions, van der Waals interactions, π-π interactions, etc.). A carrier can further comprise one or more features, elements, amino acids, or modifications on its N-terminus and/or C-terminus. For instance, some embodiments include a N-terminal methionine at the N-terminus of the carrier. Other modifications (e.g., acetylation) may also be present, including modifications for expression in a heterologous system.

A delivery construct herein can have an amino acid sequence set forth in SEQ ID NO: 15 or 17 (examples of M+Cholix¹⁻²⁶⁶-(G₄S)₃-IL-22³⁴⁻¹⁷⁹) (TABLE 1). Other delivery constructs can have the amino acid sequence set forth in SEQ ID NOs: 24 or 25 (e.g., when expressed in a mammalian cell such as CHO cell). Such exemplary delivery constructs transport IL-22 across intact, polarized gut epithelial cells and into the lamina propria.

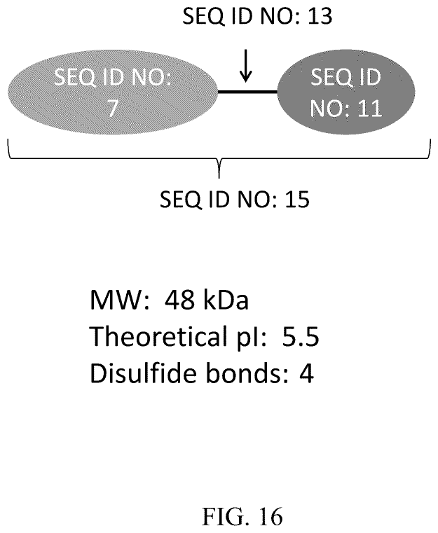

In various embodiments, the non-naturally occurring fusion protein has an amino acid sequence set forth in SEQ ID NO: 14-21, 24, or 25, or an amino acid sequence having at least 80%, 85%, 90%, 95%, 98% or 99% sequence identity thereto. A schematic of the carrier, spacer, and payload of a non-naturally occurring fusion protein comprising an amino acid sequence set forth in SEQ ID NO: 15 is illustrated in FIG. 16.

TABLE 1 shows exemplary amino acid sequences of biologically active molecules used in combination with the herein disclosed methods.

TABLE 1
Exemplary Amino Acid Sequences

| Category | SEQ ID NO | Amino Acid Sequence |
|---|---|---|
| Cholix¹⁻⁶³⁴ | SEQ ID NO: | VEEALNIFDECRSPCSLTPEPGKPIQSKLSIPSDVVLD |
| | 1 | EGVLYYSMTINDEQNDIKDEDKGESIITIGEFATVRA |
| | | TRHYVNQDAPFGVIHLDITTENGTKTYSYNRKEGEF |
| | | AINWLVPIGEDSPASIKISVDELDQQRNIIEVPKLYSI |
| | | DLDNQTLEQWKTQGNVSFSVTRPEHNIAISWPSVS |
| | | YKAAQKEGSRHKRWAHWHTGLALCWLVPMDAIY |
| | | NYITQQNCTLGDNWFGGSYETVAGTPKVITVKQGI |
| | | EQKPVEQRIHFSKGNAMSALAAHRVCGVPLETLAR |
| | | SRKPRDLTDDLSCAYQAQNIVSLFVATRILFSHLDS |
| | | VFTLNLDEQEPEVAERLSDLRRINENNPGMVTQVL |
| | | TVARQIYNDYVTHHPGLTPEQTSAGAQAADILSLFC |
| | | PDADKSCVASNNDQANINIESRSGRSYLPENRAVIT |
| | | PQGVTNWTYQELEATHQALTREGYVFVGYHGTNH |
| | | VAAQTIVNRIAPVPRGNNTENEEKWGGLYVATHAE |
| | | VAHGYARIKEGTGEYGLPTRAERDARGVMLRVYIP |
| | | RASLERFYRTNTPLENAEEHITQVIGHSLPLRNEAFT |
| | | GPESAGGEDETVIGWDMAIHAVAIPSTIPGNAYEEL |
| | | AIDEEAVAKEQSISTKPPYKERKDELK |
| Cholix¹⁻³⁸⁶ | SEQ ID NO: | VEEALNIFDECRSPCSLTPEPGKPIQSKLSIPSDVVLD |
| | 2 | EGVLYYSMTINDEQNDIKDEDKGESIITIGEFATVRA |
| | | TRHYVNQDAPFGVIHLDITTENGTKTYSYNRKEGEF |
| | | AINWLVPIGEDSPASIKISVDELDQQRNIIEVPKLYSI |
| | | DLDNQTLEQWKTQGNVSFSVTRPEHNIAISWPSVS |
| | | YKAAQKEGSRHKRWAHWHTGLALCWLVPMDAIY |
| | | NYITQQNCTLGDNWFGGSYETVAGTPKVITVKQGI |
| | | EQKPVEQRIHFSKGNAMSALAAHRVCGVPLETLAR |
| | | SRKPRDLTDDLSCAYQAQNIVSLFVATRILFSHLDS |
| | | VFTLNLDEQEPEVAERLSDLRRINENNPGMVTQVL |
| | | TVARQIYNDYVTHHPGLTPEQTSAGAQA |
| Cholix¹⁻²⁶⁶ | SEQ ID NO: | VEEALNIFDECRSPCSLTPEPGKPIQSKLSIPSDVVLD |
| | 3 | EGVLYYSMTINDEQNDIKDEDKGESIITIGEFATVRA |
| | | TRHYVNQDAPFGVIHLDITTENGTKTYSYNRKEGEF |
| | | AINWLVPIGEDSPASIKISVDELDQQRNIIEVPKLYSI |
| | | DLDNQTLEQWKTQGNVSFSVTRPEHNIAISWPSVS |
| | | YKAAQKEGSRHKRWAHWHTGLALCWLVPMDAIY |
| | | NYITQQNCTLGDNWFGGSYETVAGTPKVITVKQGI |
| | | EQKPVEQRIHFSKG |
| (V1L)-Cholix¹⁻³⁸⁹ | SEQ ID NO: | LEEALNIFDECRSPCSLTPEPGKPIQSKLSIPSDVVLD |
| | 4 | EGVLYYSMTINDEQNDIKDEDKGESIITIGEFATVRA |
| | | TRHYVNQDAPFGVIHLDITTENGTKTYSYNRKEGEF |
| | | AINWLVPIGEDSPASIKISVDELDQQRNIIEVPKLYSI |
| | | DLDNQTLEQWKTQGNVSFSVTRPEHNIAISWPSVS |
| | | YKAAQKEGSRHKRWAHWHTGLALCWLVPMDAIY |
| | | NYITQQNCTLGDNWFGGSYETVAGTPKVITVKQGI |
| | | EQKPVEQRIHFSKGNAMSALAAHRVCGVPLETLAR |
| | | SRKPRDLTDDLSCAYQAQNIVSLFVATRILFSHLDS |
| | | VFTLNLDEQEPEVAERLSDLRRINENNPGMVTQVL |
| | | TVARQIYNDYVTHHPGLTPEQTSAGAQA |
| (V1L)-Cholix¹⁻²⁶⁶ | SEQ ID NO: | LEEALNIFDECRSPCSLTPEPGKPIQSKLSIPSDVVLD |
| | 5 | EGVLYYSMTINDEQNDIKDEDKGESIITIGEFATVRA |
| | | TRHYVNQDAPFGVIHLDITTENGTKTYSYNRKEGEF |
| | | AINWLVPIGEDSPASIKISVDELDQQRNIIEVPKLYSI |
| | | DLDNQTLEQWKTQGNVSFSVTRPEHNIAISWPSVS |
| | | YKAAQKEGSRHKRWAHWHTGLALCWLVPMDAIY |
| | | NYITQQNCTLGDNWFGGSYETVAGTPKVITVKQGI |
| | | EQKPVEQRIHFSKG |
| M+Cholix¹⁻³⁸⁶ | SEQ ID NO: | MVEEALNIFDECRSPCSLTPEPGKPIQSKLSIPSDVVL |
| | 6 | DEGVLYYSMTINDEQNDIKDEDKGESIITIGEFATVR |
| | | ATRHYVNQDAPFGVIHLDITTENGTKTYSYNRKEG |
| | | EFAINWLVPIGEDSPASIKISVDELDQQRNIIEVPKLY |
| | | SIDLDNQTLEQWKTQGNVSFSVTRPEHNIAISWPSV |
| | | SYKAAQKEGSRHKRWAHWHTGLALCWLVPMDAI |
| | | YNYITQQNCTLGDNWFGGSYETVAGTPKVITVKQG |
| | | IEQKPVEQRIHFSKGNAMSALAAHRVCGVPLETLA |
| | | RSRKPRDLTDDLSCAYQAQNIVSLFVATRILFSHLD |
| | | SVFTLNLDEQEPEVAERLSDLRRINENNPGMVTQVL |
| | | TVARQIYNDYVTHHPGLTPEQTSAGAQA |
| M+Cholix¹⁻²⁶⁶ | SEQ ID NO: | MVEEALNIFDECRSPCSLTPEPGKPIQSKLSIPSDVVL |
| | 7 | DEGVLYYSMTINDEQNDIKDEDKGESIITIGEFATVR |
| | | ATRHYVNQDAPFGVIHLDITTENGTKTYSYNRKEG |
| | | EFAINWLVPIGEDSPASIKISVDELDQQRNIIEVPKLY |
| | | SIDLDNQTLEQWKTQGNVSFSVTRPEHNIAISWPSV |
| | | SYKAAQKEGSRHKRWAHWHTGLALCWLVPMDAI |
| | | YNYITQQNCTLGDNWFGGSYETVAGTPKVITVKQG |
| | | IEQKPVEQRIHFSKG |
| M+(V1L)- | SEQ ID NO: | MLEEALNIFDECRSPCSLTPEPGKPIQSKLSIPSDVVL |
| Cholix¹⁻³⁸⁶ | 8 | DEGVLYYSMTINDEQNDIKDEDKGESIITIGEFATVR |
| | | ATRHYVNQDAPFGVIHLDITTENGTKTYSYNRKEG |
| | | EFAINWLVPIGEDSPASIKISVDELDQQRNIIEVPKLY |
| | | SIDLDNQTLEQWKTQGNVSFSVTRPEHNIAISWPSV |
| | | SYKAAQKEGSRHKRWAHWHTGLALCWLVPMDAI |
| | | YNYITQQNCTLGDNWFGGSYETVAGTPKVITVKQG |
| | | IEQKPVEQRIHFSKGNAMSALAAHRVCGVPLETLA |
| | | RSRKPRDLTDDLSCAYQAQNIVSLFVATRILFSHLD |
| | | SVFTLNLDEQEPEVAERLSDLRRINENNPGMVTQVL |
| | | TVARQIYNDYVTHHPGLTPEQTSAGAQA |
| M+(V1L)- | SEQ ID NO: | MLEEALNIFDECRSPCSLTPEPGKPIQSKLSIPSDVVL |
| Cholix¹⁻²⁶⁶ | 9 | DEGVLYYSMTINDEQNDIKDEDKGESIITIGEFATVR |
| | | ATRHYVNQDAPFGVIHLDITTENGTKTYSYNRKEG |
| | | EFAINWLVPIGEDSPASIKISVDELDQQRNIIEVPKLY |
| | | SIDLDNQTLEQWKTQGNVSFSVTRPEHNIAISWPSV |
| | | SYKAAQKEGSRHKRWAHWHTGLALCWLVPMDAI |
| | | YNYITQQNCTLGDNWFGGSYETVAGTPKVITVKQG |
| | | IEQKPVEQRIHFSKG |
| IL-22¹⁻¹⁷⁹ | SEQ ID NO: | MAALQKSVSSFLMGTLATSCLLLLALLVQGGAAAP |
| | 10 | ISSHCRLDKSNFQQPYITNRTFMLAKEASLADNNTD |
| | | VRLIGEKLFHGVSMSERCYLMKQVLNFTLEEVLFP |
| | | QSDRFQPYMQEVVPFLARLSNRLSTCHIEGDDLHIQ |
| | | RNVQKLKDTVKKLGESGEIKAIGELDLLFMSLRNA |
| | | CI |
| IL-22³⁴⁻¹⁷⁹ | SEQ ID NO: | APISSHCRLDKSNFQQPYITNRTFMLAKEASLADNN |
| | 11 | TDVRLIGEKLFHGVSMSERCYLMKQVLNFTLEEVL |
| | | FPQSDRFQPYMQEVVPFLARLSNRLSTCHIEGDDLH |
| | | IQRNVQKLKDTVKKLGESGEIKAIGELDLLFMSLRN |
| | | ACI |
| M+IL-22³⁴⁻¹⁷⁹ | SEQ ID NO: | MAPISSHCRLDKSNFQQPYITNRTFMLAKEASLADN |
| | 12 | NTDVRLIGEKLFHGVSMSERCYLMKQVLNFTLEEV |
| | | LFPQSDRFQPYMQEVVPFLARLSNRLSTCHIEGDDL |
| | | HIQRNVQKLKDTVKKLGESGEIKAIGELDLLFMSLR |
| | | NACI |
| (G₄S)₃ | SEQ ID NO: | GGGGSGGGGSGGGGS |
| | 13 | |
| M+Cholix¹⁻³⁸⁶- | SEQ ID NO: | MLEEALNIFDECRSPCSLTPEPGKPIQSKLSIPSDVVL |
| (G₄S)₃-IL-22³⁴⁻¹⁷⁹ | 14 | DEGVLYYSMTINDEQNDIKDEDKGESIITIGEFATVR |
| | | ATRHYVNQDAPFGVIHLDITTENGTKTYSYNRKEG |
| | | EFAINWLVPIGEDSPASIKISVDELDQQRNIIEVPKLY |
| | | SIDLDNQTLEQWKTQGNVSFSVTRPEHNIAISWPSV |
| | | SYKAAQKEGSRHKRWAHWHTGLALCWLVPMDAI |
| | | YNYITQQNCTLGDNWFGGSYETVAGTPKVITVKQG |
| | | IEQKPVEQRIHFSKGNAMSALAAHRVCGVPLETLA |
| | | RSRKPRDLTDDLSCAYQAQNIVSLFVATRILFSHLD |
| | | SVFTLNLDEQEPEVAERLSDLRRINENNPGMVTQVL |
| | | TVARQIYNDYVTHHPGLTPEQTSAGAQAGGGGSG |
| | | GGGSGGGGSAPISSHCRLDKSNFQQPYITNRTFMLA |
| | | KEASLADNNTDVRLIGEKLFHGVSMSERCYLMKQ |
| | | VLNFTLEEVLFPQSDRFQPYMQEVVPFLARLSNRLS |
| | | TCHIEGDDLHIQRNVQKLKDTVKKLGESGEIKAIGE |
| | | LDLLFMSLRNACI |
| M+Cholix¹⁻²⁶⁶- | SEQ ID NO: | MVEEALNIFDECRSPCSLTPEPGKPIQSKLSIPSDVVL |
| (G₄S)₃-IL-22³⁴⁻¹⁷⁹ | 15 | DEGVLYYSMTINDEQNDIKDEDKGESIITIGEFATVR |
| | | ATRHYVNQDAPFGVIHLDITTENGTKTYSYNRKEG |
| | | EFAINWLVPIGEDSPASIKISVDELDQQRNIIEVPKLY |
| | | SIDLDNQTLEQWKTQGNVSFSVTRPEHNIAISWPSV |
| | | SYKAAQKEGSRHKRWAHWHTGLALCWLVPMDAI |
| | | YNYITQQNCTLGDNWFGGSYETVAGTPKVIT VKQG |
| | | IEQKPVEQRIHFSKGGGGGSGGGGSGGGGSAPISSH |
| | | CRLDKSNFQQPYITNRTFMLAKEASLADNNTDVRLI |
| | | GEKLFHGVSMSERCYLMKQVLNFTLEEVLFPQSDR |
| | | FQPYMQEVVPFLARLSNRLSTCHIEGDDLHIQRNVQ |
| | | KLKDTVKKLGESGEIKAIGELDLLFMSLRNACI |
| M+(V1L)- | SEQ ID NO: | MVEEALNIFDECRSPCSLTPEPGKPIQSKLSIPSDVVL |
| Cholix¹⁻³⁸⁶- | 16 | DEGVLYYSMTINDEQNDIKDEDKGESIITIGEFATVR |
| (G₄S)₃-IL-22³⁴⁻¹⁷⁹ | | ATRHYVNQDAPFGVIHLDITTENGTKTYSYNRKEG |
| | | EFAINWLVPIGEDSPASIKISVDELDQQRNIIEVPKLY |
| | | SIDLDNQTLEQWKTQGNVSFSVTRPEHNIAISWPSV |
| | | SYKAAQKEGSRHKRWAHWHTGLALCWLVPMDAI |
| | | YNYITQQNCTLGDNWFGGSYETVAGTPKVITVKQG |
| | | IEQKPVEQRIHFSKGNAMSALAAHRVCGVPLETLA |
| | | RSRKPRDLTDDLSCAYQAQNIVSLFVATRILFSHLD |
| | | SVFTLNLDEQEPEVAERLSDLRRINENNPGMVTQVL |
| | | TVARQIYNDYVTHHPGLTPEQTSAGAQAGGGGSG |
| | | GGGSGGGGSAPISSHCRLDKSNFQQPYITNRTFMLA |
| | | KEASLADNNTDVRLIGEKLFHGVSMSERCYLMKQ |
| | | VLNFTLEEVLFPQSDRFQPYMQEVVPFLARLSNRLS |
| | | TCHIEGDDLHIQRNVQKLKDTVKKLGESGEIKAIGE |
| | | LDLLFMSLRNACI |
| M+(V1L)- | SEQ ID NO: | MLEEALNIFDECRSPCSLTPEPGKPIQSKLSIPSDVVL |
| Cholix¹⁻²⁶⁶- | 17 | DEGVLYYSMTINDEQNDIKDEDKGESIITIGEFATVR |
| (G₄S)₃-IL-22³⁴⁻¹⁷⁹ | | ATRHYVNQDAPFGVIHLDITTENGTKTYSYNRKEG |
| | | EFAINWLVPIGEDSPASIKISVDELDQQRNIIEVPKLY |
| | | SIDLDNQTLEQWKTQGNVSFSVTRPEHNIAISWPSV |
| | | SYKAAQKEGSRHKRWAHWHTGLALCWLVPMDAI |
| | | YNYITQQNCTLGDNWFGGSYETVAGTPKVITVKQG |
| | | IEQKPVEQRIHFSKGGGGGSGGGGSGGGGSAPISSH |
| | | CRLDKSNFQQPYITNRTFMLAKEASLADNNTDVRLI |
| | | GEKLFHGVSMSERCYLMKQVLNFTLEEVLFPQSDR |
| | | FQPYMQEVVPFLARLSNRLSTCHIEGDDLHIQRNVQ |
| | | KLKDTVKKLGESGEIKAIGELDLLFMSLRNACI |
| Cholix¹⁻³⁸⁶- | SEQ ID NO: | VEEALNIFDECRSPCSLTPEPGKPIQSKLSIPSDVVLD |
| (G₄S)₃-IL-22³⁴⁻¹⁷⁹ | 18 | EGVLYYSMTINDEQNDIKDEDKGESIITIGEFATVRA |
| | | TRHYVNQDAPFGVIHLDITTENGTKTYSYNRKEGEF |
| | | AINWLVPIGEDSPASIKISVDELDQQRNIIEVPKLYSI |
| | | DLDNQTLEQWKTQGNVSFSVTRPEHNIAISWPSVS |
| | | YKAAQKEGSRHKRWAHWHTGLALCWLVPMDAIY |
| | | NYITQQNCTLGDNWFGGSYETVAGTPKVITVKQGI |
| | | EQKPVEQRIHFSKGNAMSALAAHRVCGVPLETLAR |
| | | SRKPRDLTDDLSCAYQAQNIVSLFVATRILFSHLDS |
| | | VFTLNLDEQEPEVAERLSDLRRINENNPGMVTQVL |
| | | TVARQIYNDYVTHHPGLTPEQTSAGAQAGGGGSG |
| | | GGGSGGGGSAPISSHCRLDKSNFQQPYITNRTFMLA |
| | | KEASLADNNTDVRLIGEKLFHGVSMSERCYLMKQ |
| | | VLNFTLEEVLFPQSDRFQPYMQEVVPFLARLSNRLS |
| | | TCHIEGDDLHIQRNVQKLKDTVKKLGESGEIKAIGE |
| | | LDLLFMSLRNACI |
| (V1L)-Cholix¹⁻³⁸⁶- | SEQ ID NO: | LEEALNIFDECRSPCSLTPEPGKPIQSKLSIPSDVVLD |
| (G₄S)₃-IL-22³⁴⁻¹⁷⁹ | 19 | EGVLYYSMTINDEQNDIKDEDKGESIITIGEFATVRA |
| | | TRHYVNQDAPFGVIHLDITTENGTKTYSYNRKEGEF |
| | | AINWLVPIGEDSPASIKISVDELDQQRNIIEVPKLYSI |
| | | DLDNQTLEQWKTQGNVSFSVTRPEHNIAISWPSVS |
| | | YKAAQKEGSRHKRWAHWHTGLALCWLVPMDAIY |
| | | NYITQQNCTLGDNWFGGSYETVAGTPKVITVKQGI |
| | | EQKPVEQRIHFSKGNAMSALAAHRVCGVPLETLAR |
| | | SRKPRDLTDDLSCAYQAQNIVSLFVATRILFSHLDS |
| | | VFTLNLDEQEPEVAERLSDLRRINENNPGMVTQVL |
| | | TVARQIYNDYVTHHPGLTPEQTSAGAQAGGGGSG |
| | | GGGSGGGGSAPISSHCRLDKSNFQQPYITNRTFMLA |
| | | KEASLADNNTDVRLIGEKLFHGVSMSERCYLMKQ |
| | | VLNFTLEEVLFPQSDRFQPYMQEVVPFLARLSNRLS |
| | | TCHIEGDDLHIQRNVQKLKDTVKKLGESGEIKAIGE |
| | | LDLLFMSLRNACI |
| Cholix¹⁻²⁶⁶- | SEQ ID NO: | VEEALNIFDECRSPCSLTPEPGKPIQSKLSIPSDVVLD |
| (G₄S)₃-IL-22³⁴⁻¹⁷⁹ | 20 | EGVLYYSMTINDEQNDIKDEDKGESIITIGEFATVRA |
| | | TRHYVNQDAPFGVIHLDITTENGTKTYSYNRKEGEF |
| | | AINWLVPIGEDSPASIKISVDELDQQRNIIEVPKLYSI |
| | | DLDNQTLEQWKTQGNVSFSVTRPEHNIAISWPSVS |
| | | YKAAQKEGSRHKRWAHWHTGLALCWLVPMDAIY |
| | | NYITQQNCTLGDNWFGGSYETVAGTPKVITVKQGI |
| | | EQKPVEQRIHFSKGGGGGSGGGGSGGGGSAPISSHC |
| | | RLDKSNFQQPYITNRTFMLAKEASLADNNTDVRLI |
| | | GEKLFHGVSMSERCYLMKQVLNFTLEEVLFPQ SDR |
| | | FQPYMQEVVPFLARLSNRLSTCHIEGDDLHIQRNVQ |
| | | KLKDTVKKLGESGEIKAIGELDLLFMSLRNACI |
| (V1L)-Cholix¹⁻²⁶⁶- | SEQ ID NO: | LEEALNIFDECRSPCSLTPEPGKPIQSKLSIPSDVVLD |
| (G₄S)₃-IL-22³⁴⁻¹⁷⁹ | 21 | EGVLYYSMTINDEQNDIKDEDKGESIITIGEFATVRA |
| | | TRHYVNQDAPFGVIHLDITTENGTKTYSYNRKEGEF |
| | | AINWLVPIGEDSPASIKISVDELDQQRNIIEVPKLYSI |
| | | DLDNQTLEQWKTQGNVSFSVTRPEHNIAISWPSVS |
| | | YKAAQKEGSRHKRWAHWHTGLALCWLVPMDAIY |
| | | NYITQQNCTLGDNWFGGSYETVAGTPKVITVKQGI |
| | | EQKPVEQRIHFSKGGGGGSGGGGSGGGGSAPISSHC |
| | | RLDKSNFQQPYITNRTFMLAKEASLADNNTDVRLI |
| | | GEKLFHGVSMSERCYLMKQVLNFTLEEVLFPQSDR |
| | | FQPYMQEVVPFLARLSNRLSTCHIEGDDLHIQRNVQ |
| | | KLKDTVKKLGESGEIKAIGELDLLFMSLRNACI |
| (V1L)-Cholix¹⁻⁶³⁴ | SEQ ID NO: | LEEALNIFDECRSPCSLTPEPGKPIQSKLSIPSDVVLD |
| | 22 | EGVLYYSMTINDEQNDIKDEDKGESIITIGEFATVRA |
| | | TRHYVNQDAPFGVIHLDITTENGTKTYSYNRKEGEF |
| | | AINWLVPIGEDSPASIKISVDELDQQRNIIEVPKLYSI |
| | | DLDNQTLEQWKTQGNVSFSVTRPEHNIAISWPSVS |
| | | YKAAQKEGSRHKRWAHWHTGLALCWLVPMDAIY |
| | | NYITQQNCTLGDNWFGGSYETVAGTPKVITVKQGI |
| | | EQKPVEQRIHFSKGNAMSALAAHRVCGVPLETLAR |
| | | SRKPRDLTDDLSCAYQAQNIVSLFVATRILFSHLDS |
| | | VFTLNLDEQEPEVAERLSDLRRINENNPGMVTQVL |
| | | TVARQIYNDYVTHHPGLTPEQTSAGAQAADILSLFC |
| | | PDADKSCVASNNDQANINIESRSGRSYLPENRAVIT |
| | | PQGVTNWTYQELEATHQALTREGYVFVGYHGTNH |
| | | VAAQTIVNRIAPVPRGNNTENEEKWGGLYVATHAE |
| | | VAHGYARIKEGTGEYGLPTRAERDARGVMLRVYIP |
| | | RASLERFYRTNTPLENAEEHITQVIGHSLPLRNEAFT |
| | | GPESAGGEDETVIGWDMAIHAVAIPSTIPGNAYEEL |
| | | AIDEEAVAKEQSISTKPPYKERKDELK |
| M+(V1L)- | SEQ ID NO: | MLEEALNIFDECRSPCSLTPEPGKPIQSKLSIPSDVVL |
| Cholix¹⁻⁶³⁴ | 23 | DEGVLYYSMTINDEQNDIKDEDKGESIITIGEFATVR |
| | | ATRHYVNQDAPFGVIHLDITTENGTKTYSYNRKEG |
| | | EFAINWLVPIGEDSPASIKISVDELDQQRNIIEVPKLY |
| | | SIDLDNQTLEQWKTQGNVSFSVTRPEHNIAISWPSV |
| | | SYKAAQKEGSRHKRWAHWHTGLALCWLVPMDAI |
| | | YNYITQQNCTLGDNWFGGSYETVAGTPKVITVKQG |
| | | IEQKPVEQRIHFSKGNAMSALAAHRVCGVPLETLA |
| | | RSRKPRDLTDDLSCAYQAQNIVSLFVATRILFSHLD |
| | | SVFTLNLDEQEPEVAERLSDLRRINENNPGMVTQVL |
| | | TVARQIYNDYVTHHPGLTPEQTSAGAQAADILSLFC |
| | | PDADKSCVASNNDQANINIESRSGRSYLPENRAVIT |
| | | PQGVTNWTYQELEATHQALTREGYVFVGYHGTNH |
| | | VAAQTIVNRIAPVPRGNNTENEEKWGGLYVATHAE |
| | | VAHGYARIKEGTGEYGLPTRAERDARGVMLRVYIP |
| | | RASLERFYRTNTPLENAEEHITQVIGHSLPLRNEAFT |
| | | GPESAGGEDETVIGWDMAIHAVAIPSTIPGNAYEEL |
| | | AIDEEAVAKEQSISTKPPYKERKDELK |
| Cholix¹⁻²⁶⁶- | SSEQ ID NO: | VEEALNIFDECRSPCSLTPEPGKPIQSKLSIPSDVVLD |
| (G₄S)₃-IL-22³⁴⁻¹⁷⁹ | 24 | TRHYVNQDAPFGVIHLDITTENGTKTYSYNRKEGEF |
| | | AINWLVPIGEDSPASIKISVDELDQQRNIIEVPKLYSI |
| | | DLDNQTLEQWKTQGNVSFSVTRPEHNIAISWPSVS |
| | | YKAAQKEGSRHKRWAHWHTGLALCWLVPMDAIY |
| | | NYITQQNCTLGDNWFGGSYETVAGTPKVITVKQGI |
| | | EQKPVEQRIHFSKGGGGGSGGGGSGGGGSAPISSHC |
| | | RLDKSNFQQPYITNRTFMLAKEASLADNNTDVRLI |
| | | GEKLFHGVSMSERCYLMKQVLNFTLEEVLFPQSDR |
| | | FQPYMQEVVPFLARLSNRLSTCHIEGDDLHIQRNVQ |
| | | KLKDTVKKLGESGEIKAIGELDLLFMSLRNACI |
| (V1L)-Cholix¹⁻²⁶⁶- | SSEQ ID | LEEALNIFDECRSPCSLTPEPGKPIQSKLSIPSDVVLD |
| (G₄S)₃-IL-22³⁴⁻¹⁷⁹ | NO: 25 | EGVLYYSMTINDEQNDIKDEDKGESITTIGEFATVRA |
| | | TRHYVNQDAPFGVIHLDITTENGTKTYSYNRKEGEF |
| | | AINWLVPIGEDSPASIKISVDELDQQRNIIEVPKLYSI |
| | | DLDNQTLEQWKTQGNVSFSVTRPEHNIAISWPSVS |
| | | YKAAQKEGSRHKRWAHWHTGLALCWLVPMDAIY |
| | | NYITQQNCTLGDNWFGGSYETVAGTPKVITVKQGI |
| | | EQKPVEQRIHFSKGGGGGSGGGGSGGGGSAPISSHC |
| | | RLDKSNFQQPYITNRTFMLAKEASLADNNTDVRLI |
| | | GEKLFHGVSMSERCYLMKQVLNFTLEEVLFPQSDR |
| | | FQPYMQEVVPFLARLSNRLSTCHIEGDDLHIQRNVQ |
| | | KLKDTVKKLGESGEIKAIGELDLLFMSLRNACI |
| M+Cholix¹⁻²⁶⁶- | SEQ ID NO: | MVEEALNIFDECRSPCSLTPEPGKPIQSKLSIPSDVVL |
| (G₄S)₁-IL-22³⁴⁻¹⁷⁹ | 32 | DEGVLYYSMTINDEQNDIKDEDKGESIITIGEFATVR |
| | | ATRHYVNQDAPFGVIHLDITTENGTKTYSYNRKEG |
| | | EFAINWLVPIGEDSPASIKISVDELDQQRNIIEVPKLY |
| | | SIDLDNQTLEQWKTQGNVSFSVTRPEHNIAISWPSV |
| | | SYKAAQKEGSRHKRWAHWHTGLALCWLVPMDAI |
| | | YNYITQQNCTLGDNWFGGSYETVAGTPKVITVKQG |
| | | IEQKPVEQRIFIFSKGGGGGSAPISSHCRLDKSNFQQP |
| | | YITNRTFMLAKEASLADNNTDVRLIGEKLFHGVSM |
| | | SERCYLMKQVLNFTLEEVLFPQSDRFQPYMQEVVP |
| | | FLARLSNRLSTCHIEGDDLHIQRNVQKLKDTVKKL |
| | | GESGEIKAIGELDLLFMSLRNACI |
| M+Cholix¹⁻²⁶⁶- | SEQ ID NO: | MVEEALNIFDECRSPCSLTPEPGKPIQSKLSIPSDVVL |
| (G₄S)₅-IL-22³⁴⁻¹⁷⁹ | 33 | DEGVLYYSMTINDEQNDIKDEDKGESIITIGEFATVR |
| | | ATRHYVNQDAPFGVIHLDITTENGTKTYSYNRKEG |
| | | EFAINWLVPIGEDSPASIKISVDELDQQRNIIEVPKLY |
| | | SIDLDNQTLEQWKTQGNVSFSVTRPEHNIAISWPSV |
| | | SYKAAQKEGSRHKRWAHWHTGLALCWLVPMDAI |
| | | YNYITQQNCTLGDNWFGGSYETVAGTPKVITVKQG |
| | | IEQKPVEQRIFIFSKGGGGGSGGGGSGGGGSGGGGS |
| | | GGGGSAPISSHCRLDKSNFQQPYITNRTFMLAKEAS |
| | | LADNNTDVRLIGEKLFHGVSMSERCYLMKQVLNFT |
| | | LEEVLFPQSDRFQPYMQEVVPFLARLSNRLSTCHIE |
| | | GDDLHIQRNVQKLKDTVKKLGESGEIKAIGELDLLF |
| | | MSLRNACI |
| M+IL-22³⁴⁻¹⁷⁹- | SEQ ID NO: | MAPISSHCRLDKSNFQQPYITNRTFMLAKEASLADN |
| (G₄S)₁-Cholix¹⁻²⁶⁶ | 34 | NTDVRLIGEKLEHGVSMSERCYLMKQVLNETLEEV |
| | | LFPQSDRFQPYMQEVVPFLARLSNRLSTCHIEGDDL |
| | | HIQRNVQKLKDTVKKLGESGEIKAIGELDLLFMSLR |
| | | NACIGGGGSVEEALNIFDECRSPCSLTPEPGKPIQSK |
| | | LSIPSDVVLDEGVLYYSMTINDEQNDIKDEDKGESII |
| | | TIGEFATVRATRHYVNQDAPFGVIHLDITTENGTKT |
| | | YSYNRKEGEFAINWLVPIGEDSPASIKISVDELDQQR |
| | | NIIEVPKLYSIDLDNQTLEQWKTQGNVSFSVTRPEH |
| | | NIAISWPSVSYKAAQKEGSRHKRWAHWHTGLALC |
| | | WLVPMDAIYNYITQQNCTLGDNWFGGSYETVAGT |
| | | PKVITVKQGIEQKPVEQRIHFSKG |
| M+Cholix¹⁻³⁸⁶- | SEQ ID NO: | MVEEALNIFDECRSPCSLTPEPGKPIQSKLSIPSDVVL |
| (G₄S)₁-IL-22³⁴⁻¹⁷⁹ | 35 | DEGVLYYSMTINDEQNDIKDEDKGESIITIGEFATVR |
| | | ATRHYVNQDAPFGVIHLDITTENGTKTYSYNRKEG |
| | | EFAINWLVPIGEDSPASIKISVDELDQQRNIIEVPKLY |
| | | SIDLDNQTLEQWKTQGNVSFSVTRPEHNIAISWPSV |
| | | SYKAAQKEGSRHKRWAHWHTGLALCWLVPMDAI |
| | | YNYITQQNCTLGDNWFGGSYETVAGTPKVITVKQG |
| | | IEQKPVEQRIHFSKGNAMSALAAHRVCGVPLETLA |
| | | RSRKPRDLTDDLSCAYQAQNIVSLFVATRILFSHLD |
| | | SVFTLNLDEQEPEVAERLSDLRRINENNPGMVTQVL |
| | | TVARQIYNDYVTHHPGLTPEQTSAGAQAGGGGSAP |
| | | ISSHCRLDKSNFQQPYITNRTFMLAKEASLADNNTD |
| | | VRLIGEKLFHGVSMSERCYLMKQVLNFTLEEVLFP |
| | | QSDRFQPYMQEVVPFLARLSNRLSTCHIEGDDLHIQ |
| | | RNVQKLKDTVKKLGESGEIKAIGELDLLFMSLRNA |
| | | CI |
| Cholix | SEQ ID NO: | X1-E-X3-X4-L-X6-I-F-D-E-C-R-S-P-C-X16-L-T-P-E- |
| Consensus | 26 | X21-G-K-X24-I-Q-S-K-L-X30-I-P-X33-D-V-V-L-D-E- |
| Sequence | | G-V-L-Y-Y-S-M-T-I-N-D-E-Q-N-D-I-X56-D-E-X59-K- |
| | | G-E-S-I-I-T-X67-G-E-F-A-T-X73-R-A-T-R-H-Y-V-X81- |
| | | Q-D-A-P-F-G-V-I-X90-L-D-I-T-T-E-N-G-T-K-X101-Y- |
| | | S-X104-N-R-K-X108-X109-E-F-X112-I-X114-W-L-V- |
| | | X118-X119-G-E-D-S-P-A-S-I-K-I-S-X131-D-E-X134-D- |
| | | Q-X137-R-N-I-I-E-V-P-K-L-Y-S-I-D-L-D-N-Q-T-L-E- |
| | | Q-W-X160-X161-Q-G-N-V-X166-F-X168-V-T-R-P-E- |
| | | X174-X175-I-A-I-S-W-P-S-V-S-Y-X186-A-A-X189-K- |
| | | X191-G-X193-R-H-K-R-W-A-X200-W-X202-T-X204- |
| | | X205-X206-X207-X208-X209-L-X211-X212-X213- |
| | | X214-X215-X216-X217-X218-X219-X220-X221-X222- |
| | | X223-X224-C-T-X227-G-X229-X230-W-X232-G-G- |
| | | X235-Y-X237-T-V-A-G-X242-P-X244-X245-I-X247-V- |
| | | K-Q-G-X252-E-Q-K-X256-V-E-Q-R-I-H-F-S-X265- |
| | | X266-N-A-X269-X270-X271-L-A-A-H-R-V-C-G-V-P-L- |
| | | E-T-L-A-R-X288-R-K-P-R-X293-L-X295-D-D-L-X299- |
| | | C-X301-Y-X303-A-Q-X306-I-V-S-L-F-X312-A-T-R- |
| | | X316-L-F-X319-H-X321-D-S-X324-F-T-L-N-L-X330- |
| | | X331-Q-X333-P-X335-V-X337-E-R-L-X341-X342- |
| | | X343-R-X345-I-N-E-X349-N-P-G-X353-V-X355-Q-V- |
| | | L-T-X360-A-R-Q-I-Y-N-D-Y-V-T-X371-H-P-X374-L- |
| | | X376-P-E-Q-T-S-A-X383-A-Q-A-A-D-I-L-S-L-X393- |
| | | X394-P-D-X397-D-X399-X400-C-V-A-X404-X405- |
| | | X406-D-Q-A-N-I-N-X413-E-S-R-S-G-R-S-Y-L-X423-E- |
| | | N-R-A-V-I-T-X431-Q-G-V-T-N-W-T-Y-Q-E-L-X443- |
| | | X444-X445-H-Q-X448-L-T-X451-E-X453-Y-V-F-V-G- |
| | | Y-H-G-T-N-H-X465-A-A-Q-X469-I-V-N-R-I-X475-P-V- |
| | | P-R-G-X481-X482-T-E-X485-E-X487-X488-W-G-G- |
| | | X492-Y-V-X495-T-X497-A-X499-X500-X501-X502- |
| | | X503-Y-X505-R-X507-X508-X509-G-T-X512-X513- |
| | | X514-X515-X516-X517-T-X519-X520-X521-X522- |
| | | X523-X524-R-G-V-M-L-X530-V-Y-X533-X534-X535- |
| | | A-S-L-E-R-F-Y-R-X544-N-X546-X547-L-E-X550- |
| | | X551-X552-X553-X554-X555-X556-X557-V-I-G-H- |
| | | X562-L-P-L-R-N-E-A-F-T-G-X573-X574-X575-X576- |
| | | X577-G-X579-X580-E-T-X583-I-G-W-D-X588-A-I- |
| | | X591-X592-V-A-I-P-S-T-I-P-G-N-X603-Y-X605-X606- |
| | | L-X608-X609-X610-E-E-A-X614-A-X616-E-Q-S-I-S- |
| | | X622-K-P-P-Y-K-E-X629-X630-D-E-L-K; wherein X1 is |
| | | selected from the group consisting of V and L; X3 is |
| | | selected from the group consisting of E and D; X4 is |
| | | selected from the group consisting of A and E; X6 is |
| | | selected from the group consisting of N and K; X16 is |
| | | selected from the group consisting of S and L; X21 is |
| | | selected from the group consisting of P and L; X24 is |
| | | selected from the group consisting of P and Q; X30 is |
| | | selected from the group consisting of S and F; X33 is |
| | | selected from the group consisting of S and G; X56 is |
| | | selected from the group consisting of K and M; X59 is |
| | | selected from the group consisting of D and G; X67 is |
| | | selected from the group consisting of I and F; X73 is |
| | | selected from the group consisting of V and I; X81 is |
| | | selected from the group consisting of N and S; X90 is |
| | | selected from the group consisting of H and N; X101 is |
| | | selected from the group consisting of T and M; X104 is |
| | | selected from the group consisting of Y and F; X108 is |
| | | selected from the group consisting of E and D; X109 is |
| | | selected from the group consisting of G and S; X112 is |
| | | selected from the group consisting of A and T; X114 is |
| | | selected from the group consisting of N and H; X118 is |
| | | selected from the group consisting of P and I; X119 is |
| | | selected from the group consisting of I and P; X131 is |
| | | selected from the group consisting of V and I; X134 is |
| | | selected from the group consisting L and I; X137 is |
| | | selected from the group consisting Q and K; X160 is |
| | | selected from the group consisting K and E; X161 is |
| | | selected from the group consisting T and N; X166 is |
| | | selected from the group consisting S and F; X168 is |
| | | selected from the group consisting S and A; X174 is |
| | | selected from the group consisting H and Q; X175 is |
| | | selected from the group consisting N, S, SIAKQS, and |
| | | SIAKQSIAKQS; X186 is selected from the group |
| | | consisting of K and N; X189 is selected from the group |
| | | consisting of Q, E, and H; X191 is selected from the |
| | | group consisting of E, N, and D; X193 is selected from |
| | | the group consisting of S and A; X200 is selected from the |
| | | group consisting of H and N; X202 is selected from the |
| | | group consisting of H, L, F, and R; X204 is selected from |
| | | the group consisting of G and T; X205 is selected from |
| | | the group consisting of L and S; X206 is selected from the |
| | | group consisting of A and P; X207 is selected from the |
| | | group consisting of L, E, and K; X208 is selected from the |
| | | group consisting of C and V; X209 is selected from the |
| | | group consisting of W, V, and T; X211 is selected from |
| | | the group consisting of V and no amino acid; X212 is |
| | | selected from the group consisting of P and no amino |
| | | acid; X213 is selected from the group consisting of M, I, |
| | | L, and no amino acid; X214 is selected from the group |
| | | consisting of D and no amino acid; X215 is selected from |
| | | the group consisting of A and no amino acid; X216 is |
| | | selected from the group consisting ofI and no amino acid; |
| | | X217 is selected from the group consisting of Y and C; |
| | | X218 is selected from the group consisting of N and F; |
| | | X219 is selected from the group consisting of Y and F; |
| | | X220 is selected from the group consisting of I and E; |
| | | X221 is selected from the group consisting of T and D; |
| | | X222 is selected from the group consisting of Q and P; |
| | | X223 is selected from the group consisting of Q, E, and |
| | | A; X224 is selected from the group consisting of N, L, |
| | | and Q; X227 is selected from the group consisting of L |
| | | and Y; X229 is selected from the group consisting of D |
| | | and E; X230 is selected from the group consisting of N |
| | | and D; X232 is selected from the group consisting of F, H, |
| | | and Y; X235 is selected from the group consisting of S |
| | | and A; X237 is selected from the group consisting of E |
| | | and K; X242 is selected from the group consisting of T |
| | | and I; X244 is selected from the group consisting of K, E, |
| | | and G; X245 is selected from the group consisting of V |
| | | and A; X247 is selected from the group consisting of T |
| | | and M; X252 is selected from the group consisting of I |
| | | and M; X256 is selected from the group consisting of P, |
| | | T, and A; X265 is selected from the group consisting of |
| | | K, Q, and N; X266 is selected from the group consisting |
| | | of G and K; X269 is selected from the group consisting of |
| | | M and I; X270 is selected from the group consisting of S |
| | | and E; X271 is selected from the group consisting of A |
| | | and T; X288 is selected from the group consisting of S |
| | | andG; X293 is selected from the group consisting of D |
| | | and Y; X295 is selected from the group consisting of T, P, |
| | | and Q; X299 is selected from the group consisting of S |
| | | and Q; X301 is selected from the group consisting of A |
| | | and V; X303 is selected from the group consisting of Q |
| | | and N; X306 is selected from the group consisting of N |
| | | and Q; X312 is selected from the group consisting of V |
| | | and L; X316 is selected from the group consisting of I and |
| | | M; X319 is selected from the group consisting of S and T; |
| | | X321 is selected from the group consisting of L and I; |
| | | X324 is selected from the group consisting of V and I; |
| | | X330 is selected from the group consisting of D, E, and |
| | | H; X331 is selected from the group consisting of E and G; |
| | | X333 is selected from the group consisting of E and A; |
| | | X335 is selected from the group consisting of E and A; |
| | | X337 is selected from the group consisting of A and T; |
| | | X341 is selected from the group consisting of S, D, and T; |
| | | X342 is selected from the group consisting of D and A; |
| | | X343 is selected from the group consisting of L and I; |
| | | X345 is selected from the group consisting of R and Q; |
| | | X349 is selected from the group consisting of N and D; |
| | | X353 is selected from the group consisting of M and V; |
| | | X355 is selected from the group consisting of T and I; |
| | | X360 is selected from the group consisting of V and I; |
| | | X371 is selected from the group consisting of H and E; |
| | | X374 is selected from the group consisting of G and L; |
| | | X376 is selected from the group consisting of T and I; |
| | | X383 is selected from the group consisting of G and S; |
| | | X393 is selected from the group consisting of F and L; |
| | | X394 is selected from the group consisting of C and Y; |
| | | X397 is selected from the group consisting of A and T; |
| | | X399 is selected from the group consisting of K, E, and |
| | | G; X400 is selected from the group consisting of S, P, and |
| | | H; X404 is selected from the group consisting of S and L; |
| | | X405 is selected from the group consisting of N and D; |
| | | X406 is selected from the group consisting of N and S; |
| | | X413 is selected from the group consisting of I and V; |
| | | X423 is selected from the group consisting of P and L; |
| | | X431 is selected from the group consisting of P and Q; |
| | | X443 is selected from the group consisting of E and |
| | | D; X444 is selected from the group consisting of A and T; |
| | | X445 is selected from the group consisting of T and K; |
| | | X448 is selected from the group consisting of A and T; |
| | | X451 is selected from the group consisting of R and Q; |
| | | X453 is selected from the group consisting of G and D; |
| | | X465 is selected from the group consisting of V and |
| | | A; X469 is selected from the group consisting of T, S, and |
| | | N; X475 is selected from the group consisting of A, S, and |
| | | T; X481 is selected from the group consisting of N and S; |
| | | X482 is selected from the group consisting of N and D; |
| | | X485 is selected from the group consisting of N, S, and K; |
| | | X487 is selected from the group consisting of E, R, and K; |
| | | X488 is selected from the group consisting of K, A, and |
| | | E; X492 is selected from the group consisting of L and V; |
| | | X495 is selected from the group consisting of A and S; |
| | | X497 is selected from the group consisting of H and D; |
| | | X499 is selected from the group consisting of E and S; |
| | | X500 is selected from the group consisting of V and L; |
| | | X501 is selected from the group consisting of A and N; |
| | | X502 is selected from the group consisting of H and Y; |
| | | X503 is selected from the group consisting of G and R; |
| | | X505 is selected from the group consisting of A and T; |
| | | X507 is selected from the group consisting of I and L; |
| | | X508 is selected from the group consisting of K and Q; |
| | | X509 is selected from the group consisting of E and K; |
| | | X512 is selected from the group consisting of G and A; |
| | | X513 is selected from the group consisting of E, D, and |
| | | N; X514 is selected from the group consisting of Y, G, A, |
| | | and N; X515 is selected from the group consisting of G |
| | | and E; X516 is selected from the group consisting of L |
| | | and G; X517 is selected from the group consisting of P |
| | | and L; X519 is selected from the group consisting of R, P, |
| | | and T; X520 is selected from the group consisting of A |
| | | and E; X521 is selected from the group consisting of E |
| | | and K; X522 is selected from the group consisting of R, |
| | | Q, and K; X523 is selected from the group consisting of |
| | | D, K, and E; X524 is selected from the group consisting |
| | | of A, T, and S; X530 is selected from the group consisting |
| | | of R and K; X533 is selected from the group consisting of |
| | | I and L; X534 is selected from the group consisting of P |
| | | and H; X535 is selected from the group consisting of R |
| | | and Q; X544 is selected from the group consisting of T |
| | | and I; X546 is selected from the group consisting of T, A, |
| | | and I; X547 is selected from the group consisting of P and |
| | | D; X550 is selected from the group consisting of N and K; |
| | | X551 is selected from the group consisting of A and E; |
| | | X552 is selected from the group consisting of E, R, and D; |
| | | X553 is selected from the group consisting of E, N, and R; |
| | | X554 is selected from the group consisting of H and L; |
| | | X555 is selected from the group consisting of I and V; |
| | | X556 is selected from the group consisting of T and E; |
| | | X557 is selected from the group consisting of Q, R, H, |
| | | and D; X562 is selected from the group consisting of S |
| | | and P; X573 is selected from the group consisting of P |
| | | and T; X574 is selected from the group consisting of E |
| | | and D; X575 is selected from the group consisting of S, A, |
| | | and R; X576 is selected from the group consisting of A, E, |
| | | and V; X577 is selected from the group consisting of G, |
| | | E, and D; X579 is selected from the group consisting of E |
| | | and S; X580 is selected from the group consisting of D |
| | | and N; X583 is selected from the group consisting of V |
| | | and A; X588 is selected from the group consisting of M |
| | | and I; X591 is selected from the group consisting of H and |
| | | Y; X592 is selected from the group consisting of A andG; |
| | | X603 is selected from the group consisting of A and S; |
| | | X605 is selected from the group consisting of E and A; |
| | | X606 is selected from the group consisting of E, A, Q, G, |
| | | V, and R; X608 is selected from the group consisting of |
| | | A, P, and T; X609 is selected from the group consisting of |
| | | I, T, and P; X610 is selected from the group consisting of |
| | | D and A; X614 is selected from the group consisting of V |
| | | and VVKEAI; X616 is selected from the group consisting |
| | | of K and E; X622 is selected from the group consisting of |
| | | T, A, and P; and X629 is selected from the group |
| | | consisting of R, Q, and H; and X630 is selected from the |
| | | group consisting of K and no amino acid. |

In some embodiments, the non-naturally occurring fusion protein comprises or consists of: a carrier selected from the group consisting of any one of the sequences set forth in SEQ ID NO: 1-9,22-23, and 26 and a payload selected from the group consisting of any one of the sequences set forth in SEQ ID NO: 10-12, and wherein the carrier and the payload are optionally coupled by a spacer selected from the group consisting of any one of the sequences set forth in SEQ ID NO: 13 and 27-29.

Further provided herein are pharmaceutical compositions comprising a delivery construct and one or more pharmaceutically acceptable carriers. In some cases, the delivery construct consists of the amino acid sequence set forth in SEQ ID NO: 15. In some cases, the delivery construct consists of the amino acid sequence set forth in SEQ ID NO: 17. Such pharmaceutical compositions can be formulated for administration to a subject. In some instances, a pharmaceutical composition is formulated for oral administration to a subject.

A pharmaceutical composition comprising a delivery construct can be administered to a subject (e.g., a human) in need thereof to treat a disease. Diseases that can be treated using the delivery constructs of this disclosure include autoimmune diseases and inflammatory diseases. In some instances, the disease is an epithelial cell injury or damage of epithelial cell membranes (e.g., in the GI tract). In some instances, the disease is hepatitis, obesity, fatty liver disease, liver inflammation, or pancreatitis, Crohn's disease (e.g., fistulizing Crohn's disease), ulcerative colitis (e.g., mild-to-moderate or moderate-to-severe), pouchitis, proctitis, multiple sclerosis, systemic lupus erythematosus, graft versus host disease, rheumatoid arthritis, or psoriasis.

### Purification Methods and Compositions

The present disclosure contemplates methods for obtaining a purified non-naturally occurring fusion protein. The non-naturally occurring fusion protein can comprise IL-22 and a carrier, and optionally a spacer coupling the IL-22 to the carrier. The non-naturally occurring fusion protein can be a protein comprising, consisting essentially of, or consisting of an amino acid sequence having at least 80%, 85%. 90%, 95%, 98%, or 99% sequence identity, or having 100% sequence identity, to an amino acid sequence set forth in SEQ ID NOS: 14-21, 24, or 25. Advantages of the methods and compositions described herein include maintaining high purity and biological activity of the purified polypeptides or proteins.

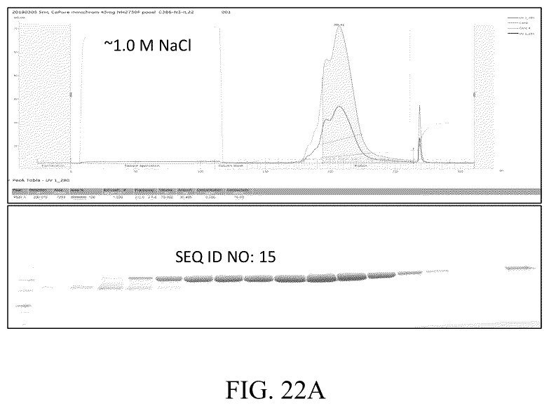

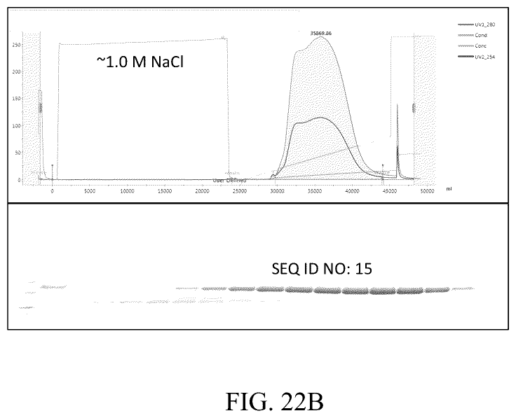

Specific advantages of the herein disclosed methods include: a) high biological activity of the non-naturally occurring fusion proteins due to correct folding using specifically developed folding buffers; b) high chemical purity of the purified non-naturally occurring fusion proteins paired with low toxicity; c) high recovery yield of the material subjected to purification; d) reproducibility of the results enables reliable production and supply; e) manufacturability and scalability to multi-gram and multi-kilogram scale for clinical and commercial applications; f) sustainable use of materials and resources provide a cost- and logistically effective purification method for clinically relevant molecules. Additionally, improved manufacturing methods can increase the speed at which these purified proteins can be developed for therapeutic use (FIG. 22).

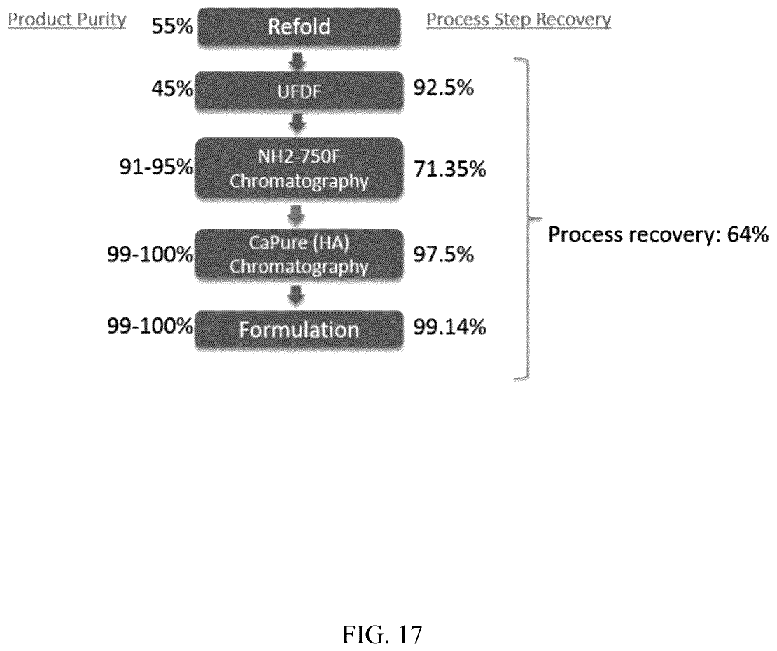

An exemplary process for purification of the non-naturally occurring fusion proteins described herein is shown in FIG. 17, which further illustrates exemplary purity and recovery amounts for the non-naturally occurring fusion protein at each stage in the purification process.

As used herein, “purity,” indicates a level of the desired protein (e.g. non-naturally occurring fusion protein) or desired form of the protein (e.g. a monomer of the non-naturally occurring fusion protein, a corrected folded non-naturally occurring fusion protein, or a combination thereof) in a composition which can also comprise non-desired proteins or non-desired forms of the protein (e.g. an aggregate of the non-naturally occurring fusion protein, an incorrectly folded protein, or a combination thereof). The level can be represented by a percentage (%) of the desired protein or desired form of the protein. For example, the purity can be greater than 40%, 50%, 60%, 70%, 80%, 90%, 95%, or 99%, or can be 100%. The term “purified” can be used to indicate a composition which has experienced an increase in purity. An increase in purity can be due to purification procedures described herein, such as anion exchange chromatography, hydroxyapatite chromatography, cation exchange chromatography, or a combination thereof.

In some cases, the methods and compositions of the present disclosure comprise the use of at least two chromatography columns that are performed in tandem, follow by a concentration/buffer exchange step (ultrafiltration/diafiltration (UF/DF) to concentrate and buffer exchange the solution). The chromatography columns can be low pressure chromatography systems, such as the AKTA Avant 150 column or an AKTA Pilot column from General Electric (GE). In some cases, at least one column contains an anion exchange resin (e.g. NH₂-750F resin) and at least one column contains a hydroxyapatite resin (e.g., CaPure® resin). In some case, at least one column containing the anion exchange resin is used for the capture step, whereas the at least one column containing the hydroxyapatite resin is used for the polishing step. In some cases, instead (or in addition to) the hydroxyapatite column, a cation exchange column may be used. For instance, in some embodiments, the cation exchange column with a sulfate-functionalized polymethacrylate resin, such as TOYOPEARL Sulfate-650F. The purity of the solubilized protein can increase from 44% to 47% following UF/DF to from 93% to 97% following anion exchange chromatography to at least 99% following hydroxyapatite chromatography. The recovery of the solubilized protein can increase from 70% to 73% following the anion exchange chromatography to at least 97% following the hydroxyapatite chromatography.

### Isolation of Non-Naturally Occurring Fusion Proteins From Inclusion Bodies

In some embodiments, the non-naturally occurring fusion proteins to be purified are isolated from inclusion bodies (IBs). IBs as described herein may be nuclear or cytoplasmic aggregates of stable substances, such as proteins and polypeptides. In some embodiments, double-washed inclusion bodies (DWIB) comprising the non-naturally occurring fusion proteins (e.g., SEQ ID NOs: 14-21) from fermentation is resuspended in a specific buffer system (see EXAMPLE 6).

In some cases, the concentration of DWIB is from about 0.5 g DWIB/100 mL buffer to about 5 g DWIB/10 mL buffer. In some cases, the concentration of DWIB is from about 2 g DWIB/50 ml buffer to about 5 g DWIB/50 mL buffer. In some cases, the concentration of DWIB is from about 1 g DWIB/20 mL buffer to about 1 g DWIB/10 mL buffer. In some cases, the concentration of DWIB is at least 1 g DWIB/100 mL buffer. In some cases, the concentration of DWIB is at least 1 g DWIB/10 mL buffer.

The buffer system for resuspension may comprise a variety of buffer agents and ingredients. In some cases, the buffer system for resuspension comprises Guanidine/HCl (Gu-HCl) at a concentration of at least 1M, at least 2 M, at least 4M, at least 6 M, at least 8M. In some cases, the concentration of Gu-HCl is from about 4 M to about 8 M. In some cases, the buffer system for resuspension comprises Tris buffer, wherein the concentration of Tris is at least 5 mM, at least 10 mM, at least 20 mM, at least 50 mM, or at least 100 mM. In some cases, the Tris buffer has a concentration from about 40 mM to about 60 mM. The Tris buffer can be Tris-HCl. The Tris-HCl can have a pH from 8.0 to 8.5. The Tris-HCl can have a pH of 8.2. In some cases, the buffer system for resuspension comprises dithiothreitol (DTT). The DTT can have a concentration of at least 7 mM, 8 mM, 9 mM, 10 mM, 20 mM, 30 mM, 40 mM, 50 mM, 60 mM , or 70 mM. The DDT can have a concentration from 7 mM to 11 mM, from 8 mM to 10 mM, or from 9 mM to 10 mM. In some cases, the buffer system for resuspension comprises ethylenediaminetetraacetic acid (EDTA). The EDTA can have a concentration of at least 1 mM, 1.5 mM, 2 mM, or 2.5 mM. The EDTA can have a concentration from 1 mM to 2.5 mM or from 1.9 mM to 2.1 mM. In some embodiments, the buffer system for resuspension comprises, consists essentially of, or consists of a Tris buffer, Gu-HCl, and DTT. The buffer system used for resuspension may have a pH from about 6 to about 10. In some cases, the buffer system has a pH from about 7.5 to about 8.5. In some cases, the pH of the buffer system is at least 7. In some cases, the pH of the buffer system is at least 8. In some cases, the pH of the buffer system is at least 8.5.

### Reduction of Solubilized Non-Naturally Occurring Fusion Proteins

A reducing agent may be added to the resuspension solution. The reducing agent may be dithiothreitol (DTT). The amount of reducing agent used may depend on the volume of the resuspension solution and the fusion protein present in that solution. In some cases, the amount of reducing agent used is from about 0.025 mM to about 50 mM, from 0.5 mM to 30 mM, from 1mM to 10 mM. In some cases, the amount of reducing agent used is at least (or between) 2 mM, 3 mM, 4 mM, 5 mM, 6 mM, 7 mM, 8 mM, 9 mM, or 10 mM.

The solubilized non-naturally occurring fusion protein (e.g., SEQ ID NOs: 14-21) solution may be incubated at various temperatures ranging from about 2° C. to about 40° C. In some cases, the protein solution may be incubated at room temperature (RT). Incubation may be performed while the solution is stirring. The solution may be stirred on a magnetic stir-plate and then centrifuged. Centrifugation may be performed at about 1,000×g to about 20,000×g. In some cases, centrifugation is performed at 15,970×g for 90 minutes at 4° C. The incubation time may vary from about 20 minutes to about 180 minutes, depending on the concentration of ingredients, the concentration of the fusion protein (e.g., SEQ ID NOs: 14-21).

Protein concentration in various solutions throughout the methods and procedures as described herein may be performed using Bradford assay. Concentration of the solubilized non-naturally occurring fusion protein may be from about 1 mg/mL to about 50 mg/mL. In some cases, the final concentration of the solubilized non-naturally occurring fusion protein may be from about 3 mg/mL to about 30 mg/mL. In some cases, the final concentration of the solubilized non-naturally occurring fusion protein may be from about 5 mg/mL to about 20 mg/mL. In some cases, the final concentration of the solubilized non-naturally occurring fusion protein may be at least 15 mg/mL.

### Refolding of Non-Naturally Occurring Fusion Proteins

The non-naturally occurring fusion proteins of the present disclosure may exert their biological activity due to their specific three-dimensional structure or folding. Therefore, refolding of these polypeptides can be important in developing these polypeptides for pharmaceutical applications. Desirable refolding of a fusion protein comprising at least a carrier and an IL-22 can comprise refolding of the carrier or the IL-22 into a tertiary structure similar to a tertiary structure of a homologous naturally occurring carrier or IL-22 sequence or a tertiary structure which results in maintenance of the desired activity of the carrier (e.g. transcytosis of the fusion protein) and IL-22. The methods described herein can comprise refolding the solubilized non-naturally occurring fusion protein to produce refolded non-naturally occurring fusion protein. The refolding can occur prior to performing the anion exchange chromatography.

The solubilized protein (e.g., SEQ ID NOs: 14-21) may be added to a refolding solution, also referred to as a refold buffer solution. The amount of solubilized protein used may be from 0.1 mg/mL to 1 mg/mL, 1 mg/mL to 100 mg/mL, from 1 mg/mL to 50 mg/mL, from 1 mg/mL to 10 mg/mL, from 5 mg/mL to 20 mg/mL, or about 15 mg/mL. The amount of solubilized protein can be at least 1, 2, 3, 4, 5, 6, 7, 8, 9, or 10 mg/mL. The amount of solubilized protein can be no more than 0.1, 0.5, 1, 5, 10, 20, 30, 40, 50, 60, 70, 80, 90, or 100 mg/mL. The refolding solution can have a pH of between 7.5 and 8.5, or about 7.0.

The refold buffer solution can comprise an amino acid, a polyol, a salt, a sugar, a redox reagent, a chaotrope, or a combination thereof. The amino acid can be proline, glycine, arginine, or alanine. The polyol can be glycerol. The salt can be sodium chloride (NaCl), potassium chloride (KCl), or magnesium chloride (MgCl₂). The sugar can be glucose or sucrose. The redox reagent can be cysteine, cystamine, glutathione, dithiothreitol (DTT), or copper sulfate (CuSO₄). In some embodiments, the refold buffer comprises Tris-base, Arginine-HCl, urea, EDTA, glycerol, L-Cysteine, Cystamine-2HCl, DTT, Gu-HCl, or a combination thereof. In some embodiments, the refold buffer comprises, consists essentially of, or consists of Tris pH 8.5, arginine glycerol, cysteine, and cystamine. Concentrations of Tris buffer may range from about 20 mM to about 200 mM with pH ranging from about 6 to about 9. Concentrations of Arginine-HCl may range from about 0.1 M to about 1.5 M, from about 0.75 M to 1.25M, or about 1.0 M. Concentrations of urea may range from about 0.5 M to 1.5 M. Concentrations of EDTA may range from 1 mM to 3 mM. Concentrations of glycerol may range from about 2% v/v to about 20% v/v, from about 8% v/v to about 12% v/v, or about 10% v/v. Concentrations of cysteine can be from 1 mM to 5 mM, from 2 mM to 4 mM, or about 3 mM. The cysteine can be L-cysteine. Concentrations of cystamine may range from about 0.5 mM to about 5 mM, from about 1 mM to about 4 mM, or about 3 mM. The cystamine can be cystamine-2HCl. Concentrations of DTT may range from about 0.1 mM to about 1 mM. Concentrations of Gu-HCl may range from about 50 mM to about 500 mM. The refold buffer can comprise, consist essentially of, or consist of Tris, L-arginine, urea, EDTA, cysteine, and cystamine. The refold buffer can comprise, consist essentially of, or consist of 100 mM Tris pH 8.5, 1M arginine, 10% (v/v) glycerol, 3 mM L-Cysteine, and 1 mM Cystamine-2HCl. The solubilized non-naturally occurring fusion protein can be added to the refold buffer to produce a refold mixture. The concentration of the non-naturally occurring fusion protein, after addition to the refold mixture, can be from about 0.1 mg/mL to 1.0 mg/mL, 0.5/mL mg to 1.5 mg/mL, from 0.75 mg/mL to 1.25 mg/mL, or about 1 mg/mL. The concentration of the non-naturally occurring fusion protein in the refold mixture can be less than 0.5 mg/mL, 0.6 mg/mL, 0.7 mg/mL, 0.8 mg/mL, 0.9 mg/mL or 1.0 mg/mL.

In various embodiments, a volume of about 100 mL to about 10,000 L refold buffer is used. In some cases, the volume is from about 50 mL to about 1500 L. In some cases, the volume is from about 10 L to about 300 L. In some cases, the volume is at least 200 L.

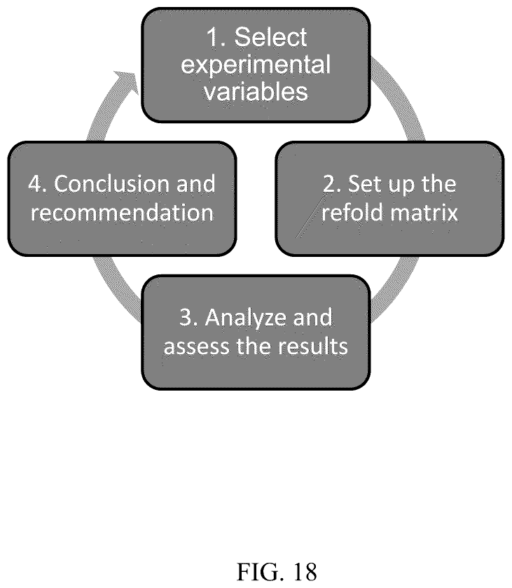

The refold solution may have a certain temperature. For example, the refold solution may be pre-chilled to about 2-12° C. In some cases, the refold solution is pre-chilled to at least about 3° C. This refolding process may be optimized depending on the protein used and the application (FIG. 18).

In some embodiments of the present disclosure, the refolding mixture is incubated for a certain period of time. In some cases, the process may be about one hour, two hours, four hours, five hours, or 20 hours. In some cases, the refolding process takes about 15 to about 25 hours. In some cases, a peristaltic pump set at a certain flow rate depending on the constitution of the solution (e.g., concentration or volume) to deliver the solubilized material into the refold solution. In some cases, a peristaltic pump set at a flow rate of 60-80 ml/min. The optimization process utilized a design of experiments (DOE) of 15 different matrices, 12 variables and 200 refold reactions. Decisions were made by a process of elimination.

In various embodiments, quantitative analysis may be performed to determine the amount or percentage of correctly folded protein in the solution comprising the refolded non-naturally occurring fusion proteins. In some instances, SEC-HPLC is performed to determine the amount of the properly folded non-naturally occurring fusion protein present in the refold samples.

The purity of the refolded non-naturally occurring fusion protein can be from 45% to 65%, from 40% to 60%, or from 45% to 55%. The purity of the refolded non-naturally occurring fusion protein can be at least 40%, 45%, 50%, or 55%.

### Tangential Flow Filtration (TFF) of Solubilized IL-22 Delivery Constructs

In various embodiments, protein refolding is performed in combination with or prior to tangential flow filtration (TFF) systems (e.g., Millipore) and ultrafiltration/diafiltration (UF/DF) systems with certain molecular weight cut-offs (MWCOs).

Protein refolding mixture may be subsequently processed by TFF to concentrate and buffer exchange the solution. In some cases, the process is performed by ultrafiltration/diafiltration (UF/DF). During ultrafiltration, the solution can be concentrated from 8-fold to 12-fold, or about 10-fold. Ultrafiltration can be followed by a 5-fold buffer exchange during diafiltration with a diafiltration buffer. The UF/DF can comprise the use of 10-20 kDa MWCO Millipore Ultracell Pellican3 filters, with 1-2 m2 TFF flat sheet cassettes. The specific parameters (i.e. TMP) may be varied depending on the molecular characteristics of the protein. The diafiltration buffer can comprise from 10-25 mM Tris-base at pH from about 7 to about 8.5, and from 50-200 mM NaCl. The specific parameters may be varied depending on the molecular characteristics of the protein. The final volume at the end of the UF/DF process may be from about 5 L to about 100 L. In some cases, the final volume ranges from about 10 to about 50 L. In some cases, the final volume ranges from about 80 mL to about 10 L.

Subsequent filtration may be carried out using a flow filtration system. In some cases, the flow filtration system is an AkroPac® filter system and Cole-Parmer® peristaltic pump and tubing. Bradford assay may be performed to determine the protein concentration, yield, and step recovery.

In various embodiments, quantitative analysis may be performed to determine the amount or percentage of correctly folded protein in the sample solutions. In some instances, SEC-HPLC is performed to determine the amount of the properly folded non-naturally occurring fusion protein present in the refold and UF/DF samples.

The purity of a solution comprising the refolded non-naturally occurring fusion protein following UF/DF can be from 35% to 55%, from 40% to 50%, from 43% to 47%, or about 45%. The purity of a solution comprising the refolded non-naturally occurring fusion protein following UF/DF can be at least 35%, 40%, or 45%. The recovery of the solubilized, refolded non-naturally occurring fusion protein following UF/DF can be from 87% to 97%, from 90% to 94%, from 92% to 93%, or about 92.5%. (Other concentration and buffer exchange systems can also be used.)

### Purification and Recovery of Non-Naturally Occurring Fusion Proteins

As previously described, the use of two chromatography methods in tandem, such as an anion exchange chromatography and a hydroxyapatite chromatography (or alternatively cation exchange chromatography), can result in increased purity and recovery of the non-naturally occurring fusion protein, such as the IL-22 delivery constructs described herein, from solution. In various embodiments of the present disclosure, the chromatography specifics (e.g., column type, resin, flow rate, buffer systems, gradient, and conductivity) are determined and optimized depending on various parameters, e.g., the protein to be purified. A variety of purification columns may be used for protein purification as described herein.

### Anion Exchange Chromatography

The methods described herein can comprise performing anion exchange chromatography (AEX) on a mixture comprising the non-naturally occurring fusion protein. The mixture can be a solution comprising the refolded non-naturally fusion protein. Performing anion exchange chromatography can produce a first fraction comprising the non-naturally occurring fusion protein.

A variety of resins may be used in combination with the methods and compositions of the present disclosure. In some cases, the resin comprises amine-functionalized polymethylacrylate beads. In some cases, anion exchange resin NH₂-750F is used for protein purification. A column with a bed-height of at least 15 cm, 20 cm, 25 cm, or 30 cm can be filled with the resin. A column with a bed-height from 10 to 50 cm may be filled with resin in a way to ensure a column volume that may facilitate an appropriate dynamic binding capacity (e.g., >20 g/L) to enable production of the desired quantity of protein. In some cases, the dynamic binding capacity of the column is from 5 g/L to 100 g/L, from 20 g/L to about 50 g/L, from 15 g/L to 30 g/L, or from 20 g/L to 25 g/L.

Buffer systems used for elution (e.g., gradient elution) in anion exchange chromatography may comprise one, two, three, or four different buffer solutions (e.g., Buffers A through D). In some embodiments, two buffers are used in the anion exchange chromatography and are referred to herein as Buffer A and Buffer B. In some cases, a buffer solution comprises Tris (e.g., 10-50 mM, pH 7-9), and/or NaCl (e.g., 0.1-5 M). In some cases, the buffer solution further comprises glycerol. In some embodiments, the buffer solution(s) used in anion exchange chromatography, such as for example Buffer A or Buffer B, comprises, consists essentially of, or consists of Tris pH 7.5 and NaCl, and optionally, glycerol.

Buffer A can comprise from 10 mM to 30 mM, from 15 mM to 25 mM, from 19 mM to 21 mM, or about 20 mM Tris pH 7.5. Buffer A can comprise from 0.25 M to 0.95 M, from 0.35 M to 0.75M, from 0.45 M to 0.55 M, about 0.5 M, from 0.65 M to 0.75 M, or about 0.7 M NaCl. Buffer B can comprise from 10 mM to 30 mM, from 15 mM to 25 mM, from 19 mM to 21 mM, or about 20 mM Tris pH 7.5. Buffer B can comprise from 1 M to 3 M, from 1.5 M to 2.5 M, from 1.9 M to 2.1 M, or about 2 M NaCl. Buffer A can further comprise from 8% to 12%, from 9% to 11%, or about 10% (v/v) glycerol. Buffer B can further comprise from 8% to 12%, from 9% to 11%, or about 10% (v/v) glycerol.

For anion exchange chromatography of SEQ ID NO: 15, Buffer A can comprise 20 mM Tris pH 7.5 and 0.5 M NaCl and Buffer B can comprise 20 mM Tris pH 7.5 and 2.0 M NaCl (TABLE 4). For anion exchange chromatography of SEQ ID NO: 17, Buffer A can comprise 20 mM Tris pH 7.5 and 0.5 M NaCl and Buffer B can comprise 20 mM Tris pH 7.5 and 2.0 M NaCl. For anion exchange chromatography of SEQ ID NO: 14, Buffer A can comprise 20 mM Tris pH 7.5, 0.7 M NaCl, and 10% (v/v) glycerol, and Buffer B can comprise 20 mM Tris pH 7.5, 2.0 M NaCl, and 10% (v/v) glycerol (TABLE 5).

In some embodiments, a certain volume of a salt solution is added to the protein solution. In some cases, NaCl solution with a concentration raging from about 0.1 to about 5 M is added to the protein solution.

Protein solutions may be loaded onto the column such that a specific flow-rate and a specific column residence time may be observed to ensure appropriate interaction of the protein and the solid phase (e.g., resin). In some cases, the flow rate is controlled such that a column residence time is from about 30 seconds to about 6 minutes, or about 5 minutes. The column residence time can be at least 30 seconds, or 1, 2, 3, 4, or 5 minutes. The column residence time can be no more than 3, 4, 5, 6, 7, 8, 9, or 10 minutes. The flow-through may be collected and analyzed to determine if there is unbound protein, which is considered as a loss. For protein analysis and purification, the absorbance at about 280 nm is determined for protein concentration and SEC-HPLC for protein purity.

In some embodiments, a percentage (e.g., 0-100%) or flow rate (e.g., 1-10 mL/min) of a first buffer is combined with a second buffer to achieve a specific final flow rate (e.g., 1-10 mL/min) of the column purification system. For example, a linear gradient of 20 column volumes (CV) from 0.0-62.5% B (100-37.5% A), followed by a step gradient to 100% B (0% A) for additional 5 CV is performed for protein purification. In some cases, a gradient of 0-75% B (100-25% A) 40 CV, Step 100% B, 10 CV may be performed as an elution gradient indicating 91-93% pure protein and a recovery of >90%.

The purity of the non-naturally occurring fusion protein in the first fraction can be from 90% to 99% or from 91% to 95%. The purity of the solubilized non-naturally occurring fusion protein in the first fraction can be at least 90%, 91%, 92%, 93%, 94%, or 95%. The recovery of the solubilized non-naturally occurring fusion protein in the first fraction can be from 61% to 81%, from 66% to 76%, or from 71% to 72%.

### Cation Exchange Chromatography or Hydroxyapatite Chromatography

The methods described herein can further comprise subjecting the first fraction obtained following the anion exchange chromatography to a cation exchange resin to obtain a second fraction comprising the non-naturally occurring fusion protein. The cation exchange resin can be a sulfate-functionalized methacrylate resin. The cation exchange resin can be a TOYOPEARL® Sulfate-650F resin.

The methods described herein can additionally or alternatively comprise subjecting the first fraction obtained following the anion exchange chromatography to a hydroxyapatite resin to obtain a second fraction comprising the non-naturally occurring fusion protein. The hydroxyapatite resin can be a cation exchange resin further comprising a calcium affinity. The hydroxyapaptite resin can comprise calcium phosphate. The hydroxyapaptite resin can comprise a chemical formula of: Ca₁₀(PO₄)₆(OH)₂. The hydroxyapaptite resin can comprise a particle size from 30 μm to 50 μm, from 35 μm to 45 μm, or about 39 μm. The hydroxyapaptite resin can be the CaPure® resin.

Advantages of the CaPure® resin can include a) salt tolerance, allowing protein adsorption at high conductivity in aqueous solutions; b) no preparation of proteins (e.g., fusion proteins such as SEQ ID NO: 14-21) required prior to load; c) results in a high recovery (>90%); d) high binding capacity (>20 mg/mL resin); e) increases purity by removal of low molecular weight (LMW) impurities; and f) ensures endotoxin clearance (<1.0 EU/mg).

The cation exchange resin or hydroxyapatite resin can be used to pack a chromatograph column. The column can comprise a bed height from 10 cm to 50 cm, or about 20 cm. The column can comprise a bed height of at least 10 cm, 15 cm, 20 cm, 25 cm, or 30 cm. The column can comprise a bed height of no more than 20 cm, 30 cm, 40 cm, or 50 cm. In some cases, the hydroxyapatite mixed-mode resin CaPure® is used to pack a column with a bed-height of 10-50 cm, ensuring a column volume that facilitates a dynamic binding capacity of maximum 10-100 g/L to enable production of the desired quantity of protein.

Buffer systems used for elution (e.g., gradient elution) in hydroxyapatite chromatography may comprise one, two, three, or four different buffer solutions (e.g., Buffers A through D). In some embodiments, two buffers are used in the hydroxyapatite chromatography and are referred to herein as Buffer A and Buffer B. In some cases, a buffer solution comprises Tris (e.g., 10-50 mM, pH 7-9), and/or NaCl (e.g., 0.1-5 M). In some cases, the buffer solution further comprises glycerol. In some embodiments, a first buffer solution used in hydroxyapatite chromatography, such as for example Buffer A, comprises, consists essentially of, or consists of Tris pH 7.5, NaCl, and CaCl₂. In some embodiments, a second buffer solution used in hydroxyapatite chromatography, such as for example Buffer B, comprises, consists essentially of, or consists of sodium phosphate pH 7.0, NaCl, and CaCl₂.

Buffer A can comprise from 10 mM to 30 mM, from 15 mM to 25 mM, from 19 mM to 21 mM, or about 20 mM Tris pH 7.5. Buffer A can comprise from 80 mM to 120 mM, from 90 mM to 110 mM, or about 100 mM NaCl. Buffer A can comprise from 0.5 mM to 1.5 mM, from 0.75 mM to 1.25 mM, from 0.9 mM to 1.1 mM, or about 1 mM of CaCl₂. Buffer B can comprise from 150 mM to 250 mM, from 175 mM to 225 mM, from 190 mM to 210 mM, or about 200 mM sodium phosphate pH 7.0. Buffer B can comprise from 50 mM to 150 mM, from 75 mM to 125 mM, from 90 mM to 110 mM, or about 100 mM NaCl. Buffer B can comprise from 0.5 mM to 1.5 mM, from 0.75 mM to 1.25 mM, from 0.9 mM to 1.1 mM, or about 1 mM of CaCl₂.

For hydroxyapatite chromatography of SEQ ID NO: 15, Buffer A can comprise 20 mM Tris pH 7.5, 100 mM NaCl, and 1 mM CaCl₂ and Buffer B can comprise 200 mM sodium phosphate pH 7.0, 100 mM NaCl, and 1 mM CaCl₂ (TABLE 6). For hydroxyapatite chromatography of SEQ ID NO: 17, Buffer A can comprise 20 mM Tris pH 7.5, 100 mM NaCl, and 1 mM CaCl₂ and Buffer B can comprise 200 mM sodium phosphate pH 7.0, 100 mM NaCl, and 1 mM CaCl₂. For hydroxyapatite chromatography of SEQ ID NO: 14, Buffer A can comprise 20 mM Tris pH 7.5, 100 mM NaCl, and 1 mM CaCl₂ and Buffer B can comprise 200 mM sodium phosphate pH 7.0, 100 mM NaCl, and 1 mM CaCl₂ (TABLE 7).

The purity of the solubilized protein following the use of cation exchange resin or hydroxyapatite resin can be from 99% to 100%. The purity of the solubilized protein following the use of hydroxyapatite resin can be at least 95%, 96%, 97%, 98%, 99%, or 100%. The recovery of the solubilized protein following the use of hydroxyapatite resin can be from 85% to 100%, from 96% to 99%, or from 97% to 98%. The hydroxyapatite resin can be CaPure®.

Fractions collected during column chromatography that contain the purified compound (e.g., fusion protein) may be concentrated and formulated for administration using buffer exchange or diafiltration. For example, fractions collected during CaPure® can be concentrated and formulated using a TFF system (e.g., Pall corporation) to concentrate the protein. The protein can be concentrated to a final concentration of 20 mg/mL followed by 5-fold buffer exchange. The filtration process may be performed using ultrafiltration/diafiltration (UF/DF) and filters with a 10 kDa MWCO Millipore Pellican3, 0.114 m² TFF flat sheet cassettes. The MWCO of the filtration system may be varied depending on the protein (e.g., molecular weight) to be purified. The diafiltration buffer may consist of 10-20 mM sodium phosphate at about pH 7.0, 50-100 mM NaCl. Formulated SEQ ID NOS: 14-21 may subsequently be filtered using a flow filtration system employing an AkroPac® 0.8/0.2 um filter and a Cole-Parmer® peristaltic pump and tubing. Purified and formulated protein may be stored in aliquots at −80° C. until further use.

In specific embodiments, proteins analyzed by SEC-HPLC may show above 97% purity (typically >98%) using TSKgel GW3000SWXL, 5 μm, 7.8 mm ID×30.0 cm L column (Tosoh Bioscience, 8541).

### Protein Analysis

Samples from different fractions may be analyzed by SDS-PAGE using the Bio-Rad ChemiDoc™ MP imaging system, and the fractions collected during column chromatography may be analyzed for protein content using Thermo Fisher Nanodrop One™. General techniques for protein detection or analysis include gel electrophoresis, fluorescence microscopy, capillary electrophoresis, mass spectrometry, electrophoretic mobility-shift assay, or nuclear magnetic resonance.

### Methods of Treatment

In various embodiments of the present disclosure, pharmaceutical compositions comprising the fusion molecules of the disclosure are provided for use in treating and/or preventing inflammatory diseases. These pharmaceutical compositions can be formulated for oral delivery. “Inflammatory diseases” may include all diseases associated with acute or chronic inflammation. Acute inflammation is the initial response of the body to harmful stimuli and results from an increased movement of plasma and leukocytes (such as e.g. granulocytes) from the blood into the injured tissues. A number of biochemical events propagates and matures the inflammatory response, involving the local vascular system, the immune system, and various cells within the injured tissue. Prolonged inflammation is referred to as chronic inflammation, which leads to a progressive shift in the type of cells present at the site of inflammation and is characterized by simultaneous destruction and healing of the tissue from the inflammatory process. Inflammatory diseases can be caused by e.g. burns, chemical irritants, frostbite, toxins, infections by pathogens, physical injury, immune reactions due to hypersensitivity, ionizing radiation, or foreign bodies, such as e.g. splinters, dirt and debris. In some embodiments, the inflammatory disease is epithelial cell injury, hepatitis, obesity, fatty liver disease, liver inflammation, pancreatitis, Crohn's disease, fistulizing Crohn's disease, ulcerative colitis, mild-to-moderate ulcerative colitis, moderate-to-severe ulcerative colitis, pouchitis, proctitis, multiple sclerosis, systemic lupus erythematosus, graft versus host disease, rheumatoid arthritis, or psoriasis.

Further described herein are methods for treating a disease or condition in a subject, comprising administering to the subject the non-naturally occurring fusion protein. In some embodiments, the disease or condition is epithelial cell injury, hepatitis, obesity, fatty liver disease, liver inflammation, pancreatitis, Crohn's disease, fistulizing Crohn's disease, ulcerative colitis, mild-to-moderate ulcerative colitis, moderate-to-severe ulcerative colitis, pouchitis, proctitis, multiple sclerosis, systemic lupus erythematosus, graft versus host disease, rheumatoid arthritis, or psoriasis. The non-naturally occurring fusion protein can be oral administered to the subject. The subject can be human.

### EXAMPLES

These examples are provided for illustrative purposes only and not to limit the scope of the claims provided herein.

### Example 1

### Expression of a Delivery Construct

In this example, the preparation of a delivery construct as a single amino acid sequence comprising a carrier sequence derived from Cholix, a spacer sequence, and a therapeutic payload, is generally described.

First, a nucleic acid sequence encoding a delivery construct of SEQ ID NO: 15 was amplified by PCR, incorporating restriction enzymes pairs of NdeI and EcoRI, PstI and PstI, AgeI and EcoRI, or PstI and EcoRI sites at two ends of the PCR products. After restriction enzyme digestion, the PCR products were cloned into an appropriate plasmid for cellular expression, which was digested with the corresponding restriction enzyme pairs. The plasmid encoded a delivery construct comprising the amino acid sequence set forth in SEQ ID NO: 15.

The delivery construct was expressed as follows: E. coli BL21(DE3) pLysS competent cells (Novagen, Madison, Wis.) were transformed using a standard heat-shock method in the presence of the appropriate plasmid to generate delivery construct expression cells, selected on ampicillin-containing media, and isolated and grown in Luria-Bertani broth (Difco; Becton Dickinson, Franklin Lakes, N.J.) with antibiotic, then induced for protein expression by the addition of 1 mM isopropyl-D-thiogalactopyranoside (IPTG) at OD 0.6. Two hours following IPTG induction, cells were harvested by centrifugation at 5,000 rpm for 10 min. Inclusion bodies were isolated following cell lysis and washed twice with water at a ratio of 1 g/10 mL. The pellet from the cell lysis was resuspended with water followed by one hour centrifugation at 10,000 rpm at 4° C. This wash was then repeated once. Double-washed IB (DWIB) therein were solubilized in a buffer containing 50 mM Tris-HCl (pH 8.2), 6 M guanidine HCl, and 10 mM dithiothreitol (DTT). Solubilized material was then diluted into a refold buffer containing 0.1 M Tris (pH=8.5 at 4° C.), 1.0 M L-arginine, 10% glycerol, 3 mM L-cysteine, 1 mM cystamine-2HCl. The refolded protein with SEQ ID NO: 15 was purified by anion exchange chromatography (Q sepharose Ion Exchange) and Superdex 200 Gel Filtration chromatography (Amersham Biosciences, Inc., Sweden). The purity of the protein was assessed by SDS-PAGE and analytic HPLC (Agilent, Inc. Palo Alto, Calif.).

The delivery construct was evaluated to verify the proper folding with regard to its anticipated molecular size. Following induction, expressed protein was collected from inclusion bodies. Using the inclusion bodies, the extent of expression of the delivery construct was verified by gel electrophoresis, and the apparent molecular weight was compared to the calculated mass.

The results demonstrated stable and efficient production of a functional delivery construct in high yield and purity.

### Example 2

### In Vitro Model Assessing Transport of Payload Across Epithelial Cell Monolayers

This example demonstrates an in vitro model designed to evaluate the transport properties of payloads or delivery constructs described herein.

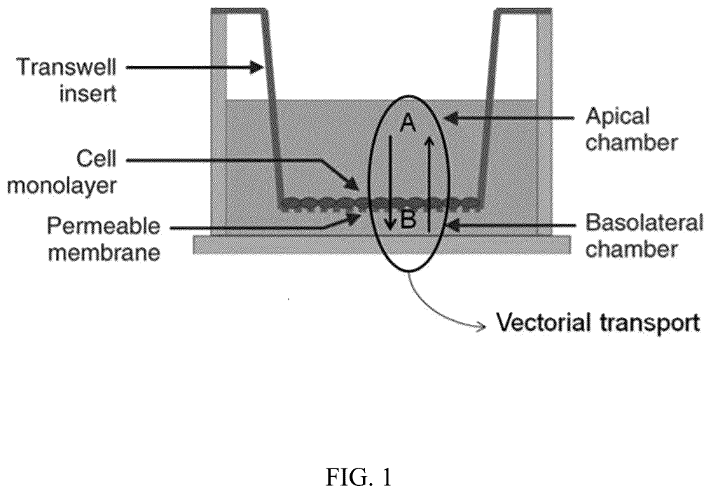

FIG. 1 schematically shows a setup comprising an apical chamber above the epithelial cell monolayer and a basal chamber below such epithelial cell monolayer.

For apical to basolateral permeability, test articles (e.g., delivery construct, payload, etc.) were added to the apical (A) side and the amount of permeation was determined on the basolateral (B) side. For basolateral to apical permeability, test articles were added to the basolateral (B) side and amount of permeation was determined on the apical (A) side.

Data can be expressed as permeability (Papp) according to the following equation: Papp=(dQ/dt)/(C0*A). Q/dt is a rate of permeation, C0 is initial concentration of test article, and A is the area of the monolayer. An efflux transport ratio (Re) can be calculated according to the following equation: (Re)=Papp(B−A)/Papp(A−B). Re>2 can indicate a potential substrate for P-gp or other active efflux transporters.

SMI-100 or Caco-2 cells can be used to assess the transcytosis function of a carrier or delivery construct in vitro.

For Caco-2 cells, an ELISA assay was performed to evaluate the ability of a carrier or delivery construct to move across Caco-2 cell monolayers via transcytosis. Caco-2 (ATCC HTB-37™) cells were maintained in 5% CO₂ at 37° C. in complete media: Dulbecco's modified Eagle's medium F12 (DMEM F12) supplemented with 10% fetal bovine serum, 2.5 mM glutamine, 100 U of penicillin/ml, and 100 μg of streptomycin/ml (Gibco BRL, Grand Island, N.Y.). Cells were fed every 2 to 3 days with this media (designated complete medium) and passaged every 5 to 7 days. For assays, cells were seeded into 24- or 96-well plates and grown to confluence.

Caco-2 cells were grown as confluent monolayers on collagen-coated 0.4-μm pore size polycarbonate membrane transwell supports (Corning-Costar, Cambridge, MA) and used 18-25 days after attaining a trans-epithelial electrical resistance (TER) of >250 Ω·cm2 as measured using a chopstick Millicell-ERS® voltmeter (Millipore). Apical to basolateral (A→B) transport of a carrier or delivery construct across these monolayer was determined by measuring the amount of transported protein at certain time points (e.g., 15, 30, and 45 minutes) after 4.7 nM, 23.6 nM and 236 nM apical application of delivery construct at 37° C. TER measurements and the extent of 10 kDa fluorescent dextran (measured using an HPLC size exclusion protocol) were used to verify monolayer barrier properties during the course of the study. The extent of transport of the delivery construct (e.g., SEQ ID NO: 15) was determined by titration of collected media in the cell-based cytotoxicity assay. Transported delivery construct was measured by enzyme linked immunosorbant assay (ELISA) using antibodies (e.g., anti-carrier or anti-payload, such as an anti-IL-22 antibody) for capture and detection.

Confluent monolayers of human small intestinal tissues (SMI-100, MatTek Corporation; Ashland, MA, USA) established on cell culture inserts were allowed to stabilize for 24 h at 37° C. prior to use. Only inserts having a trans-epithelial electric resistance (TEER) of >400 Ω·cm² were considered to have sufficient monolayer integrity for use in studies. A secondary verification of monolayer integrity was performed by assessing suppression of 70 kD dextran transport. The chambers were washed once with transport buffer (PBS). Test molecules, prepared at a concentration of 20 μg/mL, were applied to the apical surface of inserts in 100 μL volumes. Basolateral volumes of 500 μL PBS were replaced at each time point for transport studies. Each experimental condition was performed in triplicate.

### Example 3

### Carrier-Mediated Transport of IL-22 Across Polarized Gut Epithelial Cells

This example demonstrates that a carrier (SEQ ID NO: 7) can transport an IL-22 payload (SEQ ID NO: 11) across polarized gut epithelial cells in vitro. This example further demonstrates that the carrier with SEQ ID NO: 7 can transport biologically active IL-22 payload across polarized gut epithelial cells and to the lamina propria in vivo.

Transport of delivery construct (SEQ ID NO: 15) across Caco-2 cell monolayers and small intestine epithelial tissue (also referred to herein as SMI-100) was tested by applying the delivery construct to the apical membrane of the epithelial cells, according to EXAMPLE 2 and as illustrated in FIG. 1 for apical (A) to basal (B) transport. Experiments were run in duplicate and samples from the basolateral chamber were collected at 15, 30, and 45 minutes after apical application to determine the amount of transported protein. The amount of transcytosed protein was measured using ELISA assays as described in EXAMPLE 2.

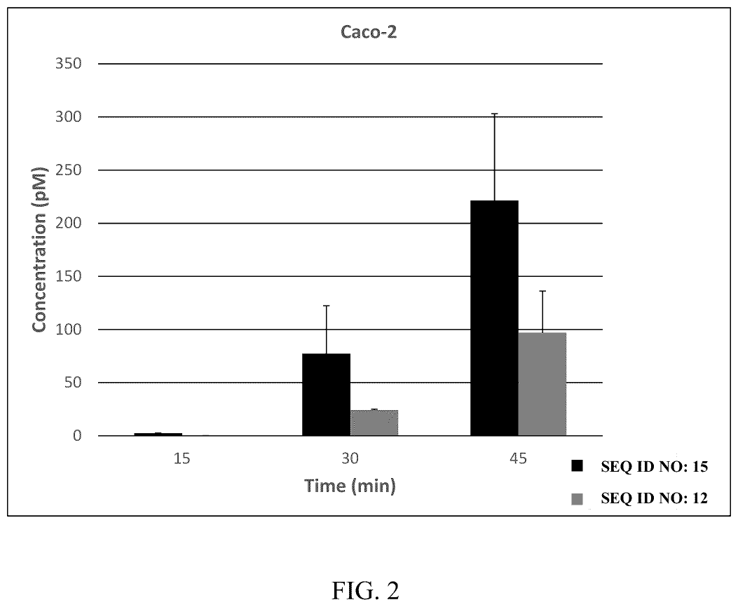

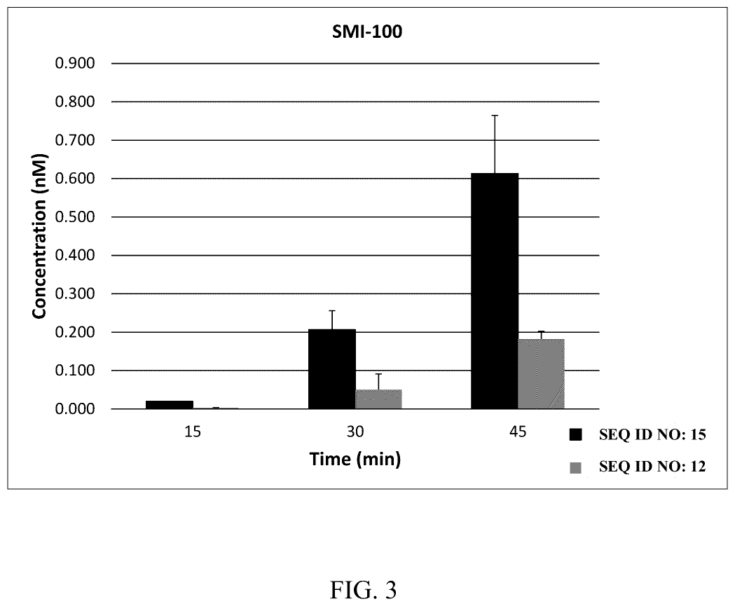

The data in FIG. 2 and FIG. 3 show that a carrier (SEQ ID NO: 7) when coupled to IL-22 (SEQ ID NO: 11) resulted in the transport of IL-22 payload across both Caco-2 (FIG. 2) and SMI-100 (FIG. 3) monolayers in a time-dependent manner, and that the delivery construct resulted in about 2-3 fold more IL-22 crossing the epithelial cells as compared to an IL-22 (SEQ ID NO: 12, M+IL-22³⁴⁻¹⁷⁹) alone that was not coupled to a carrier.

For in vivo experiments, transcytosis was tested using male Wistar rats. Male Wistar rats were housed 3-5 per cage with a 12/12 h light/dark cycle and were about 225-275 g (approximately 6-8 weeks old) when placed on study. Experiments were conducted during the light phase using a non-recovery protocol that uses continuous isoflurane anesthesia. A 4-5 cm midline abdominal incision that exposed mid-jejunum regions was conducted. Stock solutions at 3.86×10⁻⁵ M of delivery construct (SEQ ID NO: 15) were prepared in phosphate buffered saline (PBS), with 50 μL (per 250 g rat) being administered by intraluminal injection (ILI) using a 29-gauge needle. The injection site mesentery was then marked with a permanent marker. At study termination, a 3-5 mm region that captured the marked intestine segment was isolated and processed for microscopic assessment.

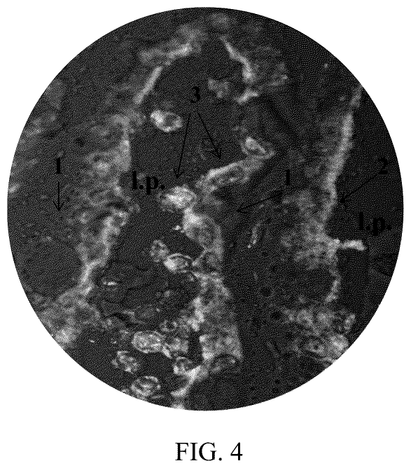

The results of the transcytosis activity of the delivery construct (SEQ ID NO: 15) are shown in FIG. 4, demonstrating that significant amounts of IL-22 payload (SEQ ID NO: 11) crossed an intact and polarized gut epithelium in vivo when provided as part of a delivery construct that includes a cholix-derived carrier (SEQ ID NO: 7) coupled to IL-22. This microscopy image shows transportation of the IL-22 payload (SEQ ID NO: 11) from the apical site of the gut epithelium (highlighted by white arrows #1) to the basal site of the epithelial cells (highlighted by white arrow #2) and into the lamina propria (abbreviated as “l.p.”) after luminal application of the delivery construct of SEQ ID NO: 15 to the jejunum of Wistar rats. The image further shows that the IL-22 interacted and bound to a significant extent to IL-22 receptors located on cells within the lamina propria and on the outer basal membrane of the polarized epithelium (highlighted by white arrows #3). IL-22 localization is indicated by white arrows #2 and #3.

### Example 4

### Recombinant Human IL-22 Binds to Murine IL-22 Receptors

This example demonstrates that recombinant human IL-22 (rhIL-22, SEQ ID NO: 12) binds to murine IL-22 receptors in a dose-dependent manner comparable to recombinant murine IL-22 (rmIL-22).

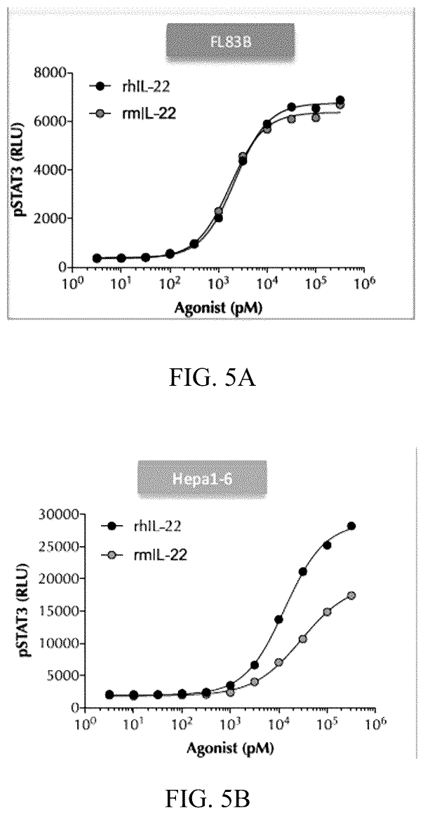

FIG. 5A demonstrates STAT3 phosphorylation in murine FL83 B cells as a function of agonist concentration (in pM), wherein the agonist is rhIL-22 (SEQ ID NO: 12) or rmIL-22. The data show that rhIL-22 and rmIL-22 induced STAT3 phosphorylation in a concentration-dependent manner, demonstrating that the human IL-22 protein can bind and induce signal transduction through mouse IL-22 receptors as effectively as mouse IL-22.

FIG. 5B demonstrates that rhIL-22 (SEQ ID NO: 12) and rmIL-22 also induced STAT3 phosphorylation in murine Hepa1-6 cells.

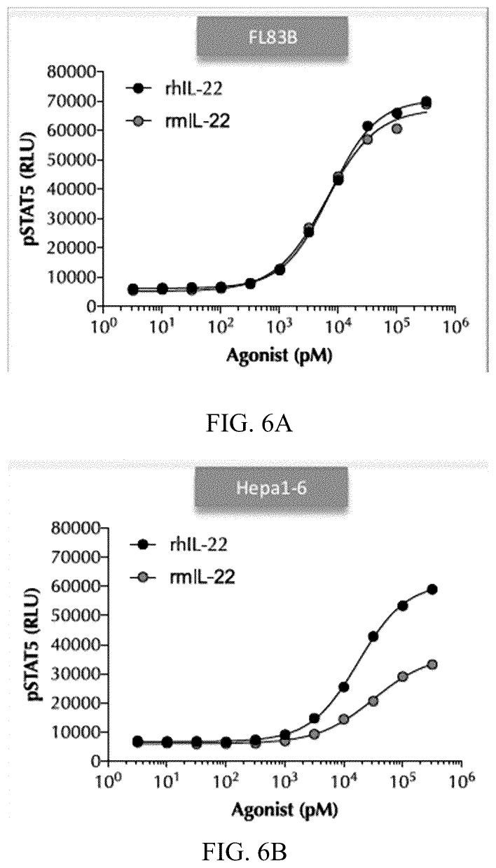

FIG. 6A demonstrates that rhIL-22 (SEQ ID NO: 12) and rmIL-22 induced STAT5 phosphorylation in murine FL83 B cells in a dose-dependent manner, demonstrating that the human IL-22 protein can bind and induce signal transduction through mouse IL-22 receptors with efficacy comparable to mouse IL-22.

FIG. 6B demonstrates that rhIL-22 (SEQ ID NO: 12) and rmIL-22 also induced STAT5 phosphorylation in murine Hepa1-6 cells but additional dose ranging will need to be performed in follow up experiments.

These data provide strong evidence that rhIL-22 (e.g., as used in the delivery constructs with SEQ ID NOs: 10 or 11) is capable of activating STAT3 and STAT5 in murine cells, thus providing rationale for using such delivery constructs in murine models to assess IL-22 activity and function.

### Example 5

### In Vivo Efficacy of the Delivery Construct With SEQ ID NO: 15 in Colitis Model

This example demonstrates the in vivo efficacy of the delivery construct with SEQ ID NO: 15 comprising the Cholix derived carrier of SEQ ID NO: 7 coupled to an IL-22 with SEQ ID NO: 11 via a linker with SEQ ID NO: 13 compared to IL-22 alone (SEQ ID NO: 12) in a dextran sulfate sodium (DSS) mouse model.

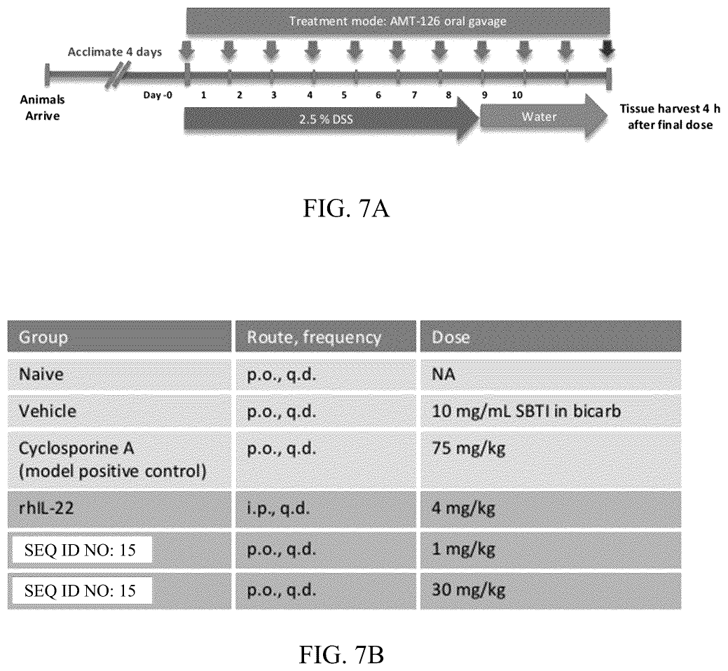

FIG. 7A shows an overview of an in vivo study design and timeline for comparing peroral (p.o.) administration of the delivery construct of SEQ ID NO: 15 at two once-daily dose levels, 1 mg/kg and 30 mg/kg, and one once daily administration (intraperitoneal (i.p.)) of rhIL-22 at 4 mg/kg. The in vivo study was performed in Normal chow-fed, 8-10 week old, ˜23 g, female C57BL/6 mice. Colitis was chemically induced with 2.5% dextran sulfate sodium (DSS) in drinking water, ad libitum. Study endpoints included percent change in body weight, disease activity index, colon length and weight, and histopathology.

FIG. 7B shows characteristics of the experimental and control groups used in the study described in FIG. 7A.

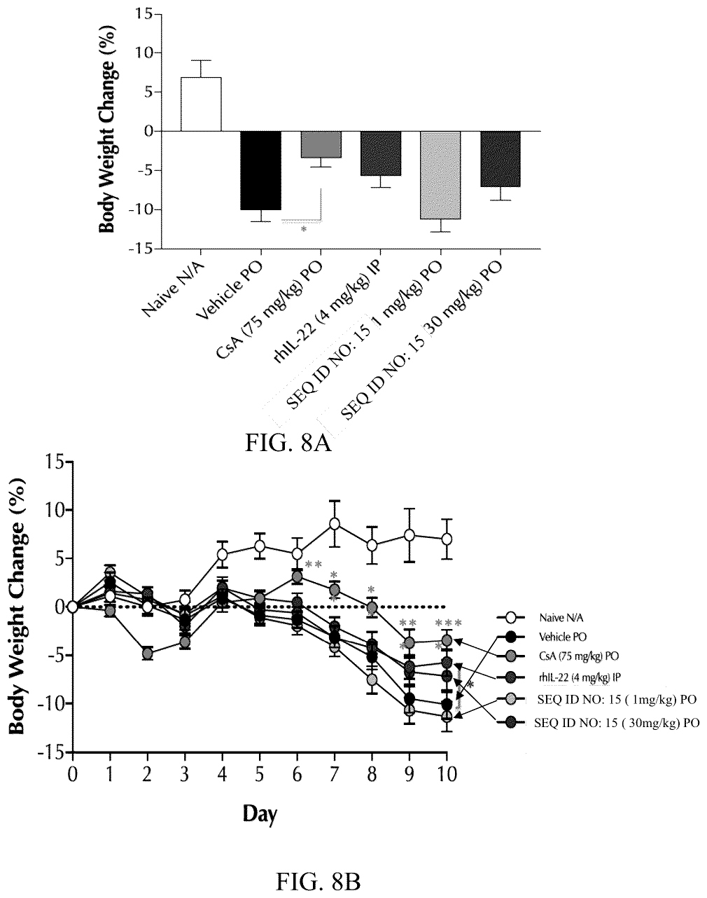

FIG. 8A shows the percent (%) change in body weight of animals at study day 10 relative to day 0 of the various experimental and control groups. Baseline (day 0 vs. 10) body weight change by rhIL-22 with SEQ ID NO: 12 or the delivery construct with SEQ ID NO: 15 relative to vehicle was not significant as assessed by 1-way ANOVA. Positive model control (cyclosporine) CsA was significantly different relative to Vehicle as assessed by a 1-way ANOVA.

FIG. 8B shows the change in body weight of experimental and control animals over the first 10 study days. CsA group mean body weight over the study period was significantly improved relative to vehicle control day 6 through day 10 as assessed by 2-way ANOVA(*, ** , ***) rhIL-22 (i.p.) body weight was significantly improved relative to vehicle control at day 10 as assessed by 2-way ANOVA (*), *p<0.05, **p<0.01, ***p<0.001.

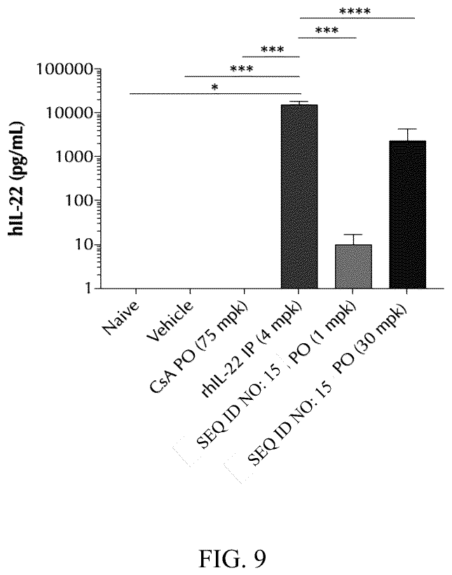

FIG. 9 shows results of plasma ELISA experiments to determine IL-22 plasma levels. The results show that plasma rhIL-22 concentrations trended towards increased levels in plasma after oral gavage of both 1 and 30 mg/kg doses of the delivery construct with SEQ ID NO: 15 in a dose-dependent manner (*p<0.05, **p<0.01, ***p<0.001. ****p<0.0001; Mean+SEM; n=2 Naïve, 5 Vehicle, 5 CsA, 10 others).

These data show that the delivery construct with SEQ ID NO: 15 induced a dose-dependent trend towards body weight improvement after administration via oral gavage. IL-22 was measured in increased levels in plasma after oral gavage of both 1 and 30 mg/kg doses of the delivery construct with SEQ ID NO: 15 in a dose-dependent manner. These data suggest that orally administered delivery construct with SEQ ID NO: 15 is capable of reducing symptoms of colitis in a DSS mouse model comparable to i.p. administered rhIL-22.

In addition, biomarkers for the orally administrable delivery construct with SEQ ID NO: 15 were evaluated by administering rhIL-22 to CD-1 mice as described below.

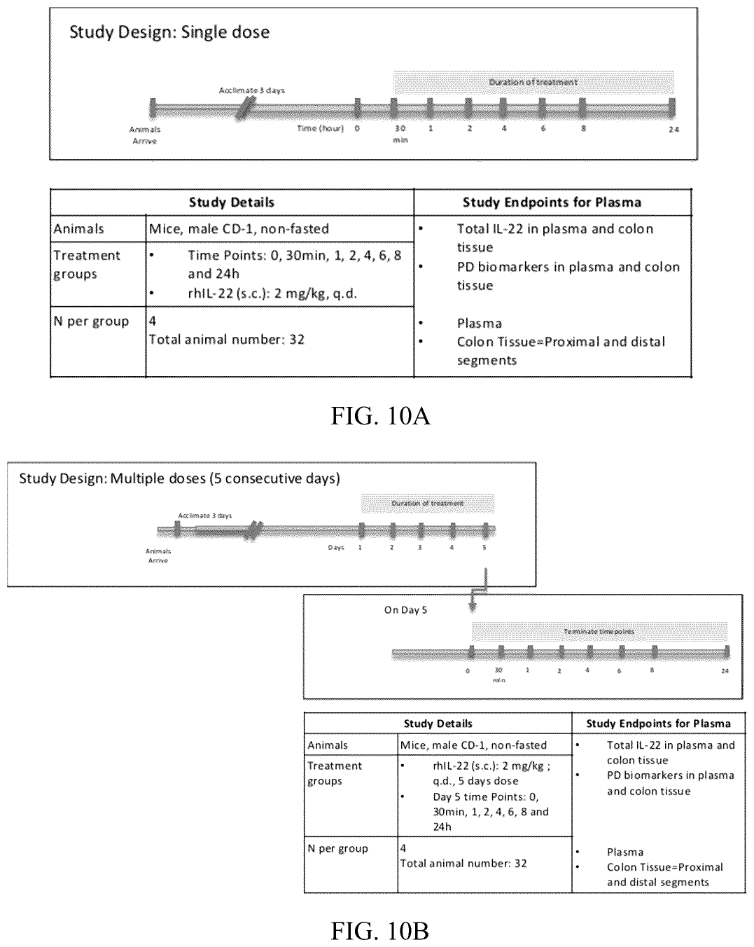

FIG. 10A shows an overview of a single dose in vivo study designed to identify biomarker(s) of target engagement after single (acute dosing) of rhIL-22 in healthy CD-1 mice.

FIG. 10B shows an overview of a multiple dose (sub-chronic dosing) in vivo study designed to identify biomarker(s) of target engagement after single and multiple doses of rhIL-22 in healthy CD-1 mice.

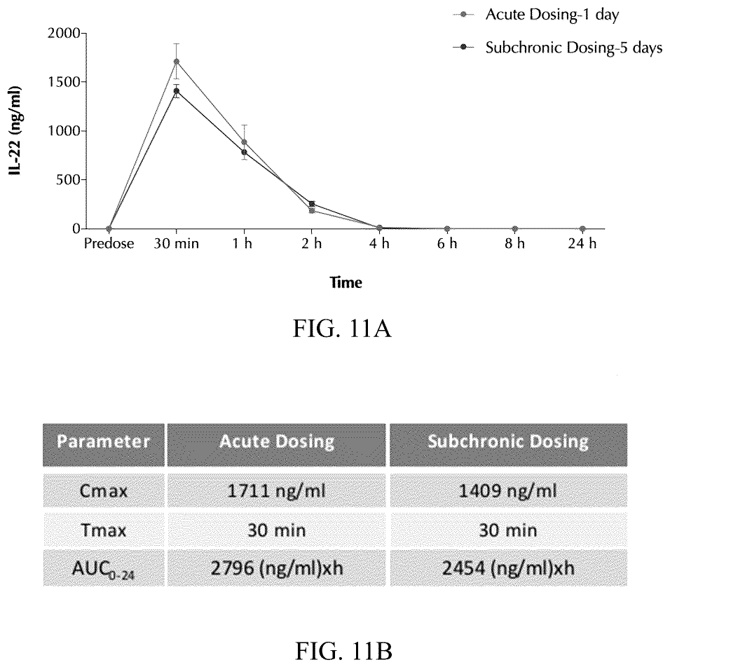

FIG. 11A shows that acute and sub-chronic administration of rhIL-22 increases IL-22 concentration consistently.

FIG. 11B shows some pharmacokinetic parameters measured during the acute and sub-chronic dosing experiments described in FIG. 11A.

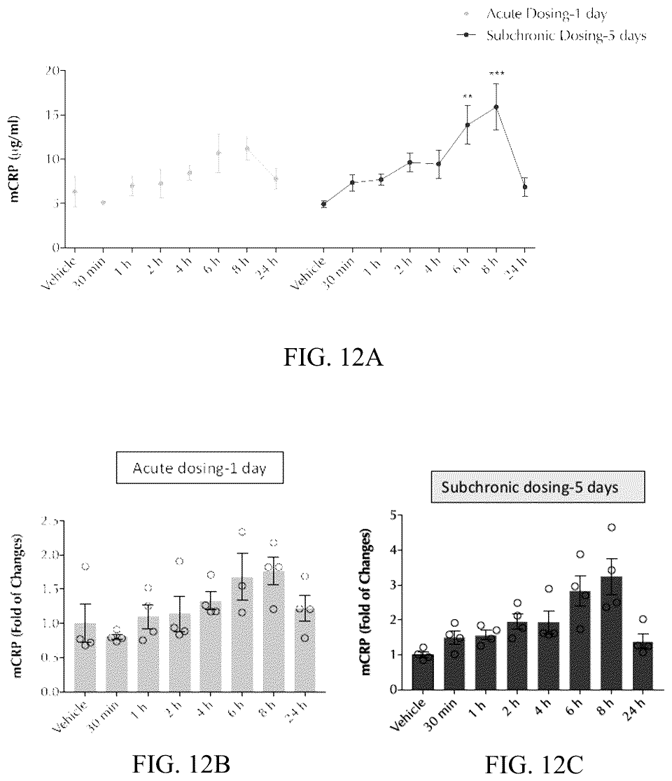

FIG. 12A shows the induced circulating murine C-reactive protein (mCRP) concentration in response to 1 day after acute dosing of rhIL-22 (SEQ ID NO: 12) and after 5 days of sub-chronic dosing of rhIL-22 (SEQ ID NO: 12). The results show a time-dependent increase in circulating mCRP in both study groups.

FIG. 12B shows fold change of plasma mCRP concentration 1 day after acute dosing of rhIL-22 (SEQ ID NO: 12).

FIG. 12C shows fold change of plasma mCRP concentration after 5 days of sub-chronic dosing of rhIL-22 (SEQ ID NO: 12).

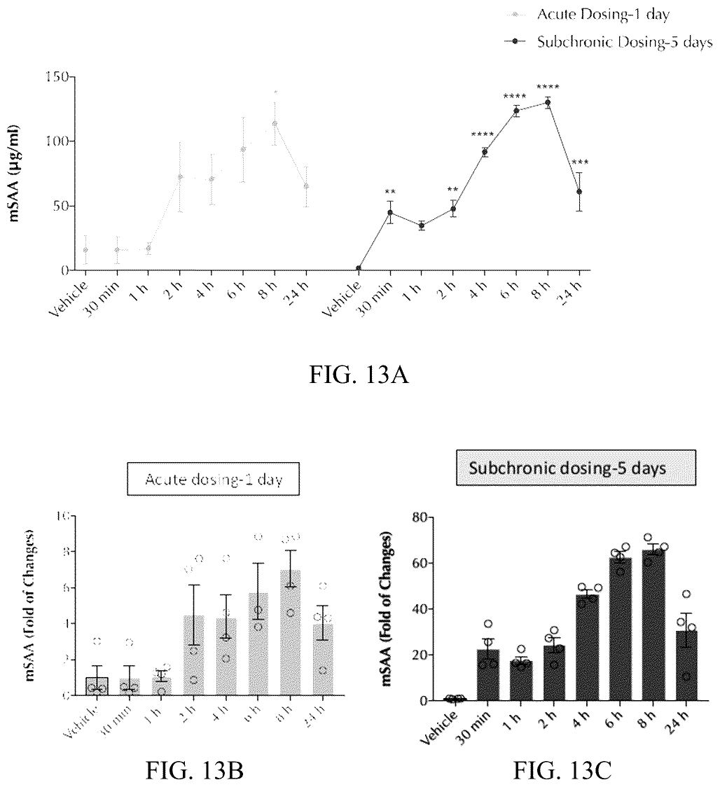

FIG. 13A shows the induced circulating murine serum amyloid protein A (mSAA) concentration in response to 1 day after acute dosing of rhIL-22 (SEQ ID NO: 12) and after 5 days of sub-chronic dosing of rhIL-22 (SEQ ID NO: 12). The results show a time-dependent increase in circulating mSAA in both study groups, with approximate plasma peak concentrations about 8 hours after administration

FIG. 13B shows fold change of plasma mSAA concentration 1 day after acute dosing of rhIL-22 (SEQ ID NO: 12).

FIG. 13C shows fold change of plasma mSAA concentration after 5 days of sub-chronic dosing of rhIL-22 (SEQ ID NO: 12).

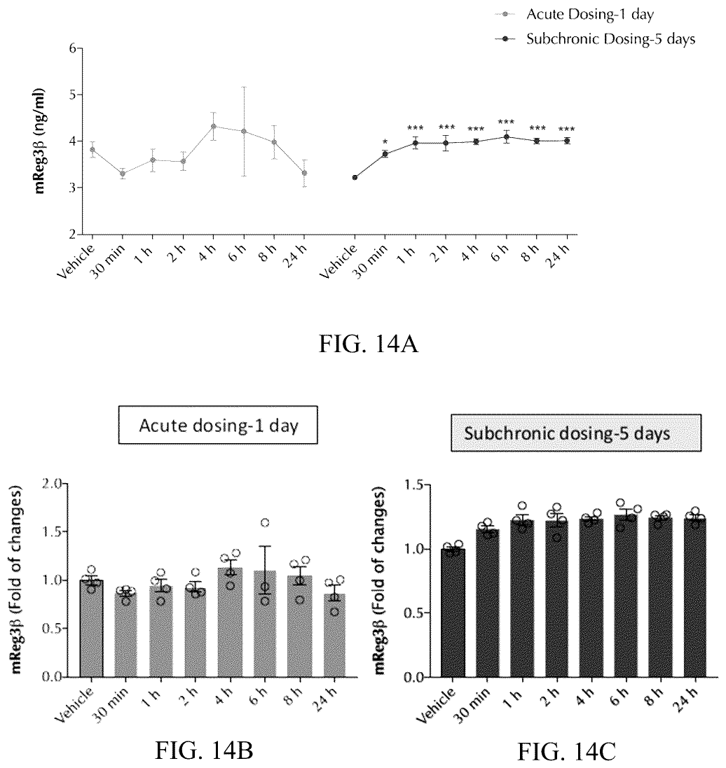

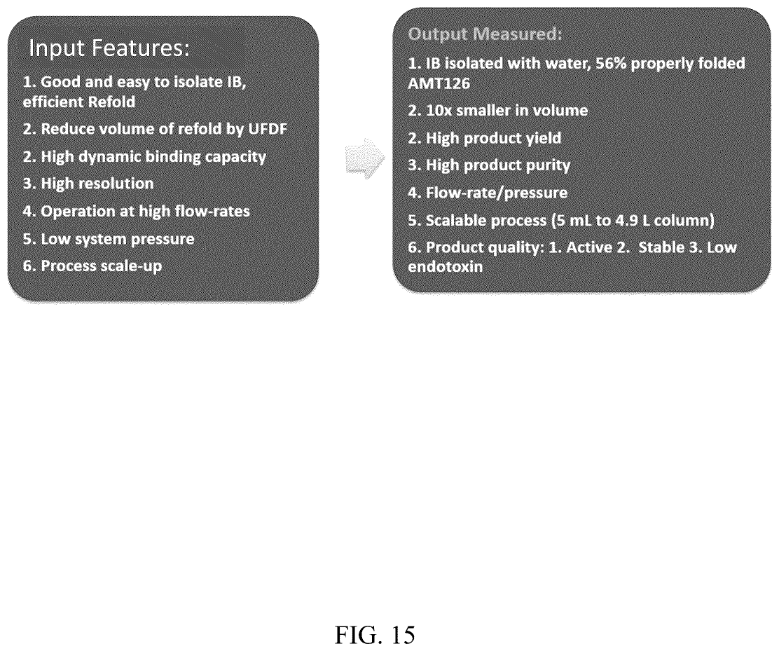

FIG. 14A shows the induced circulating regenerating islet-derived protein 3β (Reg3β) in response to 1 day after acute dosing of rhIL-22 (SEQ ID NO: 12) and after 5 days of sub-chronic dosing rhIL-22 (SEQ ID NO: 12).

FIG. 14B shows fold change of plasma Reg3β concentration 1 day after acute dosing of rhIL-22 (SEQ ID NO: 12).

FIG. 14C shows fold change of plasma Reg3β concentration after 5 days of sub-chronic dosing of rhIL-22 (SEQ ID NO: 12).

These results demonstrate that three potential IL-22 PD biomarkers of target engagement: C reactive protein (CRP), serum amyloid protein A (SAA), and regenerating islet-derived protein 3β (Reg3β) that may be used in studies evaluating the pharmacodynamics (PD) of delivery constructs such as those with SEQ ID NOs: 15 or 17.

### Example 6

### Protein Solubilization

627 g of double-washed inclusion bodies (DWIB) from SEQ ID NO: 15 fermentation were resuspended in 4500 mL of 8M Guanidine/HCl (Gu-HCl), and 50 mM Tris pH at 8.0. Subsequently, 50 mM Tris pH 8.5 was added to complete the volume to 6.0 L. The solution was stirred gently on a stir-plate and 9.225 g of the reducing agent dithiothreitol (DTT) was added. The final solubilized protein (SEQ ID NO: 15) solution of 6 L consisted of 6M Gu-HCl, 50 mM Tris pH 8.0, 10 mM DTT, and ˜1 g DWIB/10 mL buffer. The solubilized protein (SEQ ID NO: 15) solution was incubated at room temperature (RT) while stirred on a magnetic stir-plate, and then centrifuged at 15,970×g for 90 minutes at 4° C. The supernatant comprising the solubilized protein was carefully transferred to a new vessel with a total volume of 5520 mL. The protein concentration determination was performed by Bradford assay and was determined at 15 mg/mL.

The data demonstrate that protein solubilization can be performed using the above procedure.

### Example 7

### Protein Refolding

This example demonstrates protein refolding as part of the purification process as described in the flow chart of FIG. 17.

A refolding solution was carefully researched, developed and optimized (FIG. 18). The optimization process utilized a design of experiments (DOE) of 15 different matrices, 12 variables and 200 refold reactions. Decisions were made by a process of elimination. The initial refold mixture (containing the initial refold solution and the refolded solubilized protein (SEQ ID NO: 15)) is presented in TABLE 2. An optimized refold mixture (containing the optimized refold solution and the refolded solubilized protein (SEQ ID NO: 15)) is presented in TABLE 3.

TABLE 2
Initial refold mixture

```
100 mM Tris pH 8.5 (@ 4° C.)
0.5M arginine
  1M urea
  2 mM EDTA
  0.3 mM GSH
1 mM GSSG
0.2 mg/mL refold concentration
```

TABLE 3
Optimized refold mixture

```
100 mM Tris pH 8.5 (@ 4° C.)
1.0M arginine
10% glycerol
  3 mM L-cysteine
  1 mM cystamine-2HCl
>0.1 mg/mL refold concentration
```

The 105 L refold solution was incubated at 4° C. for 16 hours and then filtered through a flow filter (AkroPac® by Pall corporation or Sartopore 2XLG by Sartorius) using a 0.8/0.2 um membrane and Cole-Parmer® peristaltic pump and tubing.

The solubilized protein (SEQ ID NO: 15) (105 g) from EXAMPLE 6 was added to 100 L of an optimized refold solution prechilled to 4° C.

This process was programmed to one hour using a Cole-Parmer® peristaltic pump set at 73 ml/min. This process took place in a cold room at 4° C.

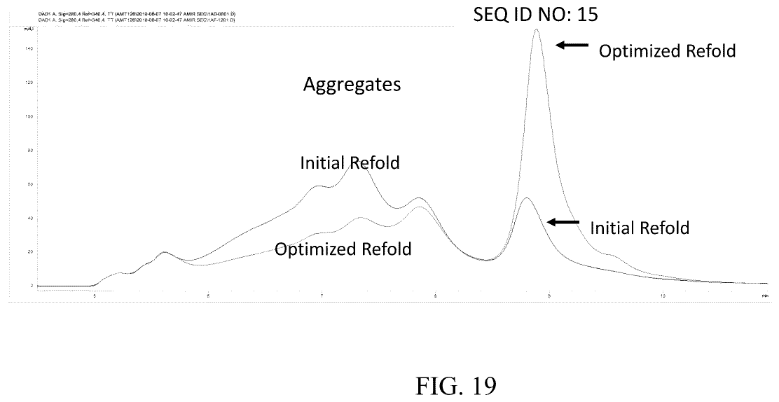

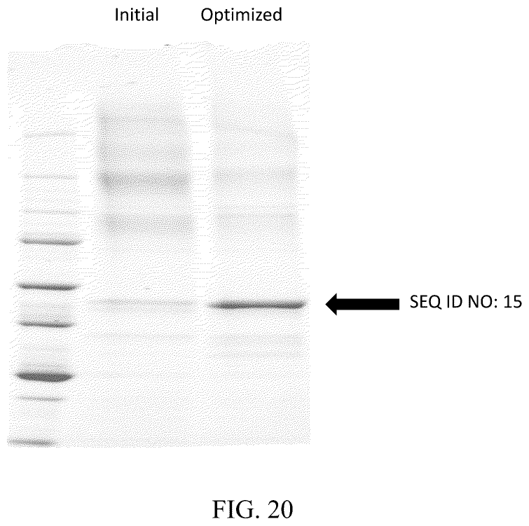

The optimized refold buffer solution produced a larger amount of the SEQ ID NO: 15 relative to aggregates of SEQ ID NO: 15 (FIGS. 19-20). Use of the initial refold solution produced about 10%-15% correctly refolded SEQ ID NO:15. Use of the optimized refold solution produced about 45%-55% correctly refolded SEQ ID NO: 15.

### Example 8

### Protein Concentration and Buffer Exchange by TFF UF/DF

This example demonstrates protein concentration and buffer exchange using tangential flow filtration (TFF) based on ultrafiltration/diafiltration (UF/DF) principles.

Refolded protein (e.g., of SEQ ID NO: 15) was processed by TFF system (Millipore) to concentrate it 10-fold and buffer exchange 5-fold. The process was performed by ultrafiltration/diafiltration (UF/DF) using four 10 kDa MWCO Millipore Ultracell Pellican3, 1.14 m² TFF flat sheet cassettes. The diafiltration buffer consisted of 20 mM Tris-base pH 7.5 and 100 mM NaCl. The final volume at the end of the UF/DF process was 10 L. This material was subsequently filtered using a flow filtration system employing AkroPac® 0.8/0.2 um filter by Pall corporation and Cole-Parmer® peristaltic pump and tubing. Bradford assay was performed to determine the protein concentration, yield, and step recovery. At this point, a quantitative SEC-HPLC was performed to determine the amount of the properly folded protein with SEQ ID NO: 15 present in the refold and UF/DF samples. A commercially available BSA of a known concentration was used as a reference standard from which the standard curve was generated for the Bradford assay. The Agilent 1100 HPLC system and the TSKgel SuperSW3000, 4 μm, 4.6 mm ID×30.0 cm L (Tosoh Bioscience, 18675) column were used. Based on this quantitative assay, the refold efficiency was determined.

The data show that protein refolding can be performed using buffer exchange and TFF UF/DF.

### Example 9

### Protein Chromatography Using the Capture Step NH₂-750F® Resin

This example demonstrates capture steps in protein anion exchange chromatography using the NH₂-750F® resin.

The AKTA Avant 150 or AKTA Pilot FPLC systems from General Electric (GE) were used for protein chromatography. The Tosoh anion exchange resin NH₂-750F® was used to pack a column with a minimum bed-height of at least 20 cm and ensuring a column volume that would facilitate a dynamic binding capacity of 20-25 g/L. A buffer of 20 mM sodium acetate, pH 4.5 or 20 mM sodium citrate, pH 4.5 were used to pack the column. The buffers used were as follow: buffer A: 20 mM Tris pH 7.5, 0.5 M NaCl; buffer B: 20 mM Tris pH 7.5, 2.0 M NaCl. The NH₂-750F column was then cleaned with 0.5 M NaOH solution with a contact time of 30 minutes, and then equilibrated with buffer A for at least 3 column volumes (CV), or until pH and conductivity reach stable lines at the expected values (pH 7.5-pH 7.7, ˜49 mS/cm +/−1 mS/cm).

Prior to loading onto the column, the UF/DF protein (SEQ ID NO: 15) solution was supplemented with 0.4 M NaCl by adding a 5 M stock solution and the conductivity was measured to ensure conductivity of 49 mS/cm +/−2 mS/cm. The protein (SEQ ID NO: 15) solution was loaded onto the column with a flow-rate of a minimum of 5 minutes column residence time, and the flow-through was collected. The column was washed with 3 CV of buffer A or until the absorbance at 280 nm returned, and was stable at baseline, near 0.0 mAU at 280 nm. At this point a linear gradient of 20 CV from 0.0-62.5% B, followed by a step gradient to 100% B for additional 5 CV was performed. Fractions were collected throughout and their volumes were no greater than 0.5 of the column volume. Samples from different fractions were analyzed by SDS-PAGE using the Bio-Rad ChemiDoc™ MP imaging system, and the fractions containing over 90% of SEQ ID NO: 15 were pooled and designated as NH₂-750F-pool. The protein concentration of the NH₂-750F-pool was measured using Thermo Fisher Nanodrop One™, by reading the absorbance at 280 nm (A280) considering extinction coefficient of 1.22 for SEQ ID NO: 15 and a 260/280 nm ratio <0.6. The NH₂-750F-pool contains approximately 1.0 M NaCl.

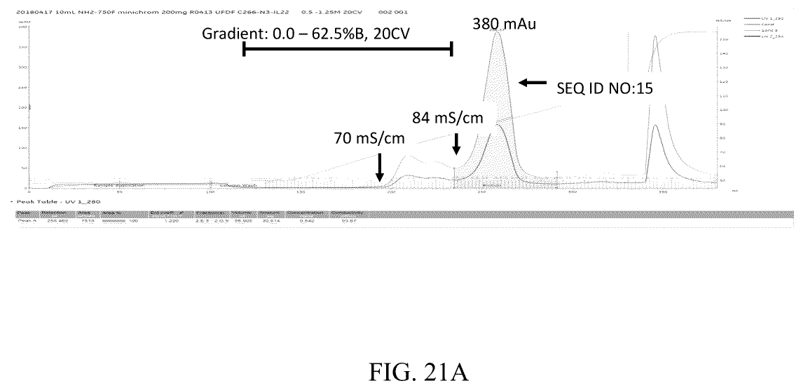

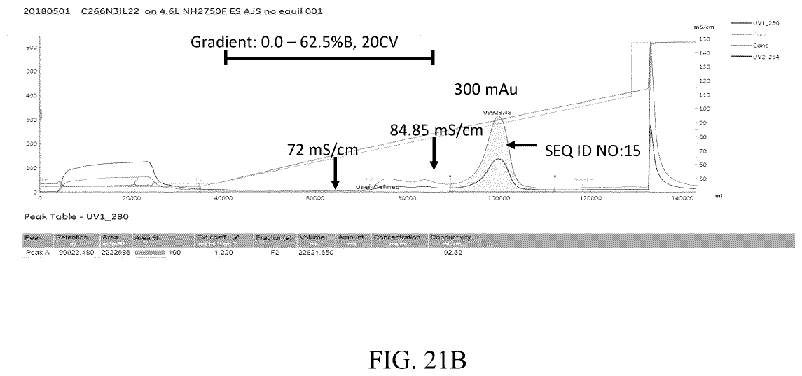

An example chromatogram following NH₂-750F purification of SEQ ID NO: 15 is shown in FIG. 21A for a bed height of 20 cm and a column volume of 10 mL and FIG. 21B for a bed height of 30 cm and a column volume of 4.6 L.

A summary of the NH₂-750F purification is show in TABLE 4 for SEQ ID NO: 15 and TABLE 5 for SEQ ID NO: 14, which was generally produced, refolded, and purified in a manner analogous to SEQ ID NO: 15.

TABLE 4
Summary of SEQ ID NO: 15 NH₂-750F purification
SEQ ID NO: 15

| | |
|---|---|
| Buffer A | 20 mM Tris pH 7.5, 0.5M NaCl |
| Buffer B | 20 mM Tris pH 7.5, 2.0M NaCl |
| Bed Height | Minimum 20 cm |
| Residence Time | 5.0 minutes |
| Gradient | 0.0-62.5% B, 20 CV |
| Binding capacity | 20-25 g/L (Breakthrough at 40 g/L) |
| Purity | 93-96% |
| Recovery | 71-76% |
| Endotoxin | <1.0 EU/mg |

TABLE 5
Summary of SEQ ID NO: 14 NH₂-750F purification
SEQ ID NO: 14

| | |
|---|---|
| Buffer A | 20 mM Tris pH 7.5, 0.7M NaCl, 10% glycerol |
| Buffer B | 20 mM Tris pH 7.5, 2.0M NaCl, 10% glycerol |
| Bed Height | Minimum 20 cm |
| Residence Time | 5.0 minutes |
| Gradient | 0.0-62.5% B, 20 CV |
| Binding | 20-25 g/L |
| capacity | |
| Purity | ≥93-96% |
| Recovery | 80-93% |
| Endotoxin | <1.0 EU/mg |

### Example 10

### Protein Chromatography Using the CaPure® Procedure

This example demonstrates a polishing purification step in protein chromatography using the CaPure® procedure.

The AKTA Avant 150 or AKTA Pilot FPLC systems from General Electric (GE) were used for protein chromatography of proteins of SEQ ID NO: 15 and SEQ ID NO: 14. The Tosoh hydroxyapatite mixed-mode resin CaPure® was used to pack a column for each of SEQ ID NO: 15 and SEQ ID NO: 14 with a minimum bed-height of at least 10 cm and ensuring a column volume that would facilitate a dynamic binding capacity of maximum 20 g/L. The buffers used were as follows for SEQ ID NO: 15: buffer A: 20 mM Tris pH 7.5, 100 mM NaCl, and 1 mM CaCl₂; buffer B: 200 mM sodium phosphate pH 7.0, 100 mM NaCl, and 1 mM CaCl₂. The buffers used were as follows for SEQ ID NO: 14: buffer A: 20 mM Tris pH 7.5, 100 mM NaCl, and 1 mM CaCl₂; buffer B: 200 mM sodium phosphate pH 7.0, 100 mM NaCl, and 1 mM CaCl₂. Each CaPure® column was then cleaned with 0.5 M of NaOH with contact time of 30 minutes or more, and then equilibrated with buffer A for at least 3 column volumes (CV), or until pH and conductivity reach stable lines at the expected values (pH 7.5-pH 7.7, ˜11 mS/cm +/−1 mS/cm). There was no need to treat the NH₂-750F-pool prior to loading it onto the column. This significantly reduced protein loss, time, and resources. The NH₂-750F pool was loaded onto the column with a flow-rate of a minimum of 5 minutes column residence time, and the flow-through was collected. The column was washed with 3 CV of buffer A or until the absorbance at 280 nm returned, and was stable at baseline, near 0.0 mAu. At this point, a linear gradient of 25 CV from 0-25% B, followed by a step gradient to 100% B for additional 5 CV was performed. Fractions were collected throughout and their volumes were ranging from 0.36 to 1.43 of the column volume, depending on the elution profile. Samples from different fractions were analyzed by SDS-PAGE using the ChemiDoc™ MP imaging system, and the fractions containing over 95% of SEQ ID NO: 15 or SEQ ID NO: 14 were pooled and designated as CaPure-pool. The protein concentration of the CaPure-pool was measured using Thermo Fisher Nanodrop One™, by reading the absorbance at 280 nm (A280) considering extinction coefficient of 1.22 for SEQ ID NO: 15 and a 260/280 nm ratio <0.6.

An exemplary chromatogram following CaPure® purification of SEQ ID NO: 15 is shown in FIG. 22A for a bed height of 10 cm and column volume of 5mL and FIG. 22B for a bed height of 21 cm and a column volume of 800 mL.

A summary of the CaPure® purification is show in TABLE 6 for SEQ ID NO: 15 and TABLE 7 for SEQ ID NO: 14.

TABLE 6
Summary of SEQ ID NO: 15 CaPure ® purification
SEQ ID NO: 15

| | |
|---|---|
| Buffer A | 20 mM Tris pH 7.5, 100 mM NaCl, 1 mM CaCl₂ |
| Buffer B | 200 mM sodium phosphate pH 7.0, 100 mM NaCl, |
| | 1 mM CaCl₂ |
| Bed Height | 20 cm |
| Residence | 5.0 minutes |
| Time | |
| Gradient | 0.0-25% B, 25 CV |
| Binding | 20 g/L |
| capacity | |
| Purity | >98% |
| Recovery | >92% |
| Endotoxin | <1.0 EU/mg |

TABLE 7
Summary of SEQ ID NO: 14 CaPure ® purification
SEQ ID NO: 14

| | |
|---|---|
| Buffer A | 20 mM Tris pH 7.5, 100 mM NaCl, 1 mM CaCl₂ |
| Buffer B | 200 mM sodium phosphate pH 7.0, 100 mM NaCl, |
| | 1 mM CaCl₂ |
| Bed Height | 20 cm |
| Residence | 5.0 minutes |
| Time | |
| Gradient | 0.0-25% B, 25 CV |
| Binding | 20 g/L |
| capacity | |
| Purity | >98% |
| Recovery | >92% |
| Endotoxin | <1.0 EU/mg |

### Example 11

### Protein Concentration by TFF UF/DF

This example demonstrates protein formulation by TFF UF/DF procedures.

The CaPure® pool was concentrated by a TFF system (Pall corporation) to a final concentration of 20 mg/mL followed by 5-fold buffer exchange. The process was performed by ultrafiltration/diafiltration (UF/DF) using three 10 kDa MWCO Millipore Pellican3, 0.114 m² TFF flat sheet cassettes. The diafiltration buffer consisted of 10 mM sodium phosphate pH7.0, 100 mM NaCl. The formulated SEQ ID NO: 15 was subsequently filtered using a flow filtration system employing AkroPac® 0.8/0.2 um filter by Pall corporation and Cole-Parmer® peristaltic pump and tubing. The formulated SEQ ID NO: 15 was then stored in aliquots at −80° C. and the following analyses were performed to ensure its biophysical quality: (1) Protein (e.g., SEQ ID NO: 15) concentration at 20 mg/mL by measuring the absorbance at 280 nm with 260/280 ratio <0.6 using Thermo Fisher Nanodrop One™ and considering protein extinction coefficient of 1.22; (2) LAL endotoxin levels below 1.0 Eu/mg measured by Charles River Laboratories equipment; (3) Purity by SEC-HPLC above 97% purity (typically >98%) using TSKgel GW3000SWXL, 5 μm, 7.8 mm ID×30.0 cm L column (Tosoh Bioscience, 8541) (4) SDS-PAGE analysis using the Bio-Rad gel apparatus and its associated ChemiDoc™ MP imaging system.

### Example 12

### Evaluation of In Vivo Activity of Purified Fusion Molecules

This example demonstrates methods for verifying the proper folding of the fusion molecules with regard to their ability to carry a biologically active cargo across an intact epithelium.

The fusion protein with SEQ ID NO: 15 is expressed by E. coli and collected from inclusion bodies and folded using a shuffle exchange buffer system as described in EXAMPLE 8 above. The resulting material is purified according to the methods described in EXAMPLE 9 and EXAMPLE 10, and as summarized for example in TABLES 4-7, depending on the molecular characteristics of the fusion molecule. The preparation has a protein purity of ˜98% based upon SDS PAGE. Epithelial cells are treated with the fusion molecule having SEQ ID NO: 15 at concentrations of 25 nM and 250 nM. Compared to untreated matched cells, SEQ ID NO: 15 treated cells produce a dose-dependent decrease in cell number as assessed by flow cytometry of live/dead cells). Values represent n=4±standard deviation.

### Example 13

### Scale-Up of Fusion Protein Purification

This example demonstrates a scale-up of a purification method using the fusion protein of SEQ ID NO: 15 (FIGS. 7A and 7B).

The fusion protein with SEQ ID NO: 15 was purified on two different scales.

The first purification was carried out using a chromatography column with a volume of 10 mL packed with NH₂-750F® resin and the following parameters: Bed height: 20 cm, Residence time: 5 min (flow rate: 2 mL/min), buffer A: 20 mM Tris pH 7.5, 0.5 M NaCl, buffer B: 20 mM Tris pH 7.5, 2 M NaCl, and using the following gradient: 0.0-62.5% B for 20 column volumes (CVs). The fusion protein with SEQ ID NO: 15 was obtained with a purity of 93-96%, a recovery of >71%, and endotoxin levels <1.0 EU/mg.

The second purification was carried out using a chromatography column with a volume of 4.6 L packed with NH₂-750F® resin and the following parameters: Bed height: 30 cm, Residence time: 11.55 min (flow rate: 400 mL/min), buffer A: 20 mM Tris pH 7.5, 0.5 M NaCl, buffer B: 20 mM Tris pH 7.5, 2 M NaCl, and using the following gradient: 0.0-62.5% B for 20 column volumes (CVs). The fusion protein with SEQ ID NO: 15 was obtained with a purity of 93-96%, a recovery of >71%, and endotoxin levels <1.0 EU/mg.

### Example 14

### Consistency Between Process Step Recoveries

The purification process was carried out to purify the fusion protein of SEQ ID NO: 15 four times (lots 1-4), and the recovery of this fusion protein was assessed after each stage of the purification process (TABLE 8). There was a high degree of consistency in the recovery of the fusion protein between these replicates.

TABLE 8
Recovery of the fusion protein of SEQ ID NO:
15 in 4 replicate purification processes

| Process | Process | | | | | Average |
|---|---|---|---|---|---|---|
| step | description | Lot 1 | Lot 2 | Lot 3 | Lot 4 | (%) |
| 2 | Refold | 98.60% | 94% | 92.40% | 85% | 92 |
| | UF/DF | | | | | |
| 3 | NH₂-750F | 68.60% | 66.30% | 76% | 74.50% | 71.35% |
| 4 | CaPure | 99% | 96.40% | 100% | 94.80% | 97.55 |
| 5 | Formulation | 97.80% | 100% | 98.75% | 100% | 99.14 |
| | UF/DF | | | | | |

These data demonstrate that the methods and compositions of the present disclosure allowed the production and purification of therapeutic fusion protein on a large scale, and that the herein disclosed methods and compositions provided high consistency during scale-up.

All of the methods disclosed and claimed herein may be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### Example 15

### Comparison of SEQ ID NO: 15 and SEQ ID NO: 17

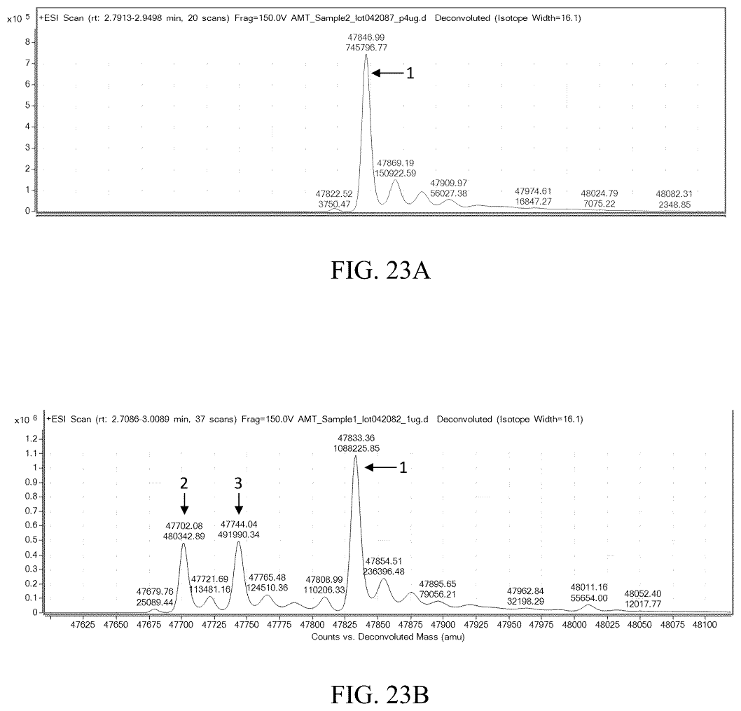

Liquid chromatography-mass spectrophotometry (LC-MS) was performed on purified compositions representing SEQ ID NO: 15 and SEQ ID NO: 17, and the results are represented in FIG. 23B and FIG. 23A, respectively. The composition of SEQ ID NO: 17 was produced, refolded, and purified in a manner analogous to that described above in connection with the composition of SEQ ID NO: 15.

### Example 16

### In Vivo Assessment of Delivery Constructs

This example describes an in vivo assessment of a delivery construct having the sequence of SEQ ID NO: 15 compared to the vehicle.

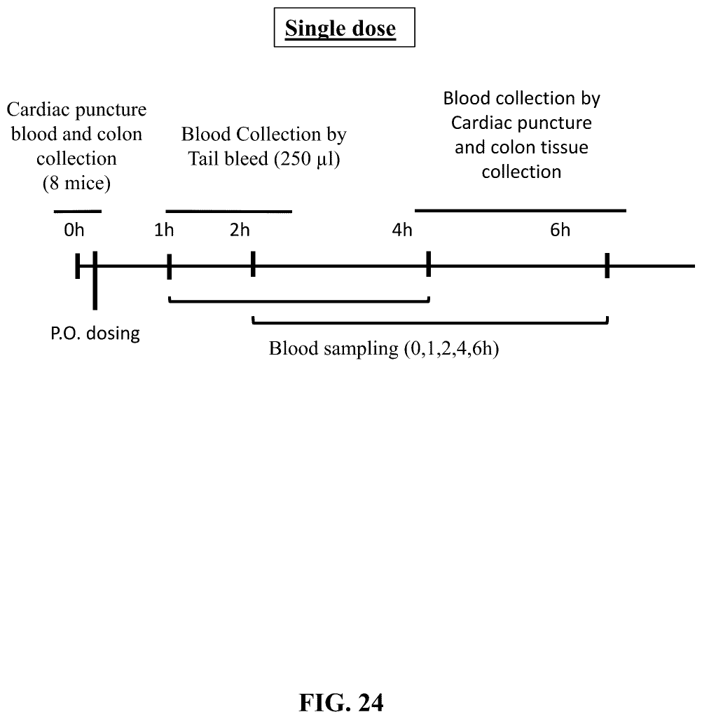

FIG. 24 shows a (single-dose) study overview for assessing transport of the delivery construct having the sequence of SEQ ID NO: 15 across the gut epithelium and into circulation after oral (abbreviated herein as P.O.) delivery of the construct. The animals used in these experiments were CD-1 male mice without fasting. A primary study endpoint was total plasma IL-22 exposure, and a secondary endpoint were plasma biomarkers, including IL-22 binding protein (e.g., IL-22BP).

TABLE 9 provides details regarding the experimental setup, including information regarding the liquid (unformulated) delivery construct and the vehicle.

TABLE 9
Experimental Setup of the In Vivo Study

| | | | | | | | Time- |
|---|---|---|---|---|---|---|---|
| | Test | | Formu- | Dose | | N (4/ | points |
| Group | Article | Vehicle | lation | (mpk) | Route | two TP) | (hours) |
| 1 | Vehicle | PBS, | Aq. bolus | na | P.O. | 4 | 1 |
| | | pH 7 | | | | | |
| 2 | SEQ ID | PBS, | Aq. bolus | 90 | P.O. | 8 (4 * 2) | 1, 2, 4, 6 |
| | NO: 15 | pH 7 | | | | | |
| | (liquid) | | | | | | |

TABLE 10 summarizes properties of the SEQ ID NO: 15 construct as delivered in this experiment.

TABLE 10
Properties of Delivery Construct as Delivered in Liquid Form

| | | Cmax | Tmax | |
|---|---|---|---|---|
| Groups | Groups | (pg/mL) | (hr) | AUC (0-6 hr) |
| SEQ ID NO: | n/a | 24488 | 2 | 58126 |
| 15-Liquid | | | | |

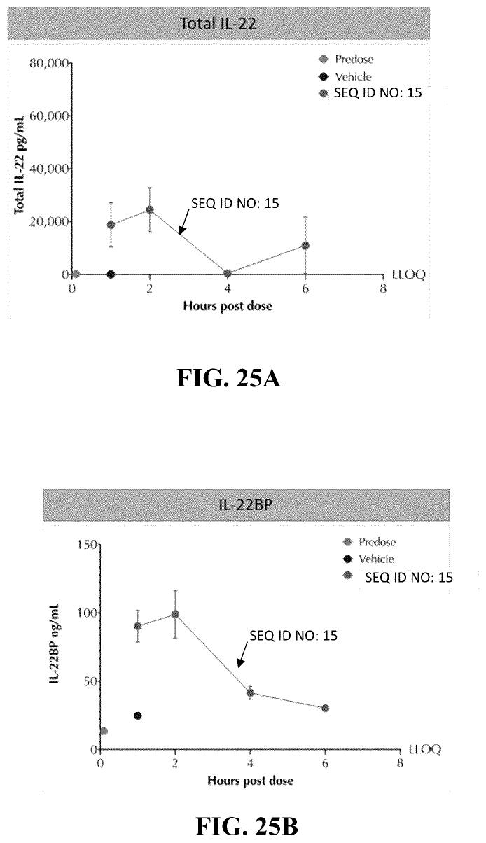

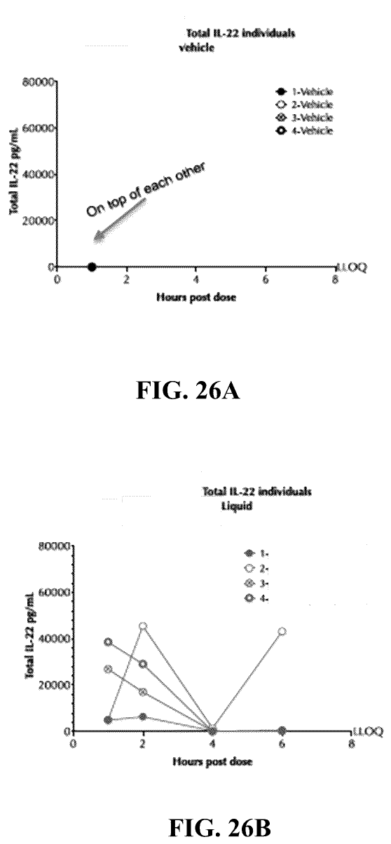

The total plasma IL-22 exposure was measured at the respective time points shown in TABLE 9. FIG. 25A shows total systemic IL-22 exposure (in pg/mL) and FIG. 25B shows IL-22BP exposure (in pg/mL) at the various time points where the blood samples and measurements were taken. FIG. 26A-FIG. 26B show individual measurements and data points for each group tested in this study.

It was observed that orally administered delivery construct having the sequence set forth in SEQ ID NO: 15 provided IL-22 in the plasma.

### Example 17

### Spacer Length and Coupling of Payload to the N- or C-Terminus of a Carrier Does not Significantly Affect a Payload's Biological Activity

This example shows that amino acid linkers of various lengths and the coupling of a heterologous payload to the N-terminus of a carrier does not significantly impact the payload's ability to bind its target when included into a delivery construct. The amino acid linkers examined were SEQ ID NO: 13 (GGGGSGGGGSGGGGS), SEQ ID NO: 28 (GGGGS), and SEQ ID NO: 31 (GGGGSGGGGSGGGGSGGGGSGGGGS).

The IL-22 receptor dimerization assay was performed by seeding DiscoverX HEK293 cells and incubating the cells for 16 h (5,000 cells per well) using the shown concentrations of agonist (delivery construct containing the IL-22 payload). The endpoint luminescence was read on a plate reader using PathHunter® eXpress IL22RA1/IL10RB Dimerization Assay.

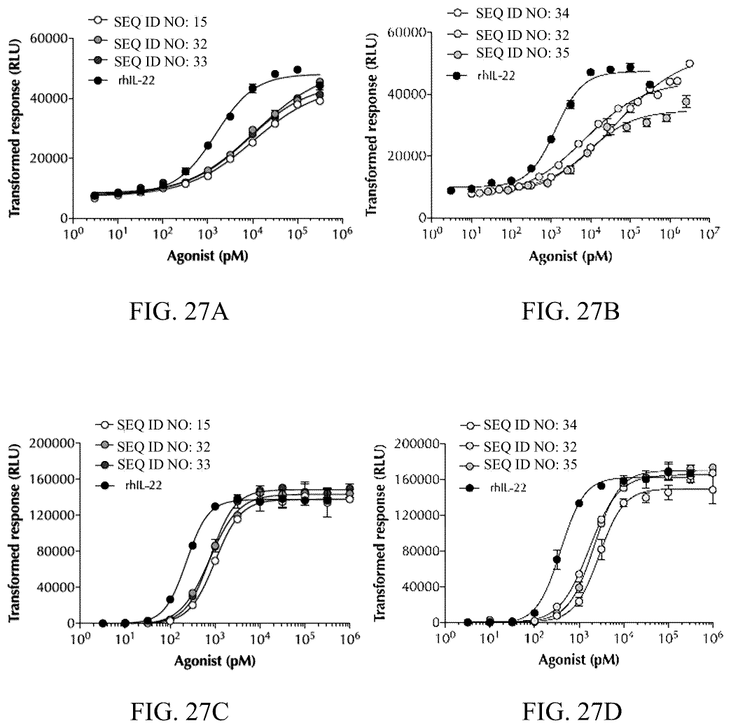

FIG. 27A shows that the length of amino acid spacers with SEQ ID NOs: 13, 28, and 31 did not impact the ability of IL-22 (SEQ ID NO: 11) when included in the delivery constructs with SEQ ID NOs: 15, 32, and 33 to induce IL-22 receptor dimerization. The induction of receptor dimerization of control recombinant human IL-22 (rhIL-22, SEQ ID NO: 12) is shown by the black curve.

FIG. 27B shows that coupling of the IL-22 payload (SEQ ID NO: 11) to the N- or to the C-terminus of a carrier comprising amino acid residues 1-266 of SEQ ID NO: 1 via the spacer with SEQ ID NO: 28 did not significantly change the ability of the delivery constructs with SEQ ID NOs: 32, 34, and 35 to induce IL-22 receptor dimerization. The induction of receptor dimerization of control recombinant human IL-22 (rhIL-22) is shown by the black curve.

The pSTAT3 activation assay was conducted using Colo205 cells incubated with 10 μL of agonist (the respective delivery construct or IL-22 control) having the various concentrations for 15 min. The extent of pSTAT3 activation was then read using MSD STAT3 plates (Cat. No. N450SMA-1).

FIG. 27C shows that the length of amino acid spacers with SEQ ID NOs: 13, 28, and 31 did not impact the ability of IL-22 (SEQ ID NO: 11) when included in the delivery constructs with SEQ ID NOs: 15, 32, and 33 to induce pSTAT3 activation. The pSTAT3 activation of control recombinant human IL-22 (rhIL-22, SEQ ID NO: 12) is shown by the black curve.

FIG. 27D shows that coupling of the IL-22 payload (SEQ ID NO: 11) to the N- or to the C-terminus of a carrier comprising amino acid residues 1-266 of SEQ ID NO: 1 via the spacer with SEQ ID NO: 28 did not significantly change the ability of the delivery constructs with SEQ ID NOs: 32, 34, and 35 to induce pSTAT3 activation. The pSTAT3 activation of control recombinant human IL-22 (rhIL-22) is shown by the black curve.

1-3. (canceled)

4. A delivery construct comprising a carrier coupled to an IL-22 payload, wherein the carrier consists of an amino acid sequence set forth in SEQ ID NO: 7 or SEQ ID NO: 9, or an amino acid sequence having at least 90% sequence identity thereto, wherein the delivery construct is capable of transcytosing across a polarized epithelial cell.

5. The delivery construct of claim 4, wherein the carrier has a glutamic acid at position 3 and an alanine at position 4.

6. The delivery construct of claim 4, wherein the carrier is 266 amino acids in length.

7. The delivery construct of claim 4, wherein the carrier consists of the amino acid sequence set forth in SEQ ID NO: 7 or SEQ ID NO: 9, or an amino acid sequence having at least 95% sequence identity thereto.

8. The delivery construct of claim 4, wherein the carrier consists of the amino acid sequence set forth in SEQ ID NO: 7 or SEQ ID NO: 9, or an amino acid sequence having at least 99% sequence identity thereto.

9. The delivery construct of claim 4, wherein the carrier consists of the amino acid sequence set forth in SEQ ID NO: 7.

10. The delivery construct of claim 4, wherein the carrier consists of the amino acid sequence set forth in SEQ ID NO: 9.

11. The delivery construct of claim 4, wherein the IL-22 comprises an amino acid sequence set forth in SEQ ID NO: 11.

12. The delivery construct of claim 4, wherein the carrier is coupled non-covalently to the IL-22 payload.

13. The delivery construct of claim 4, wherein the carrier is coupled covalently to the IL-22 payload.

14. The delivery construct of claim 13, wherein the carrier is coupled covalently to the IL-22 payload via a spacer.

15. The delivery construct of claim 14, wherein the spacer consists of an amino acid sequence set forth in SEQ ID NO: 13.

16. A pharmaceutical composition comprising the delivery construct of claim 4, wherein the pharmaceutical composition is formulated for oral administration.

17. A method of treating an inflammatory disease in a subject, the method comprising administering to the subject an effective amount of a delivery construct of claim 4.

18. The method of claim 17, wherein the inflammatory disease is hepatitis, obesity, fatty liver disease, liver inflammation, or pancreatitis, Crohn's disease, ulcerative colitis, pouchitis, proctitis, multiple sclerosis, systemic lupus erythematosus, graft versus host disease, rheumatoid arthritis, or psoriasis.

19. The method of claim 18, wherein the disease is Crohn's disease or ulcerative colitis.

20. A delivery construct comprising a carrier, wherein the carrier: has its C-terminus at any one of positions 195 to 347; has a glutamic acid at position 3 and an alanine at position 4; and is capable of transcytosing across a polarized epithelial cell.


SEQUENCE LISTING (41 sequences)

SEQ ID NO: 1 - protein, 634 aa, synthetic construct
  REGION 1..634: note = Description of Artificial Sequence: Synthetic polypeptide
VEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAADILSLFCPDADKSCVASNNDQANINIESRSGRSYLPENRAVITPQGVTNWTYQELEATHQALTREGYVFVGYHGTNHVAAQTIVNRIAPVPRGNNTENEEKWGGLYVATHAEVAHGYARIKEGTGEYGLPTRAERDARGVMLRVYIPRASLERFYRTNTPLENAEEHITQVIGHSLPLRNEAFTGPESAGGEDETVIGWDMAIHAVAIPSTIPGNAYEELAIDEEAVAKEQSISTKPPYKERKDELK

SEQ ID NO: 2 - protein, 386 aa, synthetic construct
  REGION 1..386: note = Description of Artificial Sequence: Synthetic polypeptide
VEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQA

SEQ ID NO: 3 - protein, 266 aa, synthetic construct
  REGION 1..266: note = Description of Artificial Sequence: Synthetic polypeptide
VEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKG

SEQ ID NO: 4 - protein, 386 aa, synthetic construct
  REGION 1..386: note = Description of Artificial Sequence: Synthetic polypeptide
LEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQA

SEQ ID NO: 5 - protein, 266 aa, synthetic construct
  REGION 1..266: note = Description of Artificial Sequence: Synthetic polypeptide
LEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKG

SEQ ID NO: 6 - protein, 387 aa, synthetic construct
  REGION 1..387: note = Description of Artificial Sequence: Synthetic polypeptide
MVEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQA

SEQ ID NO: 7 - protein, 267 aa, synthetic construct
  REGION 1..267: note = Description of Artificial Sequence: Synthetic polypeptide
MVEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKG

SEQ ID NO: 8 - protein, 387 aa, synthetic construct
  REGION 1..387: note = Description of Artificial Sequence: Synthetic polypeptide
MLEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQA

SEQ ID NO: 9 - protein, 267 aa, synthetic construct
  REGION 1..267: note = Description of Artificial Sequence: Synthetic polypeptide
MLEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKG

SEQ ID NO: 10 - protein, 179 aa, synthetic construct
  REGION 1..179: note = Description of Artificial Sequence: Synthetic polypeptide
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 11 - protein, 146 aa, synthetic construct
  REGION 1..146: note = Description of Artificial Sequence: Synthetic polypeptide
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 12 - protein, 147 aa, synthetic construct
  REGION 1..147: note = Description of Artificial Sequence: Synthetic polypeptide
MAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 13 - protein, 15 aa, synthetic construct
  REGION 1..15: note = Description of Artificial Sequence: Synthetic peptide
GGGGSGGGGSGGGGS

SEQ ID NO: 14 - protein, 548 aa, synthetic construct
  REGION 1..548: note = Description of Artificial Sequence: Synthetic polypeptide
MLEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAGGGGSGGGGSGGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 15 - protein, 428 aa, synthetic construct
  REGION 1..428: note = Description of Artificial Sequence: Synthetic polypeptide
MVEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGGGGGSGGGGSGGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 16 - protein, 548 aa, synthetic construct
  REGION 1..548: note = Description of Artificial Sequence: Synthetic polypeptide
MVEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAGGGGSGGGGSGGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 17 - protein, 428 aa, synthetic construct
  REGION 1..428: note = Description of Artificial Sequence: Synthetic polypeptide
MLEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGGGGGSGGGGSGGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 18 - protein, 547 aa, synthetic construct
  REGION 1..547: note = Description of Artificial Sequence: Synthetic polypeptide
VEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAGGGGSGGGGSGGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 19 - protein, 547 aa, synthetic construct
  REGION 1..547: note = Description of Artificial Sequence: Synthetic polypeptide
LEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAGGGGSGGGGSGGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 20 - protein, 427 aa, synthetic construct
  REGION 1..427: note = Description of Artificial Sequence: Synthetic polypeptide
VEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGGGGGSGGGGSGGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 21 - protein, 427 aa, synthetic construct
  REGION 1..427: note = Description of Artificial Sequence: Synthetic polypeptide
LEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGGGGGSGGGGSGGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 22 - protein, 634 aa, synthetic construct
  REGION 1..634: note = Description of Artificial Sequence: Synthetic polypeptide
LEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAADILSLFCPDADKSCVASNNDQANINIESRSGRSYLPENRAVITPQGVTNWTYQELEATHQALTREGYVFVGYHGTNHVAAQTIVNRIAPVPRGNNTENEEKWGGLYVATHAEVAHGYARIKEGTGEYGLPTRAERDARGVMLRVYIPRASLERFYRTNTPLENAEEHITQVIGHSLPLRNEAFTGPESAGGEDETVIGWDMAIHAVAIPSTIPGNAYEELAIDEEAVAKEQSISTKPPYKERKDELK

SEQ ID NO: 23 - protein, 635 aa, synthetic construct
  REGION 1..635: note = Description of Artificial Sequence: Synthetic polypeptide
MLEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAADILSLFCPDADKSCVASNNDQANINIESRSGRSYLPENRAVITPQGVTNWTYQELEATHQALTREGYVFVGYHGTNHVAAQTIVNRIAPVPRGNNTENEEKWGGLYVATHAEVAHGYARIKEGTGEYGLPTRAERDARGVMLRVYIPRASLERFYRTNTPLENAEEHITQVIGHSLPLRNEAFTGPESAGGEDETVIGWDMAIHAVAIPSTIPGNAYEELAIDEEAVAKEQSISTKPPYKERKDELK

SEQ ID NO: 24 - protein, 427 aa, synthetic construct; identical to SEQ ID NO: 20
  REGION 1..427: note = Description of Artificial Sequence: Synthetic polypeptide

SEQ ID NO: 25 - protein, 427 aa, synthetic construct; identical to SEQ ID NO: 21
  REGION 1..427: note = Description of Artificial Sequence: Synthetic polypeptide

SEQ ID NO: 26 - protein, 649 aa, synthetic construct
  REGION 1..649: note = Description of Artificial Sequence: Synthetic polypeptide
  MOD_RES 1: note = V or L
  MOD_RES 3: note = E or D
  MOD_RES 4: note = A or E
  MOD_RES 6: note = N or K
  MOD_RES 16: note = S or L
  MOD_RES 21: note = P or L
  MOD_RES 24: note = P or Q
  MOD_RES 30: note = S or F
  MOD_RES 33: note = S or G
  MOD_RES 56: note = K or M
  MOD_RES 59: note = D or G
  MOD_RES 67: note = I or F
  MOD_RES 73: note = V or I
  MOD_RES 81: note = N or S
  MOD_RES 90: note = H or N
  MOD_RES 101: note = T or M
  MOD_RES 104: note = Y or F
  MOD_RES 108: note = E or D
  MOD_RES 109: note = G or S
  MOD_RES 112: note = A or T
  MOD_RES 114: note = N or H
  MOD_RES 118: note = P or I
  MOD_RES 119: note = I or P
  MOD_RES 131: note = V or I
  MOD_RES 134: note = L or I
  MOD_RES 137: note = Q or K
  MOD_RES 160: note = K or E
  MOD_RES 161: note = T or N
  MOD_RES 166: note = S or F
  MOD_RES 168: note = S or A
  MOD_RES 174: note = H or Q
  MOD_RES 175..185: note = This region may encompass one of the following sequences: "N", "S", "SIAKQS", or "SIAKQSIAKQS"
  MOD_RES 196: note = K or N
  MOD_RES 199: note = Q, E, or H
  MOD_RES 201: note = E, N, or D
  MOD_RES 203: note = S or A
  MOD_RES 210: note = H or N
  MOD_RES 212: note = H, L, F, or R
  MOD_RES 214: note = G or T
  MOD_RES 215: note = L or S
  MOD_RES 216: note = A or P
  MOD_RES 217: note = L, E, or K
  MOD_RES 218: note = C or V
  MOD_RES 219: note = W, V, or T
  MOD_RES 221: note = V or absent
  MOD_RES 222: note = P or absent
  MOD_RES 223: note = M, I, L, or absent
  MOD_RES 224: note = D or absent
  MOD_RES 225: note = A or absent
  MOD_RES 226: note = I or absent
  MOD_RES 227: note = Y or C
  MOD_RES 228: note = N or F
  MOD_RES 229: note = Y or F
  MOD_RES 230: note = I or E
  MOD_RES 231: note = T or D
  MOD_RES 232: note = Q or P
  MOD_RES 233: note = Q, E, or A
  MOD_RES 234: note = N, L, or Q
  MOD_RES 237: note = L or Y
  MOD_RES 239: note = D or E
  MOD_RES 240: note = N or D
  MOD_RES 242: note = F, H, or Y
  MOD_RES 245: note = S or A
  MOD_RES 247: note = E or K
  MOD_RES 252: note = T or I
  MOD_RES 254: note = K, E, or G
  MOD_RES 255: note = V or A
  MOD_RES 257: note = T or M
  MOD_RES 262: note = I or M
  MOD_RES 266: note = P, T, or A
  MOD_RES 275: note = K, Q, or N
  MOD_RES 276: note = G or K
  MOD_RES 279: note = M or I
  MOD_RES 280: note = S or E
  MOD_RES 281: note = A or T
  MOD_RES 298: note = S or G
  MOD_RES 303: note = D or Y
  MOD_RES 305: note = T, P, or Q
  MOD_RES 309: note = S or Q
  MOD_RES 311: note = A or V
  MOD_RES 313: note = Q or N
  MOD_RES 316: note = N or Q
  MOD_RES 322: note = V or L
  MOD_RES 326: note = I or M
  MOD_RES 329: note = S or T
  MOD_RES 331: note = L or I
  MOD_RES 334: note = V or I
  MOD_RES 340: note = D, E, or H
  MOD_RES 341: note = E or G
  MOD_RES 343: note = E or A
  MOD_RES 345: note = E or A
  MOD_RES 347: note = A or T
  MOD_RES 351: note = S, D, or T
  MOD_RES 352: note = D or A
  MOD_RES 353: note = L or I
  MOD_RES 355: note = R or Q
  MOD_RES 359: note = N or D
  MOD_RES 363: note = M or V
  MOD_RES 365: note = T or I
  MOD_RES 370: note = V or I
  MOD_RES 381: note = H or E
  MOD_RES 384: note = G or L
  MOD_RES 386: note = T or I
  MOD_RES 393: note = G or S
  MOD_RES 403: note = F or L
  MOD_RES 404: note = C or Y
  MOD_RES 407: note = A or T
  MOD_RES 409: note = K, E, or G
  MOD_RES 410: note = S, P, or H
  MOD_RES 414: note = S or L
  MOD_RES 415: note = N or D
  MOD_RES 416: note = N or S
  MOD_RES 423: note = I or V
  MOD_RES 433: note = P or L
  MOD_RES 441: note = P or Q
  MOD_RES 453: note = E or D
  MOD_RES 454: note = A or T
  MOD_RES 455: note = T or K
  MOD_RES 458: note = A or T
  MOD_RES 461: note = R or Q
  MOD_RES 463: note = G or D
  MOD_RES 475: note = V or A
  MOD_RES 479: note = T, S, or N
  MOD_RES 485: note = A, S, or T
  MOD_RES 491: note = N or S
  MOD_RES 492: note = N or D
  MOD_RES 495: note = N, S, or K
  MOD_RES 497: note = E, R, or K
  MOD_RES 498: note = K, A, or E
  MOD_RES 502: note = L or V
  MOD_RES 505: note = A or S
  MOD_RES 507: note = H or D
  MOD_RES 509: note = E or S
  MOD_RES 510: note = V or L
  MOD_RES 511: note = A or N
  MOD_RES 512: note = H or Y
  MOD_RES 513: note = G or R
  MOD_RES 515: note = A or T
  MOD_RES 517: note = I or L
  MOD_RES 518: note = K or Q
  MOD_RES 519: note = E or K
  MOD_RES 522: note = G or A
  MOD_RES 523: note = E, D, or N
  MOD_RES 524: note = Y, G, A, or N
  MOD_RES 525: note = G or E
  MOD_RES 526: note = L or G
  MOD_RES 527: note = P or L
  MOD_RES 529: note = R, P, or T
  MOD_RES 530: note = A or E
  MOD_RES 531: note = E or K
  MOD_RES 532: note = R, Q, or K
  MOD_RES 533: note = D, K, or E
  MOD_RES 534: note = A, T, or S
  MOD_RES 540: note = R or K
  MOD_RES 543: note = I or L
  MOD_RES 544: note = P or H
  MOD_RES 545: note = R or Q
  MOD_RES 554: note = T or I
  MOD_RES 556: note = T, A, or I
  MOD_RES 557: note = P or D
  MOD_RES 560: note = N or K
  MOD_RES 561: note = A or E
  MOD_RES 562: note = E, R, or D
  MOD_RES 563: note = E, N, or R
  MOD_RES 564: note = H or L
  MOD_RES 565: note = I or V
  MOD_RES 566: note = T or E
  MOD_RES 567: note = Q, R, H, or D
  MOD_RES 572: note = S or P
  MOD_RES 583: note = P or T
  MOD_RES 584: note = E or D
  MOD_RES 585: note = S, A, or R
  MOD_RES 586: note = A, E, or V
  MOD_RES 587: note = G, E, or D
  MOD_RES 589: note = E or S
  MOD_RES 590: note = D or N
  MOD_RES 593: note = V or A
  MOD_RES 598: note = M or I
  MOD_RES 601: note = H or Y
  MOD_RES 602: note = A or G
  MOD_RES 613: note = A or S
  MOD_RES 615: note = E or A
  MOD_RES 616: note = E, A, Q, G, V, or R
  MOD_RES 618: note = A, P, or T
  MOD_RES 619: note = I, T, or P
  MOD_RES 620: note = D or A
  MOD_RES 624..629: note = This region may encompass one of the following sequences: "V" or "VVKEAI"
  MOD_RES 631: note = K or E
  MOD_RES 637: note = T, A, or P
  MOD_RES 644: note = R, Q, or H
  MOD_RES 645: note = K or absent
  REGION 1..649: note = See specification as filed for detailed description of substitutions and preferred embodiments
XEXXLXIFDECRSPCXLTPEXGKXIQSKLXIPXDVVLDEGVLYYSMTINDEQNDIXDEXKGESIITXGEFATXRATRHYVXQDAPFGVIXLDITTENGTKXYSXNRKXXEFXIXWLVXXGEDSPASIKISXDEXDQXRNIIEVPKLYSIDLDNQTLEQWXXQGNVXFXVTRPEXXXXXXXXXXXXIAISWPSVSYXAAXKXGXRHKRWAXWXTXXXXXXLXXXXXXXXXXXXXXCTXGXXWXGGXYXTVAGXPXXIXVKQGXEQKXVEQRIHFSXXNAXXXLAAHRVCGVPLETLARXRKPRXLXDDLXCXYXAQXIVSLFXATRXLFXHXDSXFTLNLXXQXPXVXERLXXXRXINEXNPGXVXQVLTXARQIYNDYVTXHPXLXPEQTSAXAQAADILSLXXPDXDXXCVAXXXDQANINXESRSGRSYLXENRAVITXQGVTNWTYQELXXXHQXLTXEXYVFVGYHGTNHXAAQXIVNRIXPVPRGXXTEXEXXWGGXYVXTXAXXXXXYXRXXXGTXXXXXXTXXXXXXRGVMLXVYXXXASLERFYRXNXXLEXXXXXXXXVIGHXLPLRNEAFTGXXXXXGXXETXIGWDXAIXXVAIPSTIPGNXYXXLXXXEEAXXXXXXAXEQSISXKPPYKEXXDELK

SEQ ID NO: 27 - protein, 60 aa, synthetic construct
  REGION 1..60: note = Description of Artificial Sequence: Synthetic polypeptide
  REGION 1..60: note = MISC_FEATURE - This sequence may encompass 1-15 "Gly Gly Gly Ser" repeating units
GGGSGGGSGGGSGGGSGGGSGGGSGGGSGGGSGGGSGGGSGGGSGGGSGGGSGGGSGGGS

SEQ ID NO: 28 - protein, 5 aa, synthetic construct
  REGION 1..5: note = Description of Artificial Sequence: Synthetic peptide
GGGGS

SEQ ID NO: 29 - protein, 90 aa, synthetic construct
  REGION 1..90: note = Description of Artificial Sequence: Synthetic polypeptide
  REGION 1..90: note = MISC_FEATURE - This sequence may encompass 1-15 "Gly Gly Gly Gly Gly Ser" repeating units
GGGGGSGGGGGSGGGGGSGGGGGSGGGGGSGGGGGSGGGGGSGGGGGSGGGGGSGGGGGSGGGGGSGGGGGSGGGGGSGGGGGSGGGGGS

SEQ ID NO: 30 - protein, 179 aa, synthetic construct
  REGION 1..179: note = Description of Artificial Sequence: Synthetic polypeptide
MAVLQKSMSFSLMGTLAASCLLLIALWAQEANALPVNTRCKLEVSNFQQPYIVNRTFMLAKEASLADNNTDVRLIGEKLFRGVSAKDQCYLMKQVLNFTLEDVLLPQSDRFQPYMQEVVPFLTKLSNQLSSCHISGDDQNIQKNVRRLKETVKKLGESGEIKAIGELDLLFMSLRNACV

SEQ ID NO: 31 - protein, 25 aa, synthetic construct
  REGION 1..25: note = Description of Artificial Sequence: Synthetic peptide
GGGGSGGGGSGGGGSGGGGSGGGGS

SEQ ID NO: 32 - protein, 418 aa, synthetic construct
  REGION 1..418: note = Description of Artificial Sequence: Synthetic polypeptide
MVEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGGGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 33 - protein, 438 aa, synthetic construct
  REGION 1..438: note = Description of Artificial Sequence: Synthetic polypeptide
MVEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGGGGGSGGGGSGGGGSGGGGSGGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 34 - protein, 418 aa, synthetic construct
  REGION 1..418: note = Description of Artificial Sequence: Synthetic polypeptide
MAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIGGGGSVEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKG

SEQ ID NO: 35 - protein, 538 aa, synthetic construct
  REGION 1..538: note = Description of Artificial Sequence: Synthetic polypeptide
MVEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAGGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 36 - protein, 75 aa, synthetic construct
  REGION 1..75: note = Description of Artificial Sequence: Synthetic polypeptide
  REGION 1..75: note = MISC_FEATURE - This sequence may encompass 1-15 "Gly Gly Gly Gly Ser" repeating units
GGGGSGGGGSGGGGSGGGGSGGGGSGGGGSGGGGSGGGGSGGGGSGGGGSGGGGSGGGGSGGGGSGGGGSGGGGS

SEQ ID NO: 37 - protein, 6 aa, synthetic construct
  REGION 1..6: note = Description of Artificial Sequence: Synthetic peptide
SIAKQS

SEQ ID NO: 38 - protein, 11 aa, synthetic construct
  REGION 1..11: note = Description of Artificial Sequence: Synthetic peptide
SIAKQSIAKQS

SEQ ID NO: 39 - protein, 6 aa, synthetic construct
  REGION 1..6: note = Description of Artificial Sequence: Synthetic peptide
VVKEAI

SEQ ID NO: 40 - protein, 30 aa, synthetic construct
  REGION 1..30: note = Description of Artificial Sequence: Synthetic polypeptide
  REGION 1..30: note = MISC_FEATURE - This sequence may encompass 1-15 "Gly Ser" repeating units
GSGSGSGSGSGSGSGSGSGSGSGSGSGSGS

SEQ ID NO: 41 - protein, 45 aa, synthetic construct
  REGION 1..45: note = Description of Artificial Sequence: Synthetic polypeptide
  REGION 1..45: note = MISC_FEATURE - This sequence may encompass 1-15 "Gly Gly Ser" repeating units
GGSGGSGGSGGSGGSGGSGGSGGSGGSGGSGGSGGSGGSGGSGGS